SEQUENCE LISTING (192 sequences)

SEQ ID NO: 1 - protein, 692 aa, Homo sapiens
MDVCVRLALWLLWGLLLHQGQSLSHSHSEKATGTSSGANSEESTAAEFCRIDKPLCHSEDEKLSFEAVRNIHKLMDDDANGDVDVEESDEFLREDLNYHDPTVKHSTFHGEDKLISVEDLWKAWKSSEVYNWTVDEVVQWLITYVELPQYEETFRKLQLSGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAEKQLLVAKEGAEKIKKKRNTLFGTFHVAHSSSLDDVDHKILTANSLSSVFRQALSEVTAALRERLHRWQQIEILCGFQIVNNPGIHSLVAALNIDPSWMGSTRPNPAHFIMTDDVDDMDEEIVSPLSMQSPSLQSSVRQRLTEPQHGLGSQRDLTHSDSESSLHMSDRQRVAPKPPQMSRAADEALNAMTSNGSHRLIEGVHPGSLVEKLPDSPALAKKALLALNHGLDKAHSLMELSPSAPPGGSPHLDSSRSHSPSSPDPDTPSPVGDSRALQASRNTRIPHLAGKKAVAEEDNGSIGEETDSSPGRKKFPLKIFKKPLKK

SEQ ID NO: 2 - DNA, 2079 bp, Homo sapiens
atggatgtatgcgtccgtcttgccctgtggctcctctggggactcctcctgcaccagggccagagcctcagccatagtcacagtgagaaggcgacaggaaccagctcgggggccaactctgaggagtccactgcagcagagttttgccgaattgacaagcccctgtgtcacagtgaggatgagaaactcagcttcgaggcagtccgtaacatccacaaactgatggacgatgatgccaatggtgatgtggatgtggaagaaagtgatgagttcctgagggaagacctcaattaccatgacccaacagtgaaacacagcaccttccatggtgaggataagctcatcagcgtggaggacctgtggaaggcatggaagtcatcagaagtatacaattggaccgtggatgaggtggtacagtggctgatcacatatgtggagctgcctcagtatgaggagaccttccggaagctgcagctcagtggccatgccatgccaaggctggctgtcaccaacaccaccatgacagggactgtgctgaagatgacagaccggagtcatcggcagaagctgcagctgaaggctctggatacagtgctctttgggcctcctctcttgactcgccataatcacctcaaggacttcatgctggtggtgtctatcgttattggtgtgggcggctgctggtttgcctatatccagaaccgttactccaaggagcacatgaagaagatgatgaaggacttggaggggttacaccgagctgagcagagtctgcatgaccttcaggaaaggctgcacaaggcccaggaggagcaccgcacagtggaggtggagaaggtccatctggaaaagaagctgcgcgatgagatcaaccttgctaagcaggaagcccagcggctgaaggagctgcgggagggtactgagaatgagcggagccgccaaaaatatgctgaggaggagttggagcaggttcgggaggccttgaggaaagcagagaaggagctagaatctcacagctcatggtatgctccagaggcccttcagaagtggctgcagctgacacatgaggtggaggtgcaatattacaacatcaagaagcaaaatgctgagaagcagctgctggtggccaaggagggggctgagaagataaaaaagaagagaaacacactctttggcaccttccacgtggcccacagctcttccctggatgatgtagatcataaaattctaacagctaattctctttcctctgtcttcaggcaagcactgagcgaggtgacagcagcattgcgggagcgcctgcaccgctggcaacagatcgagatcctctgtggcttccagattgtcaacaaccctggcatccactcactggtggctgccctcaacatagaccccagctggatgggcagtacacgccccaaccctgctcacttcatcatgactgacgacgtggatgacatggatgaggagattgtgtctcccttgtccatgcagtcccctagcctgcagagcagtgttcggcagcgcctgacggagccacagcatggcctgggatctcagagggatttgacccattccgattcggagtcctccctccacatgagtgaccgccagcgtgtggcccccaaacctcctcagatgagccgtgctgcagacgaggctctcaatgccatgacttccaatggcagccaccggctgatcgagggggtccacccagggtctctggtggagaaactgcctgacagccctgccctggccaagaaggcattactggcgctgaaccatgggctggacaaggcccacagcctgatggagctgagcccctcagccccacctggtggctctccacatttggattcttcccgttctcacagccccagctccccagacccagacacaccatctccagttggggacagccgagccctgcaagccagccgaaacacacgcattccccacctggctggcaagaaggctgtggctgaggaggataatggctctattggcgaggaaacagactccagcccaggccggaagaagtttcccctcaaaatctttaagaagcctcttaagaagtag

SEQ ID NO: 3 - protein, 11 aa, artificial
  aa 411-420 of STIM1ins
ANSLSSVFRQA

SEQ ID NO: 4 - DNA, 33 bp, artificial
  residues 1231-1260 of SEQ ID No 2
gctaattctctttcctctgtcttcaggcaagca

SEQ ID NO: 5 - protein, 685 aa, Homo sapiens
MDVCVRLALWLLWGLLLHQGQSLSHSHSEKATGTSSGANSEESTAAEFCRIDKPLCHSEDEKLSFEAVRNIHKLMDDDANGDVDVEESDEFLREDLNYHDPTVKHSTFHGEDKLISVEDLWKAWKSSEVYNWTVDEVVQWLITYVELPQYEETFRKLQLSGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPSLQSSVRQRLTEPQHGLGSQRDLTHSDSESSLHMSDRQRVAPKPPQMSRAADEALNAMTSNGSHRLIEGVHPGSLVEKLPDSPALAKKALLALNHGLDKAHSLMELSPSAPPGGSPHLDSSRSHSPSSPDPDTPSPVGDSRALQASRNTRIPHLAGKKAVAEEDNGSIGEETDSSPGRKKFPLKIFKKPLKK

SEQ ID NO: 6 - DNA, 2058 bp, Homo sapiens
atggatgtatgcgtccgtcttgccctgtggctcctctggggactcctcctgcaccagggccagagcctcagccatagtcacagtgagaaggcgacaggaaccagctcgggggccaactctgaggagtccactgcagcagagttttgccgaattgacaagcccctgtgtcacagtgaggatgagaaactcagcttcgaggcagtccgtaacatccacaaactgatggacgatgatgccaatggtgatgtggatgtggaagaaagtgatgagttcctgagggaagacctcaattaccatgacccaacagtgaaacacagcaccttccatggtgaggataagctcatcagcgtggaggacctgtggaaggcatggaagtcatcagaagtatacaattggaccgtggatgaggtggtacagtggctgatcacatatgtggagctgcctcagtatgaggagaccttccggaagctgcagctcagtggccatgccatgccaaggctggctgtcaccaacaccaccatgacagggactgtgctgaagatgacagaccggagtcatcggcagaagctgcagctgaaggctctggatacagtgctctttgggcctcctctcttgactcgccataatcacctcaaggacttcatgctggtggtgtctatcgttattggtgtgggcggctgctggtttgcctatatccagaaccgttactccaaggagcacatgaagaagatgatgaaggacttggaggggttacaccgagctgagcagagtctgcatgaccttcaggaaaggctgcacaaggcccaggaggagcaccgcacagtggaggtggagaaggtccatctggaaaagaagctgcgcgatgagatcaaccttgctaagcaggaagcccagcggctgaaggagctgcgggagggtactgagaatgagcggagccgccaaaaatatgctgaggaggagttggagcaggttcgggaggccttgaggaaagcagagaaggagctagaatctcacagctcatggtatgctccagaggcccttcagaagtggctgcagctgacacatgaggtggaggtgcaatattacaacatcaagaagcaaaatgctgagaagcagctgctggtggccaaggagggggctgagaagataaaaaagaagagaaacacactctttggcaccttccacgtggcccacagctcttccctggatgatgtagatcataaaattctaacagctaagcaagcactgagcgaggtgacagcagcattgcgggagcgcctgcaccgctggcaacagatcgagatcctctgtggcttccagattgtcaacaaccctggcatccactcactggtggctgccctcaacatagaccccagctggatgggcagtacacgccccaaccctgctcacttcatcatgactgacgacgtggatgacatggatgaggagattgtgtctcccttgtccatgcagtcccctagcctgcagagcagtgttcggcagcgcctgacggagccacagcatggcctgggatctcagagggatttgacccattccgattcggagtcctccctccacatgagtgaccgccagcgtgtggcccccaaacctcctcagatgagccgtgctgcagacgaggctctcaatgccatgacttccaatggcagccaccggctgatcgagggggtccacccagggtctctggtggagaaactgcctgacagccctgccctggccaagaaggcattactggcgctgaaccatgggctggacaaggcccacagcctgatggagctgagcccctcagccccacctggtggctctccacatttggattcttcccgttctcacagccccagctccccagacccagacacaccatctccagttggggacagccgagccctgcaagccagccgaaacacacgcattccccacctggctggcaagaaggctgtggctgaggaggataatggctctattggcgaggaaacagactccagcccaggccggaagaagtttcccctcaaaatctttaagaagcctcttaagaagtag

SEQ ID NO: 7 - DNA, 42 bp, artificial
  primer
ttctctttcctctgtcttcaggcaagcactgagcgaggtgac

SEQ ID NO: 8 - DNA, 33 bp, artificial
  primer
ttagctgttagaattttatgatctacatcatcc

SEQ ID NO: 9 - DNA, 20 bp, artificial
  primer
cactctttggcaccttccac

SEQ ID NO: 10 - DNA, 21 bp, artificial
  primer
gacaatctggaagccacagag

SEQ ID NO: 11 - DNA, 21 bp, artificial
  primer
ctgacacatgaggtggaggtg

SEQ ID NO: 12 - DNA, 24 bp, artificial
  primer
gcctgaagacagaggaaagagaac

SEQ ID NO: 13 - DNA, 20 bp, artificial
  primer
gtgaaacacagcaccttcca

SEQ ID NO: 14 - DNA, 21 bp, artificial
  primer
cactgtaccacctcatccacg

SEQ ID NO: 15 - DNA, 20 bp, artificial
  primer
gacgacgtggatgacatgga

SEQ ID NO: 16 - DNA, 20 bp, artificial
  primer
gatggtgtgtctgggtctgg

SEQ ID NO: 17 - DNA, 15 bp, artificial
  primer
gggcgagggcagctc

SEQ ID NO: 18 - DNA, 20 bp, artificial
  primer
aggagcagaggggtcgatag

SEQ ID NO: 19 - DNA, 20 bp, artificial
  primer
cacctctaaccaccactcgg

SEQ ID NO: 20 - DNA, 19 bp, artificial
  primer
cgggtactggtactgcgtc

SEQ ID NO: 21 - DNA, 20 bp, artificial
  primer
cgccaagcttaaagcctcca

SEQ ID NO: 22 - DNA, 21 bp, artificial
  primer
ctgatcatgagcgcaaacagg

SEQ ID NO: 23 - DNA, 20 bp, artificial
  primer
gacacctcgcagtcattcct

SEQ ID NO: 24 - DNA, 20 bp, artificial
  primer
tgatcccaccagaagcaacc

SEQ ID NO: 25 - DNA, 20 bp, artificial
  primer
gggtgctgatctccaggatg

SEQ ID NO: 26 - DNA, 20 bp, artificial
  primer
ggaggttggctcctcaatcc

SEQ ID NO: 27 - DNA, 20 bp, artificial
  primer
gagtcaacggatttggtcgt

SEQ ID NO: 28 - DNA, 20 bp, artificial
  primer
ttgattttggagggatctcg

SEQ ID NO: 29 - DNA, 13 bp, ARTIFICIAL
  Fig1A STIM1pre-mRNA first part
gtaagtaacacca

SEQ ID NO: 30 - DNA, 38 bp, artificial
  Fig1A STIM1pre-mRNA second part
caagtccatgcctgcagttctctttcctctgtcttcag

SEQ ID NO: 31 - DNA, 21 bp, artificial
  STIM1insmRNA
ttctctttcctctgtcttcag

SEQ ID NO: 32 - protein, 12 aa, artificial
  peptide for immunization
CTANSLSSFRQA

SEQ ID NO: 33 - protein, 114 aa, artificial
  1E05 VH
QVQLQQSGAELVRPGALVKLSCKASGFNIKDYYMHWVKQRPEQGLEWIGWIDPENGNTIYDPKFQGKASITADTSSNTAYLQLSSLTSEDTAVYYCAMDGFDYWGQGTTLTVSS

SEQ ID NO: 34 - protein, 112 aa, artificial
  1E05 VL
DVVMTQTPLTLSVTIGQPASISCKSSQSLLDSDGKTYLNWLLQRPGQSPKRLIYLVSKLDSGVPGRFTASGSGTDFTLRISRVETEDLGVYYCWQGTHFPLTFGTGTKLELK

SEQ ID NO: 35 - protein, 117 aa, artificial
  1F02 VH
QIQLVQSGPELKKPGETVKISCKASGYTFTNYGMYWVKQAPGKGLKWMAWINTKTGEPTYAEEFKGRFALSLETSANTAYLQINNLKNEDTASYFCANLGGGYFDVWGAGTTVTVSS

SEQ ID NO: 36 - protein, 112 aa, artificial
  1F02 VL
DVVMTQTPLSLSVTIGQTASISCKSSQSLLDSDGKTYLNWLLQRPGQSPKRLIYVVSKLDSGVPDRFTGSGSGTDFTLKISRVEAEDLGVYYCWQGTHFPLTFGAGTKLELK

SEQ ID NO: 37 - protein, 116 aa, artificial
  2F12 VH
QIQLVQSGPELKKPGETVKISCKSSVYTFTDYAMHWVKQAPGKGLKWMGWINTETGEPTYADDFKGRFAFSLETSVSTAYLHINNLKNEDTATFFCVSPTGTGDFWGQGTTLTVSS

SEQ ID NO: 38 - protein, 112 aa, artificial
  2F12 VL
DAVMTQSPLTLSVTIGQPASMSCKSSQSLLDSDGKTYLNWLLQRPGQSPKRLIYLVSKLDSGVPDRFTGSGSGTDFTLKISRVEAEDLGVYYCWQGTHFPLTFGAGTKLEIK

SEQ ID NO: 39 - protein, 112 aa, artificial
  4B05 VH
EVQLVESGGDLVKPGGSLKLSCAASGFTFNSYGMSWVRQTPDKRLEWVATITGGDGFTYYPDSVKGRFTISRDNAKNTLYLQMSSLKSEDTAIYYCTRSDYWGQGTTLTVSS

SEQ ID NO: 40 - protein, 112 aa, artificial
  4B05 VL
DVVMTQTPLTLSVTIGQPASISCKSSRSLLDTDGETYLNWLLQRPGQSPRRLIYLVSKLDSGVPDRFTGSGSGTDFTLKISRVEAEDLGVYYCWQGKHSPWTFGGGTKLEIK

SEQ ID NO: 41 - protein, 116 aa, artificial
  8E11 VH
QVQLKQSGPELVKPGASVKMSCKASGYIFSDSVITWVKQRIGQGLEWIGEIYPGSGSIYYNEKFKGKATLTADKSSNTVYIQLGSLTSEDSAVYFCANNYNGMDYWGQGTSVTVSS

SEQ ID NO: 42 - protein, 112 aa, artificial
  8E11 VL
DVVMTQTPLTLSVAIGQPASISCKSSQSLLDSSGKTYLNWLLQRPGQSPKRLIYLVSKLDSGVPDRFTGSGSGTDFTLKISRVEAEDLGIYYCVQGTHFPFTFGSGTKLEIK

SEQ ID NO: 43 - protein, 116 aa, artificial
  15G09 VH
QIQLVQSGPELKKPGETVRISCKASGYIFSNFGMNWVKQAPGKGLRWMGWINTNTGEPTYADDFKGRFVFSLEASAHTAYLQINNVKNEDMATYFCVRPTGKGDYWGRGTTLTVSS

SEQ ID NO: 44 - protein, 112 aa, artificial
  15G09 VL
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 45 - protein, 116 aa, artificial
  15H01 VH
EVQLQQSGPELVKPGASVKISCKASGYTFTDYGMDWVKQSHGKSLEWIGNINPNNGGTIYNQKFKGKATLTVDKSSSTAYMELRSLTSEDTAVYYCASPYDGGDYWGQGTSVTVSS

SEQ ID NO: 46 - protein, 112 aa, artificial
  15H01 VL
DVVMTQTPLTLSVTIGQPASISCKSSQSLLYSNGKTYLNWLLQRPGQSPKRLMYSVSKLDSGVPDRFTGSGSGTDFTLKISRVEAEDLGVYYCVQGTHFPWTFGGGTKLEIK

SEQ ID NO: 47 - protein, 116 aa, artificial
  16A08 VH
QIQLVQSGPELKKPGETVKISCKASGYTFTNYGMHWVKQAPGKALKWMGWINTKTGEPRFADDFKGRFAFSLETSANTAYLQINNLKNEDTATYFCSRAYGDYPYWGQGTTLTVSS

SEQ ID NO: 48 - protein, 112 aa, artificial
  16A08 VL
DVVMTQTPLTLSVTIGQPASISCKSSQSLLYSDGKTYLNWLLQRPGQSPKRLIYLVSKLDSGVPDRFTGSGSGTDFTLIISRVEAEDLGVYYCWQGTHFPQTFGGGTKLEIK

SEQ ID NO: 49 - protein, 5 aa, artificial
  HCDR1
DYYMH

SEQ ID NO: 50 - protein, 17 aa, artificial
  HCDR2
WIDPENGNTIYDPKFQG

SEQ ID NO: 51 - protein, 5 aa, artificial
  HCDR3
DGFDY

SEQ ID NO: 52 - protein, 7 aa, artificial
  HCDR1
GFNIKDY

SEQ ID NO: 53 - protein, 6 aa, artificial
  HCDR2
DPENGN

SEQ ID NO: 54 - protein, 5 aa, artificial
  HCDR3
DGFDY

SEQ ID NO: 55 - protein, 8 aa, artificial
  HCDR1
GFNIKDYY

SEQ ID NO: 56 - protein, 8 aa, artificial
  HCDR2
IDPENGNT

SEQ ID NO: 57 - protein, 7 aa, artificial
  HCDR3
AMDGFDY

SEQ ID NO: 58 - protein, 5 aa, artificial
  HCDR1
NYGMY

SEQ ID NO: 59 - protein, 17 aa, artificial
  HCDR2
WINTKTGEPTYAEEFKG

SEQ ID NO: 60 - protein, 8 aa, artificial
  HCDR3
LGGGYFDV

SEQ ID NO: 61 - protein, 7 aa, artificial
  HCDR1
GYTFTNY

SEQ ID NO: 62 - protein, 6 aa, artificial
  HCDR2
NTKTGE

SEQ ID NO: 63 - protein, 8 aa, artificial
  HCDR3
LGGGYFDV

SEQ ID NO: 64 - protein, 8 aa, artificial
  HCDR1
GYTFTNYG

SEQ ID NO: 65 - protein, 8 aa, artificial
  HCDR2
INTKTGEP

SEQ ID NO: 66 - protein, 10 aa, artificial
  HCDR3
ANLGGGYFDV

SEQ ID NO: 67 - protein, 5 aa, artificial
  HCDR1
DYAMH

SEQ ID NO: 68 - protein, 17 aa, artificial
  HCDR2
WINTETGEPTYADDFKG

SEQ ID NO: 69 - protein, 7 aa, artificial
  HCDR3
PTGTGDF

SEQ ID NO: 70 - protein, 7 aa, artificial
  HCDR1
VYTFTDY

SEQ ID NO: 71 - protein, 6 aa, artificial
  HCDR2
NTETGE

SEQ ID NO: 72 - protein, 7 aa, artificial
  HCDR3
PTGTGDF

SEQ ID NO: 73 - protein, 8 aa, artificial
  HCDR1
VYTFTDYA

SEQ ID NO: 74 - protein, 8 aa, artificial
  HCDR2
INTETGEP

SEQ ID NO: 75 - protein, 9 aa, artificial
  HCDR3
VSPTGTGDF

SEQ ID NO: 76 - protein, 5 aa, artificial
  HCDR1
SYGMS

SEQ ID NO: 77 - protein, 17 aa, artificial
  HCDR2
TITGGDGFTYYPDSVKG

SEQ ID NO: 78 - protein, 3 aa, artificial
  HCDR3
SDY

SEQ ID NO: 79 - protein, 7 aa, artificial
  HCDR1
GFTFNSY

SEQ ID NO: 80 - protein, 6 aa, artificial
  HCDR2
TGGDGF

SEQ ID NO: 81 - protein, 3 aa, artificial
  HCDR3
SDY

SEQ ID NO: 82 - protein, 8 aa, artificial
  HCDR1
GFTFNSYG

SEQ ID NO: 83 - protein, 8 aa, artificial
  HCDR2
ITGGDGFT

SEQ ID NO: 84 - protein, 5 aa, artificial
  HCDR3
TRSDY

SEQ ID NO: 85 - protein, 5 aa, artificial
  HCDR1
DSVIT

SEQ ID NO: 86 - protein, 17 aa, artificial
  HCDR2
EIYPGSGSIYYNEKFKG

SEQ ID NO: 87 - protein, 7 aa, artificial
  HCDR3
NYNGMDY

SEQ ID NO: 88 - protein, 7 aa, artificial
  HCDR1
GYIFSDS

SEQ ID NO: 89 - protein, 6 aa, artificial
  HCDR2
YPGSGS

SEQ ID NO: 90 - protein, 7 aa, artificial
  HCDR3
NYNGMDY

SEQ ID NO: 91 - protein, 8 aa, artificial
  HCDR1
GYIFSDSV

SEQ ID NO: 92 - protein, 8 aa, artificial
  HCDR2
IYPGSGSI

SEQ ID NO: 93 - protein, 9 aa, artificial
  HCDR3
ANNYNGMDY

SEQ ID NO: 94 - protein, 5 aa, artificial
  HCDR1
NFGMN

SEQ ID NO: 95 - protein, 17 aa, artificial
  HCDR2
WINTNTGEPTYADDFKG

SEQ ID NO: 96 - protein, 7 aa, artificial
  HCDR3
PTGKGDY

SEQ ID NO: 97 - protein, 7 aa, artificial
  HCDR1
GYIFSNF

SEQ ID NO: 98 - protein, 6 aa, artificial
  HCDR2
NTNTGE

SEQ ID NO: 99 - protein, 7 aa, artificial
  HCDR3
PTGKGDY

SEQ ID NO: 100 - protein, 8 aa, artificial
  HCDR1
GYIFSNFG

SEQ ID NO: 101 - protein, 8 aa, artificial
  HCDR2
INTNTGEP

SEQ ID NO: 102 - protein, 9 aa, artificial
  HCDR3
VRPTGKGDY

SEQ ID NO: 103 - protein, 5 aa, artificial
  HCDR1
DYGMD

SEQ ID NO: 104 - protein, 17 aa, artificial
  HCDR2
NINPNNGGTIYNQKFKG

SEQ ID NO: 105 - protein, 7 aa, artificial
  HCDR3
PYDGGDY

SEQ ID NO: 106 - protein, 7 aa, artificial
  HCDR1
GYTFTDY

SEQ ID NO: 107 - protein, 6 aa, artificial
  HCDR2
NPNNGG

SEQ ID NO: 108 - protein, 7 aa, artificial
  HCDR3
PYDGGDY

SEQ ID NO: 109 - protein, 8 aa, artificial
  HCDR1
GYTFTDYG

SEQ ID NO: 110 - protein, 8 aa, artificial
  HCDR2
INPNNGGT

SEQ ID NO: 111 - protein, 9 aa, artificial
  HCDR3
ASPYDGGDY

SEQ ID NO: 112 - protein, 5 aa, artificial
  HCDR1
NYGMH

SEQ ID NO: 113 - protein, 17 aa, artificial
  HCDR2
WINTKTGEPRFADDFKG

SEQ ID NO: 114 - protein, 7 aa, artificial
  HCDR3
AYGDYPY

SEQ ID NO: 115 - protein, 7 aa, artificial
  HCDR1
GYTFTNY

SEQ ID NO: 116 - protein, 6 aa, artificial
  HCDR2
NTKTGE

SEQ ID NO: 117 - protein, 7 aa, artificial
  HCDR3
AYGDYPY

SEQ ID NO: 118 - protein, 8 aa, artificial
  HCDR1
GYTFTNYG

SEQ ID NO: 119 - protein, 8 aa, artificial
  HCDR2
INTKTGEP

SEQ ID NO: 120 - protein, 9 aa, artificial
  HCDR3
SRAYGDYPY

SEQ ID NO: 121 - protein, 16 aa, artificial
  LCDR1
KSSQSLLDSDGKTYLN

SEQ ID NO: 122 - protein, 7 aa, artificial
  LCDR2
LVSKLDS

SEQ ID NO: 123 - protein, 9 aa, artificial
  LCDR3
WQGTHFPLT

SEQ ID NO: 124 - protein, 16 aa, artificial
  LCDR1
KSSQSLLDSDGKTYLN

SEQ ID NO: 125 - protein, 7 aa, artificial
  LCDR2
LVSKLDS

SEQ ID NO: 126 - protein, 9 aa, artificial
  LCDR3
WQGTHFPLT

SEQ ID NO: 127 - protein, 11 aa, artificial
  LCDR1
QSLLDSDGKTY

SEQ ID NO: 128 - protein, 3 aa, artificial
  LCDR2
LVS

SEQ ID NO: 129 - protein, 9 aa, artificial
  LCDR3
WQGTHFPLT

SEQ ID NO: 130 - protein, 16 aa, artificial
  LCDR1
KSSQSLLDSDGKTYLN

SEQ ID NO: 131 - protein, 7 aa, artificial
  LCDR2
VVSKLDS

SEQ ID NO: 132 - protein, 9 aa, artificial
  LCDR3
WQGTHFPLT

SEQ ID NO: 133 - protein, 16 aa, artificial
  LCDR1
KSSQSLLDSDGKTYLN

SEQ ID NO: 134 - protein, 7 aa, artificial
  LCDR2
VVSKLDS

SEQ ID NO: 135 - protein, 9 aa, artificial
  LCDR3
WQGTHFPLT

SEQ ID NO: 136 - protein, 11 aa, artificial
  LCDR1
QSLLDSDGKTY

SEQ ID NO: 137 - protein, 3 aa, artificial
  LCDR2
VVS

SEQ ID NO: 138 - protein, 9 aa, artificial
  LCDR3
WQGTHFPLT

SEQ ID NO: 139 - protein, 16 aa, artificial
  LCDR1
KSSQSLLDSDGKTYLN

SEQ ID NO: 140 - protein, 7 aa, artificial
  LCDR2
LVSKLDS

SEQ ID NO: 141 - protein, 9 aa, artificial
  LCDR3
WQGTHFPLT

SEQ ID NO: 142 - protein, 16 aa, artificial
  LCDR1
KSSQSLLDSDGKTYLN

SEQ ID NO: 143 - protein, 7 aa, artificial
  LCDR2
LVSKLDS

SEQ ID NO: 144 - protein, 9 aa, artificial
  LCDR3
WQGTHFPLT

SEQ ID NO: 145 - protein, 11 aa, artificial
  LCDR1
QSLLDSDGKTY

SEQ ID NO: 146 - protein, 3 aa, artificial
  LCDR2
LVS

SEQ ID NO: 147 - protein, 9 aa, artificial
  LCDR3
WQGTHFPLT

SEQ ID NO: 148 - protein, 16 aa, artificial
  LCDR1
KSSRSLLDTDGETYLN

SEQ ID NO: 149 - protein, 7 aa, artificial
  LCDR2
LVSKLDS

SEQ ID NO: 150 - protein, 9 aa, artificial
  LCDR3
WQGKHSPWT

SEQ ID NO: 151 - protein, 16 aa, artificial
  LCDR1
KSSRSLLDTDGETYLN

SEQ ID NO: 152 - protein, 7 aa, artificial
  LCDR2
LVSKLDS

SEQ ID NO: 153 - protein, 9 aa, artificial
  LCDR3
WQGKHSPWT

SEQ ID NO: 154 - protein, 11 aa, artificial
  LCDR1
RSLLDTDGETY

SEQ ID NO: 155 - protein, 3 aa, artificial
  LCDR2
LVS

SEQ ID NO: 156 - protein, 9 aa, artificial
  LCDR3
WQGKHSPWT

SEQ ID NO: 157 - protein, 16 aa, artificial
  LCDR1
KSSQSLLDSSGKTYLN

SEQ ID NO: 158 - protein, 7 aa, artificial
  LCDR2
LVSKLDS

SEQ ID NO: 159 - protein, 9 aa, artificial
  LCDR3
VQGTHFPFT

SEQ ID NO: 160 - protein, 16 aa, artificial
  LCDR1
KSSQSLLDSSGKTYLN

SEQ ID NO: 161 - protein, 7 aa, artificial
  LCDR2
LVSKLDS

SEQ ID NO: 162 - protein, 9 aa, artificial
  LCDR3
VQGTHFPFT

SEQ ID NO: 163 - protein, 11 aa, artificial
  LCDR1
QSLLDSSGKTY

SEQ ID NO: 164 - protein, 3 aa, artificial
  LCDR2
LVS

SEQ ID NO: 165 - protein, 9 aa, artificial
  LCDR3
VQGTHFPFT

SEQ ID NO: 166 - protein, 16 aa, artificial
  LCDR1
KSSQSLLDSDGKTYLN

SEQ ID NO: 167 - protein, 7 aa, artificial
  LCDR2
LVSKLDS

SEQ ID NO: 168 - protein, 9 aa, artificial
  LCDR3
WQGTHFPQT

SEQ ID NO: 169 - protein, 16 aa, artificial
  LCDR1
KSSQSLLDSDGKTYLN

SEQ ID NO: 170 - protein, 7 aa, artificial
  LCDR2
LVSKLDS

SEQ ID NO: 171 - protein, 9 aa, artificial
  LCDR3
WQGTHFPQT

SEQ ID NO: 172 - protein, 11 aa, artificial
  LCDR1
QSLLDSDGKTY

SEQ ID NO: 173 - protein, 3 aa, artificial
  LCDR2
LVS

SEQ ID NO: 174 - protein, 9 aa, artificial
  LCDR3
WQGTHFPQT

SEQ ID NO: 175 - protein, 16 aa, artificial
  LCDR1
KSSQSLLYSNGKTYLN

SEQ ID NO: 176 - protein, 7 aa, artificial
  LCDR2
SVSKLDS

SEQ ID NO: 177 - protein, 9 aa, artificial
  LCDR3
VQGTHFPWT

SEQ ID NO: 178 - protein, 16 aa, artificial
  LCDR1
KSSQSLLYSNGKTYLN

SEQ ID NO: 179 - protein, 7 aa, artificial
  LCDR2
SVSKLDS

SEQ ID NO: 180 - protein, 9 aa, artificial
  LCDR3
VQGTHFPWT

SEQ ID NO: 181 - protein, 11 aa, artificial
  LCDR1
QSLLYSNGKTY

SEQ ID NO: 182 - protein, 3 aa, artificial
  LCDR2
SVS

SEQ ID NO: 183 - protein, 9 aa, artificial
  LCDR3
VQGTHFPWT

SEQ ID NO: 184 - protein, 16 aa, artificial
  LCDR1
KSSQSLLYSDGKTYLN

SEQ ID NO: 185 - protein, 7 aa, artificial
  LCDR2
LVSKLDS

SEQ ID NO: 186 - protein, 9 aa, artificial
  LCDR3
WQGTHFPQT

SEQ ID NO: 187 - protein, 16 aa, artificial
  LCDR1
KSSQSLLYSDGKTYLN

SEQ ID NO: 188 - protein, 7 aa, artificial
  LCDR2
LVSKLDS

SEQ ID NO: 189 - protein, 9 aa, artificial
  LCDR3
WQGTHFPQT

SEQ ID NO: 190 - protein, 11 aa, artificial
  LCDR1
QSLLYSDGKTY

SEQ ID NO: 191 - protein, 3 aa, artificial
  LCDR2
LVS

SEQ ID NO: 192 - protein, 9 aa, artificial
  LCDR3
WQGTHFPQT

(19) United States
(12) Patent Application Publication
(10) Pub. No.: US 2024/0083990 A1
(43) Pub. Date: Mar. 14, 2024

(54) NOVEL STIM1 SPLICING VARIANTS AND USES THEREOF

(71) Applicants: UNIVERSITE BREST BRETAGNE OCCIDENTALE, BREST (FR); INSERM (INSTITUT NATIONAL DE LA SANTÉ ET DE LA RECHERCHE MÉDICALE), PARIS (FR); KALSIOM, BREST (FR)

(72) Inventors: OLIVIER MIGNEN, LO-GONNA-DAOULAS (FR); NÉLIG LE GOUX, PLOUAGAT (FR); DELPHINE BERNARD, BREST (FR); TIFFANY BERGOT, BREST (FR); MATHIEU BLERY, MÉSANGER (FR)

(21) Appl. No.: 18/274,231

(22) PCT Filed: Jan. 26, 2022
(86) PCT No.: PCT/EP2022/051783

§ 371 Date: Jul. 26, 2023

(30) Foreign Application Priority Data
Jan. 26, 2021 (EP) 21305097.4

(51) Int. Cl. C07K 16/18; A61K 39/00; A61P 35/00; C07K 14/725

(52) U.S. Cl. CPC C07K 16/18; A61K 39/4631; A61K 39/4643; A61P 35/00; C07K 14/7051; C07K 2317/21; C07K 2317/24; C07K 2317/31

(57) ABSTRACT

The present invention relates to the identification of a new splice variant of STIM1 useful as a biomarker and as a target for the treatment of diseases.

### FIELD OF THE INVENTION

The invention relates to the field of the medicine. More particularly, it relates to new biomarkers and methods of treatment, especially in the field of oncology.

### BACKGROUND OF THE INVENTION

About 95% of the coding genes in humans are subjected to alternative splicing, a highly regulated complex mechanism that can diversify the proteome by creating multiple proteins from the same gene. Deregulation of splicing is frequent in cancer, with an increase to up to 30% of alternative splicing events in tumors compared to normal samples. Splicing defects in cancer can be provoked by a deregulated expression of core splicing factors or splicing regulators, by cis-acting mutations altering specific splice junctions, or by trans-acting alterations where a somatic variant in a splicing factor leads to a global splicing defect. The latter is particularly true in myelodysplastic syndromes (MDS), a heterogeneous group of hematopoietic stem cell disorders that are characterized by an ineffective hematopoiesis due to an increased apoptosis of progenitors, and a propensity for progression in acute myeloid leukemia. Remarkably, half of MDS cases harbor a mutation in a splice factor gene, such as U2AF1, SRSF2 and SF3B1 (Splicing Factor 3B subunit 1). The latter is the most frequently mutated gene in MDS and in chronic lymphocytic leukemia (CLL). Somatic mutations of SF3B1 have been found in 70%-85% of MDS with ringed sideroblasts (MDS-RS), and in 5-18% of CLL, with an increased proportion in refractory CLL. SF3B1 mutations have also been reported in solid cancers, including uveal melanoma (14-29%), breast cancer and pancreatic cancer. While SF3B1 mutations have been associated to an overall better survival in MDS, the opposite trend has been reported in CLL, with a rapid disease progression and a shorter survival. SF3B1 is an essential component of the U2 small nuclear ribonucleoprotein particle (snRNP), which is involved in the recognition of intron-exon junctions during pre-mRNA splicing, by interacting with branch point sequences close to 3′ splice sites. Most of the cancer-associated SF3B1 mutations lead to amino acid substitutions at restricted sites in the evolutionary conserved HEAT domain, the K700E substitution being the most frequent in MDS and CLL Cancer-associated SF3B1 mutations lead to the production of hundreds of aberrant transcripts through the use of cryptic 3′ splice sites and alternative branch points, as reported by several RNA-seq analyzes performed in CLL, MDS, breast cancer and uveal melanoma. Half of these splicing variants have been predicted to be recognized and degraded by the NMD (Non-sense Mediated mRNA Decay) mechanism, while the other half would produce novel protein isoforms, some of which could be involved in the pathophysiology or/and used as biomarkers. Thus studying the functional impact caused by the significant altered transcripts produced in SF3B1-mutated cancers allows to better understand the pathophysiology of SF3B1-mutated MDS and CLL and to potentially identify new cancer antigens or therapeutic targets.

### SUMMARY OF THE INVENTION

The present invention lies in the identification of a new splice variant of STIM1, called STIM1ᵢₙₛ, that can be used as a biomarker, especially a diagnosis biomarker, a prognosis biomarker or a biomarker of response to a treatment, in the context of a disease or disorder, in particular a disease or disorder associated with a splicing defect, in particular with a mutation or alteration in a splice factor gene such as U2AF1, SRSF2, SUGP1 and SF3B1, preferably with a mutation or alteration in SF3B1 or SUGP1, more preferably a mutation in SF3B1. In a particular aspect, the disease is a cancer or a myelodysplastic syndrome. The present invention further relates to an antibody specific to this STIM1 isoform, to a peptide comprising a sequence of a fragment specific to this isoform, and to an in vitro method for detecting this isoform. In addition, the present invention provides this isoform of STIM1 as a target for treating a disease or a disorder, optionally a disease or disorder associated with a splicing defect, in particular a cancer or a myelodysplastic syndrome, for instance by a specific inhibitor (e.g., inhibiting the expression of this isoform) or a molecule targeting this isoform (e.g., an antibody specific to this isoform).

Accordingly, the present invention relates to the use of an isoform of STIM1 (Stromal interaction molecule 1) of SEQ ID NO: 1 or a fragment thereof, said fragment comprising a sequence of SEQ ID NO: 3 as a biomarker, especially a biomarker of a constitutive calcium dysregulation, preferably a biomarker of cancer or myelodysplastic syndrome.

It also relates to an antibody specific to an isoform of STIM1 of SEQ ID NO: 1, which is capable to differentially bind said isoform in comparison to an isoform of STIM1 of SEQ ID NO: 5.

In a particular aspect, the antibody comprises:
(i) a heavy chain variable domain comprising CDR 1, 2 and 3 of the heavy chain variable region of SEQ ID NO: 33 and (ii) a light chain variable domain comprising CDR 1, 2 and 3 of the light chain variable region of SEQ ID NO: 34;
(ii) a heavy chain variable domain comprising CDR 1, 2 and 3 of the heavy chain variable region of SEQ ID NO: 35 and (ii) a light chain variable domain comprising CDR 1, 2 and 3 of the light chain variable region of SEQ ID NO: 36;
(iii) a heavy chain variable domain comprising CDR 1, 2 and 3 of the heavy chain variable region of SEQ ID NO: 37 and (ii) a light chain variable domain comprising CDR 1, 2 and 3 of the light chain variable region of SEQ ID NO: 38;
(iv) a heavy chain variable domain comprising CDR 1, 2 and 3 of the heavy chain variable region of SEQ ID NO: 39 and (ii) a light chain variable domain comprising CDR 1, 2 and 3 of the light chain variable region of SEQ ID NO: 40;
(v) a heavy chain variable domain comprising CDR 1, 2 and 3 of the heavy chain variable region of SEQ ID NO: 41 and (ii) a light chain variable domain comprising CDR 1, 2 and 3 of the light chain variable region of SEQ ID NO: 42;
(vi) a heavy chain variable domain comprising CDR 1, 2 and 3 of the heavy chain variable region of SEQ ID NO: 43 and (ii) a light chain variable domain comprising CDR 1, 2 and 3 of the light chain variable region of SEQ ID NO: 44; or
(vii) a heavy chain variable domain comprising CDR 1, 2 and 3 of the heavy chain variable region of SEQ ID NO: 45 and (ii) a light chain variable domain comprising CDR 1, 2 and 3 of the light chain variable region of SEQ ID NO: 46; or
(viii) a heavy chain variable domain comprising CDR 1, 2 and 3 of the heavy chain variable region of SEQ ID NO: 47 and (ii) a light chain variable domain comprising CDR 1, 2 and 3 of the light chain variable region of SEQ ID NO: 48.

In another particular aspect, the antibody comprises:
(i) a light chain variable domain comprising a light chain CDR1 (LCDR1) comprising or consisting of an amino acid sequence of SEQ ID NO: 121; a light chain CDR2 (LCDR2) comprising or consisting of an amino acid sequence of SEQ ID NO: 122; and a light chain CDR3 (LCDR3) comprising or consisting of an amino acid sequence of SEQ ID NO: 123; and a heavy chain variable domain comprising or consisting of a heavy chain CDR1 (HCDR1) comprising or consisting of an amino acid sequence of SEQ ID NO: 49; a heavy chain CDR2 (HCDR2) comprising an amino acid sequence of SEQ ID NO: 50; and a heavy chain CDR3 (HCDR3) comprising or consisting of an amino acid sequence of SEQ ID NO: 51;
(ii) a light chain variable domain comprising a LCDR1 comprising an amino acid sequence of SEQ ID NO: 130; a LCDR2 comprising or consisting of an amino acid sequence of SEQ ID NO: 131; and a LCDR3 comprising or consisting of an amino acid sequence of SEQ ID NO: 132; and a heavy chain variable domain comprising a HCDR1 comprising or consisting of an amino acid sequence of SEQ ID NO: 58; a HCDR2 comprising or consisting of an amino acid sequence of SEQ ID NO: 59; and a HCDR3 comprising or consisting of an amino acid sequence of SEQ ID NO: 60;
(iii) a light chain variable domain comprising a LCDR1 comprising an amino acid sequence of SEQ ID NO: 139; a LCDR2 comprising or consisting of an amino acid sequence of SEQ ID NO: 140; and a LCDR3 comprising or consisting of an amino acid sequence of SEQ ID NO: 141; and a heavy chain variable domain comprising a HCDR1 comprising or consisting of an amino acid sequence of SEQ ID NO: 67; a HCDR2 comprising or consisting of an amino acid sequence of SEQ ID NO: 68; and a HCDR3 comprising or consisting of an amino acid sequence of SEQ ID NO: 69;
(iv) a light chain variable domain comprising a LCDR1 comprising an amino acid sequence of SEQ ID NO: 148; a LCDR2 comprising or consisting of an amino acid sequence of SEQ ID NO: 149; and a LCDR3 comprising or consisting of an amino acid sequence of SEQ ID NO: 150; and a heavy chain variable domain comprising a HCDR1 comprising or consisting of an amino acid sequence of SEQ ID NO: 76; a HCDR2 comprising or consisting of an amino acid sequence of SEQ ID NO: 77; and a HCDR3 comprising or consisting of an amino acid sequence of SEQ ID NO: 78;
(v) a light chain variable domain comprising a LCDR1 comprising an amino acid sequence of SEQ ID NO: 157; a LCDR2 comprising or consisting of an amino acid sequence of SEQ ID NO: 158; and a LCDR3 comprising or consisting of an amino acid sequence of SEQ ID NO: 159; and a heavy chain variable domain comprising a HCDR1 comprising or consisting of an amino acid sequence of SEQ ID NO: 85; a HCDR2 comprising or consisting of an amino acid sequence of SEQ ID NO: 86; and a HCDR3 comprising or consisting of an amino acid sequence of SEQ ID NO: 87;
(vi) a light chain variable domain comprising a LCDR1 comprising an amino acid sequence of SEQ ID NO: 166; a LCDR2 comprising or consisting of an amino acid sequence of SEQ ID NO: 167; and a LCDR3 comprising or consisting of an amino acid sequence of SEQ ID NO: 168; and a heavy chain variable domain comprising a HCDR1 comprising or consisting of an amino acid sequence of SEQ ID NO: 94; a HCDR2 comprising or consisting of an amino acid sequence of SEQ ID NO: 95; and a HCDR3 comprising or consisting of an amino acid sequence of SEQ ID NO: 96;
(vii) a light chain variable domain comprising a LCDR1 comprising an amino acid sequence of SEQ ID NO: 175; a LCDR2 comprising or consisting of an amino acid sequence of SEQ ID NO: 176; and a LCDR3 comprising or consisting of an amino acid sequence of SEQ ID NO: 177; and a heavy chain variable domain comprising a HCDR1 comprising or consisting of an amino acid sequence of SEQ ID NO: 103; a HCDR2 comprising or consisting of an amino acid sequence of SEQ ID NO: 104; and a HCDR3 comprising or consisting of an amino acid sequence of SEQ ID NO: 105; or
(viii) a light chain variable domain comprising a LCDR1 comprising an amino acid sequence of SEQ ID NO: 184; a LCDR2 comprising or consisting of an amino acid sequence of SEQ ID NO: 185; and a LCDR3 comprising or consisting of an amino acid sequence of SEQ ID NO: 186; and a heavy chain variable domain comprising a HCDR1 comprising or consisting of an amino acid sequence of SEQ ID NO: 112; a HCDR2 comprising or consisting of an amino acid sequence of SEQ ID NO: 113; and a HCDR3 comprising or consisting of an amino acid sequence of SEQ ID NO: 114.

In an additional aspect, the antibody competes with an antibody selected from the group of 1E05, 1F02, 2F12, 4B05, 8E11, 15G09, 15H01 and 16A08 for binding to a polypeptide comprising the amino acid sequence of SEQ ID NO: 1 or a fragment thereof, said fragment comprising a sequence of SEQ ID NO: 3.

Optionally, the antibody is a chimeric, humanized or human antibody.

The present invention further relates to a multispecific or bispecific antibody comprising a heavy chain variable domain and a light chain variable domain as defined any one of items (i) to (viii) herein.

The present invention also relates to a chimeric antigen receptor (CAR) comprising a heavy chain variable domain and a light chain variable domain as defined any one of items (i) to (viii) herein or a cell comprising such a CAR.

It further relates to an isolated peptide of less than 50 amino acids in length and comprising an amino acid sequence disclosed in SEQ ID NO: 3.

The present invention relates to an in vitro method for detecting an isoform of STIM1 of SEQ ID NO: 1 comprising contacting a sample with a detection mean specific to an isoform of STIM1 of SEQ ID NO: 1 and detecting the presence of the isoform of STIM1 of SEQ ID NO: 1. Preferably, the detection mean is an antibody specific to the isoform of STIM1 of SEQ ID NO: 1 as defined herein or a probe or primer specific to a coding sequence for the isoform of STIM1 of SEQ ID NO: 1, said probe or primer comprising at least 10 consecutive nucleic acids of SEQ ID NO: 4, preferably at least 7 consecutive nucleic acids of the segments in positions 1-10 of SEQ ID NO: 4.

The present invention relates to an in vitro method for detecting a cancer or a myelodysplastic syndrome or a susceptibility to develop a cancer or a myelodysplastic syndrome in a subject, wherein the method comprises detecting an isoform of STIM1 of SEQ ID NO: 1 in a sample from said subject by a method for detecting an isoform of STIM1 of SEQ ID NO: 1 as disclosed herein, the presence of the isoform of STIM1 of SEQ ID NO: 1 being indicative of a cancer or a myelodysplastic syndrome or a susceptibility to develop a cancer or a myelodysplastic syndrome in said subject.

The present invention relates to an in vitro method for providing information on a prognosis of a subject having a cancer or a myelodysplastic syndrome, wherein the method comprises detecting an isoform of STIM1 of SEQ ID NO: 1 in a sample from said subject by a method for detecting an isoform of STIM1 of SEQ ID NO: 1 as disclosed herein, the presence of the isoform of STIM1 of SEQ ID NO: 1 being indicative of the prognosis in said subject.

The present invention relates to a pharmaceutical composition comprising a modulator specific to an isoform of STIM1 of SEQ ID NO: 1 or a molecule targeting specifically an isoform of STIM1 of SEQ ID NO: 1 optionally linked to a detectable label or a drug. Optionally, the modulator can be an activator or an inhibitor. Preferably, the modulator is an inhibitor.

The present invention further relates to a modulator specific to an isoform of STIM1 of SEQ ID NO: 1 or a molecule targeting specifically an isoform of STIM1 of SEQ ID NO: 1 for use for the treatment of a disease. Optionally, the modulator can be an activator or an inhibitor. Preferably, the modulator is an inhibitor.

The present invention also relates to a vaccine composition comprising a peptide as defined herein, especially a peptide of less than 50 amino acids in length and comprising an amino acid sequence disclosed in SEQ ID NO: 3, preferably a vaccine composition for use against a cancer or a myelodysplastic syndrome.

Optionally, the modulator, e.g., activator or inhibitor, preferably inhibitor, or the molecule is an antibody specific to an isoform of STIM1 of SEQ ID NO: 1 as disclosed herein, a peptide as disclosed herein, especially a peptide of less than 50 amino acids in length and comprising an amino acid sequence disclosed in SEQ ID NO: 3, an inhibitor reducing or blocking specifically the expression of an isoform of STIM1 of SEQ ID NO: 1, a multispecific or bispecific antibody as disclosed herein, a CAR as disclosed herein or a cell comprising such a CAR. Optionally, the molecule targeting specifically an isoform of STIM1 of SEQ ID NO: 1 is an antibody or an antigen-binding fragment thereof as defined herein. Optionally, the molecule targeting specifically an isoform of STIM1 of SEQ ID NO: 1 is a multispecific or bispecific antibody. Optionally, the antibody or an antigen-binding fragment thereof as defined herein is covalently linked to a detectable label or a drug, in particular a cytotoxic drug. Optionally, the inhibitor reducing or blocking specifically the expression of an isoform of STIM1 of SEQ ID NO: 1 is selected from the group consisting of a siRNA, shRNA, antisense, ribozyme, triplex forming molecules, and aptamer specific to the mRNA encoding the isoform of STIM1 of SEQ ID NO: 1. Optionally, the molecule targeting specifically an isoform of STIM1 of SEQ ID NO: 1 is a CAR as defined herein or a cell comprising said CAR.

Optionally, the disease is associated with a splicing defect, in particular with a mutation or alteration in a splice factor gene such as U2AF1, SRSF2, SUGP1 and SF3B1, preferably with a mutation or alteration in SF3B1 or SUGP1, more preferably a mutation in SF3B1.

Preferably, the disease is a cancer or myelodysplastic syndrome. Optionally, the cancer is a hematopoietic cancer or a solid tumor, more specifically selected from the group consisting of breast cancer, lung cancer, colon cancer, bladder cancer, leukemia such as chronic lymphocytic leukemia (CLL), acute myeloid leukemia (AML), and chronic myelomonocytic leukemia (CMML), endometrium cancer, melanoma (mucosal, uveal or cutaneous), prostate cancer, pancreas cancer, glioblastoma, rectal cancer, colorectal cancer, ovary cancer, liver cancer, lung cancer, thyroid cancer, testicular cancer, myeloma (multiple myeloma), cholangiocarcinoma, nervous system cancer, uterus cancer, peritoneum cancer, digestive cancer, lymphoma and kidney cancer and the myelodysplastic syndrome is selected from the group consisting of refractory anemia, refractory anemia with ring sideroblast (RARS), refractory cytopenia with multilineage dysplasia (RCMD), refractory anemia with excess blasts, refractory anemia with excess blasts in transformation, and chronic myelomonocytic leukemia, preferably the cancer or myelodysplastic syndrome being selected from myelodysplastic syndromes, acute myeloid leukemia (AML), chronic myelomonocytic leukemia (CMML), chronic lymphocytic leukemia (CLL), refractory anemia with ring sideroblast (RARS), refractory cytopenia with multilineage dysplasia (RCMD), uveal melanoma, malignant melanoma, breast cancer, prostate cancer and bladder cancer.

In another aspect, the present invention relates to a nucleic acid encoding a heavy and/or light chain of an antibody as defined herein. Optionally, the nucleic acid encodes the heavy chain and the light chain of an antibody as defined herein.

### BRIEF DESCRIPTION OF THE FIGURES

FIG. 1. Mutations of SF3B1 in MDS and other cancers systematically induce expression of a novel STIM1 transcript, named STIM1ᵢₙₛ. A) Representation of the alternative spliced junction of STIM1 pre-mRNA and the resulting mature mRNA STIM1(canonical: STIM1-S) and STIM1ᵢₙₛ (alternative). B) Schematic representation of the STIM1ᵢₙₛ protein isoform compared to STIM2.1 isoform in which 8 amino acids are inserted in the SOAR domain of STIM1 and STIM2, respectively. C) Percent splice-in (PSI) values associated with STIM1ᵢₙₛ in different RNA-seq analysis of SF3B1^{mutated} cells. BRCA: Breast cancer, CLL: chronic lymphocytic leukemia, MDS: myelodysplastic syndromes, SKCM: Skin cancer melanoma, UVM: uveal melanoma. D) Detection of STIM1ᵢₙₛ transcript by PCR in mononuclear cells from MDS patients harbouring various SF3B1 mutations. The mutational status of SF3B1 is indicated for MDS-RS patients, as followed: mut: mutated, WT: wild type. RT-PCR was performed using primers allowing specific detection of STIMᵢₙₛ alternative transcript (F2-R2) or detection of both aberrant (upper band) and canonical (lower band) transcripts of STIM1 (F1-R1) and TMEM14C. E) Relative quantification of STIM1ᵢₙₛ transcript by quantitative RT-PCR using junction specific primers (F2-R2) in samples coming from MDS patients without RS (N=9) and MDS-RS patients carrying SF3B1 mutations (N=9) or without SF3B1 mutation (N=2). Expression level is indicated in fold-change compared to GAPDH. Error bars represent ±SEM. Statistical analysis was performed with one-way ANOVA test, ***p<0.0005, ****p<0.0001.

FIG. 2. A) Representation of STIM1 pre-mRNA and its different alternative transcripts: STIM1-S, STIM1-L, STIM1ins. B) Position of primers on STIM1 and the expect size of PCR products.

FIG. 3. Expression of SF3B1^{K700E} variant in K562 and HEK 293 cells drives the production of STIM1ᵢₙₛ splicing variant. A) Detection of STIM1ᵢₙₛ transcript in K562 stable cell lines following induction of SF3B1^{WT} or SF3B1^{K700E} expression during two or seven days. B) Detection of STIM1ᵢₙₛ transcripts in K562 cells transiently expressing SF3B1^{WT} and SF3B1^{K700E} (48 h post-transfection). RT-PCR was performed using primers allowing specific detection of STIM1ᵢₙₛ (F2-R2), alternative (upper band) and canonical (lower band) transcripts of STIM1 (F1-R1), ENOSF1 and TMEM14C. C) Quantification of STIM1ᵢₙₛ and total STIM1 transcripts by quantitative RT-PCR, using 2^{-DDCt} method (compared to GAPDH). D) Steady-state levels of STIM1 protein by Western-Blot in K562 cells. Representative Western-blot of STIM1. Blot shows bands close to 80 Kd corresponding to STIM1 for HEK cells transfected. For each condition quantification is expressed after β actine normalization. E-F-G) Same legends as B-C-D except that experiments were performed in HEK 293 cells transiently expressing SF3B1^{WT} and SF3B1^{K700E}. Error bars represent ±SEM. Statistical analysis was performed with one-way ANOVA test, ns: not significant *p<0.05, ***p<0.0005

FIG. 4. A) Only STIM1-S isoform is detected in HEK293T (left) and K562 (right) overexpressing SF3B1^{WT} or SF3B1^{K700E}. RT-PCR was performed using primer allowing amplification of both alternative transcripts STIM1-S and STIM1-L. B) STIM1ᵢₙₛ transcript is detected by RT-PCR in K562 cells transiently expressing various SF3B1 alleles (24 h post-transfection).

FIG. 5. Expression of SF3B1^{K700E} or STIM1ᵢₙₛ does not modify store-operated Ca²⁺ entry (SOCE). A) Amplitude of SOCE in cells overexpressing SF3B1^{WT} or SF3B1^{K700E} for 48 h in K562 (Left) or HEK293T (Right), which were loaded with fura-2 by measuring amplitude of fluorescence ratio variation, in comparison to cells transfected with an empty vector. B) Amplitude of SOCE in cells K562 overexpressing STIM1 and STIM1ᵢₙₛ (Left) or in HEK 293 (Right) cells using the same method. Representative recording traces are provided for each experimental conditions (A and B panels). C) Quantification of STIM1 and STIM1ᵢₙₛ expression levels in cells transiently expressing STIM1 or STIM1ins (48 h post-transfection) in K562 (Left) and HEK cells (right) by classical and quantitative RT-PCR. Error bars represent ±SEM. Statistical analysis was performed with one-way ANOVA test, ns: not significant *p<0.05, **p<0,005 ***p<0,0005, ****p>0,0001.

FIG. 6. Expression of SF3B1^{K700E} leads to an increase of the pool of STIM1 located at the plasma membrane (STIM1_{PM}) and of constitutive calcium entry (CCE). A-B) STIM1_{PM} level measured by flow cytometry analysis in non-permeabilized K562 cells (A) or HEK293T cells. B). Intact cells are labelled with a STIM1 antibody targeting the N-terminal region (STIM1 Nter 1: GOK Ab coupled to PE: A and B, left Panel) or the C-terminal region (STIM1 Cter 3: CDN3H4 clone coupled to PE: A and B, right Panel) of STIM1. Representative plots of fluorescence obtained from K562 or HEK 293 cells expressing SF3B1^{WT} or SF3B1^{K700E}. Histograms of Mean Fluorescence intensities (MFI) and mean MFI value±SEM are provided for each experimental condition. MFI values are normalized to isotype control (transfection with an empty vector) and expressed as a percentage of the average control value (% MFI). C) CCE is evaluated by the slope of the Mn²⁺ quenching of fura-2 loaded K562 cells (left) or HEK 293 cells (right) overexpressing SF3B1^{WT} or SF3B1^{K700E}. Values are expressed as percentage of the average value obtained in cells transfected with an empty vector. Representatives recording traces are presented for each experimental condition. Individual values and the mean value+/−SEM of n observations (N=6 and 4) are presented in the different histograms.

FIG. 7. Expression of STIM1ᵢₙₛ leads to an increase of STIM1_{PM} and an enhancement of constitutive calcium entry (CCE). A-B) STIM1_{PM} level measured by flow cytometry analysis in non-permeabilized K562 cells (A) or in HEK293T cells (D) transfected with an empty vector, STIM1 or STIM1ᵢₙₛ expression vector. Intact cells are labelled with two STIM1 antibodies (STIM1 Nter1 left Panel or STIM1 Cter3 right Panel). The same representation as above is used. C) CCE is evaluated by the slope of the Mn²⁺ quenching of fura-2 loaded K562 cells (Left) or HEK 293 cells (Right) overexpressing STIM1, or STIM1ᵢₙₛ. Values are expressed as percentage of the average value obtained in cells transfected with an empty vector. Individuals values and the mean value+/−SEM of n observations (N=5) are presented in the different histograms. D) CCE is evaluated by the slope of the Mn²⁺ quenching of fura-2 loaded HEK293T cells overexpressing Orai1, STIM1+Orai1 and STIM1ᵢₙₛ+Orai1 (right) or TRPC1, STIM1+TRPC1 and STIM1ᵢₙₛ+TRPC1 (left). Values are expressed as percentage of the average value obtained in cells transfected with an empty vector. Individual values and the mean value+/−SEM of n observations (N=4 or 5) are presented in the different histograms. Representatives recording traces are presented for each experimental condition in panels C and D.

FIG. 8. A) Detection of STIM1 by Western Blot of in K562 (Left) or HEK293T (right) cells transiently expressing STIM1 or STIM1ᵢₙₛ. Anti-STIM1 Nter1 antibody (GOK) or anti tag (V5 and Flag) are used for STIM1 detection. Blot shows the apparition of bands close to 75 Kd and 85 Kd corresponding respectively to the non-glycosylated and glycosylated forms of STIM1. For each condition quantification is expressed with β actine normalization. B) CCE was evaluated by slope of Mn²⁺ quenching of fura-2 loaded K562 cells overexpressing STIM BTX or STIM1ᵢₙₛ BTX splice variant.

FIG. 9. Increase of CCE is due to an enhancement in STIM1ᵢₙₛ localized at the plasma membrane as demonstrated by the block of increase of CCE by anti STIM1 antibodies. A-B) The effects of anti-STIM1 antibodies on CCE were evaluated by the slope of the Mn²⁺ quenching of fura-2 in the HEK cells overexpressing SF3B1^{WT} and SF3B1^{K700E} (A) or STIM1 and STIM1ᵢₙₛ (B). Cells were incubated for an hour with an anti-STIM1 antibody targeting the N terminal (anti-STIM1 Nter1 GOK, 10 μg/ml, left) or the C terminal (anti-STIM1 Cter3 CDN3H4, 5 μg/ml, right) domains of STIM1. For each experiment, cells were also incubated with a control isotype.

FIG. 10: Combination testing of monoclonal antibodies by flow cytometry. Unique VH/VL combinations were transiently expressed in HEK293. Supernatants were harvested and incubated with 150 000 CHO huSTIM1 CT1 or CHO huSTIM1 CT3 CHO transfectant cells per combination for 1 hour at 4° C. Cells were before incubation with Goat anti human IgG-Fc-PE secondary antibody for 30 min at 4° C. After 2 additional washes, cells were resuspended in FACS buffer containing SYTOX® Blue Dead Cell Stain. Plates were analysed on a CytoFLEX cytometer and data processed using FlowJo software. Histograms show binding of monoclonal antibodies on CHO huSTIM1 CT3 cells (black lines) or on CHO huSTIM1 CT1 cells (gray lines) compared to background signal on CHO WT cells (dotted lines). FIG. 10A: 1E05; FIG. 10B: 1F02; FIG. 10C: 2F12; FIG. 10D: 4B05; FIG. 10E: 8E11; FIG. 10F: 15G09; FIG. 10G: 15H01; and FIG. 10H: 16A08.

### DETAILED DESCRIPTION OF THE INVENTION

Deregulations of Ca²⁺ signalling appear to be a major feature of multiple diseases, including cancer and immunological diseases. Such deregulations have been shown in CLL, but have not been explored yet in MDS, except in one study that reported an increase of endoplasmic reticulum (ER) Ca²⁺ stores in low-risk MDS erythroid precursors, compared to normal controls.

The inventors thus decided to investigate Ca²⁺ homeostasis in the context of SF3B1-mutated cancers. By crossing published RNAseq data generated in SF3B1-mutated malignancies, the inventors identified an interesting novel STIM1 (Stromal Interaction Molecule 1) splice variant, called STIM1ᵢₙₛ. STIM1 is a single spanning transmembrane protein that is located mainly in the ER, and to a less extent in the plasma membrane. STIM1 plays a major role in store-operated Ca²⁺ entry (SOCE) and in regulating [Ca²⁺]i levels and homeostasis. The main molecular components of SOCE include members of STIM, ORAI and TRPC families. SOCE is regulated by the filling state of the intracellular Ca²⁺ stores such as a reduction in the intraluminal ER Ca²⁺ concentration induces the opening of calcium selective channels in the plasma membrane. There is a growing number of evidences suggesting that STIM1 is not only implicated in SOCE, but is also an important integrator of cellular signalling pathways and a regulator of store independent Ca²⁺ entry supported by ORAI and TRPC channels. Much of the understanding on the role of STIM1 in pathologies has been gained from mutations affecting critical domains of the protein, in immunodeficient patients or in some myopathies. Numerous studies have explored the implication of STIM1 expression level changes in pathological conditions such as cancer. In particular, changes in STIM1 expression were correlated to CLL severity and evolution. Of note, a second STIM1 isoform (STIM1L), which is found in human adult muscle fibres has also been described. STIM1L and STIM1 appear to have a distinct mechanism of SOCE activation and regulate differently Ca²⁺ entries. An altered splicing of STIM1, which results in a defective protein production, has been shown to lead to an absence of SOCE, as reported in a child suffering from a fatal Kaposi sarcoma. Interestingly, STIM2, a paralogue of STIM1 whose main isoform (STIM2.2; STIM2α) is an ER-resident protein mainly implicated in the regulation of basal Ca²⁺ homeostasis, display several splicing forms with opposite roles. In contrast to STIM2.2 (STIM2α), STIM2.1 (STIM2β) has been shown to prevent interaction and gating of ORAI channels, acting as a strong dominant-negative regulator of SOCE.

Since a deregulation of intracellular Ca²⁺ homeostasis has been implicated in numerous pathologies, and given that only a few studies paid attention to STIM1 alternative splicing so far, the inventors functionally characterized this new STIM1 splice variant that was specifically produced in SF3B1-mutated cells with respect to Ca²⁺ influx, in the overall context of MDS physiopathology.

The inventors showed that the isoform STIM1ᵢₙₛ is mainly present at the surface of the cells in a type II topology, i.e. with an extracellular C-terminal domain which includes the additional amino acids specific to STIM1ᵢₙₛ namely the substitution of the amino acid K in position 413 by 7 amino acids, i.e., NSLSSVFR. The amino acid sequence of this isoform is disclosed in SEQ ID NO: 1. The presence of this isoform STIM1ᵢₙₛ at the surface is specific of cells having a splicing defect found frequently in cancer and myelodysplastic syndrome. Accordingly, this isoform STIM1ᵢₙₛ can be useful as a biomarker, a pathological specific antigen and a therapeutic target. The expression of this isoform STIM1ᵢₙₛ is correlated to an increase of the constitutive entry of calcium. This increase induced by the isoform STIM1ᵢₙₛ can be blocked by an antibody directed to the C-terminal part of STIM1.

### Definition

In order that the present invention may be more readily understood, certain terms are defined hereafter. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

STIM1 is Stromal interaction molecule 1. Human STIM1 is described in UniProt under Q13586, in HGNC under 11386, STIM1 includes a signal peptide at position 1-22, a N terminal domain at positions 23-213, a transmembrane domain at positions 214-234 and a C-terminal domain at positions 235-685, as disclosed in the canonical sequence of STIM1 Uniprot Q13586-1, STIM1-S. The amino acid sequence is disclosed in NP_003147.2 and the nucleotide sequence encoding it is disclosed in NM_003156.3. A longer isoform (STIM1L) is disclosed under Uniprot G0XQ39. The amino acid sequence of STIM1L is disclosed in NP_001264890 and the nucleotide sequence encoding it is disclosed in NM_001277961.

As used herein, the term “antibody” describes a type of immunoglobulin molecule and is used in its broadest sense. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. The heavy-chain constant domains that correspond to the different classes of immunoglobulins are called alpha, delta, epsilon, gamma, and mu, respectively. Unless specifically noted otherwise, the term “antibody” includes intact immunoglobulins and “antibody fragment” or “antigen binding fragment” (such as Fab, Fab′, F(ab′)2, Fv), single chain (scFv), mutants thereof, molecules comprising an antibody portion, diabodies, linear antibodies, single chain antibodies, and any other modified configuration of the immunoglobulin molecule that comprises an antigen recognition site of the required specificity, including glycosylation variants of antibodies, amino acid sequence variants of antibodies. Preferably, the term “antibody” refers to a humanized antibody.

As used herein, an “antigen-binding fragment” of an antibody means a part of an antibody, i.e. a molecule corresponding to a portion of the structure of the antibody of the invention, that exhibits antigen-binding capacity for the antigen, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. Antigen-binding fragments of antibodies are fragments which comprise their hypervariable domains designated CDRs (Complementary Determining Regions) or part(s) thereof encompassing the recognition site for the antigen, thereby defining antigen recognition specificity.

A “Fab” fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)2 pepsin digestion product. Additional chemical couplings of antibody fragments are known to those of ordinary skill in the art. Fab and F(ab′)2 fragments lack the Fc fragment of an intact antibody, clear more rapidly from the circulation of animals, and may have less non-specific tissue binding than an intact antibody (see, e.g., Wahl et al, 1983, J. Nucl. Med. 24:316).

An “Fv” fragment is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target, although at a lower affinity than the entire binding site.

“Single-chain Fv” or “scFv” antibody binding fragments comprise the VH and VL domains of an antibody, where these domains are present in a single polypeptide chain. Generally, the Fv polypeptide further comprises a polypeptide linker between the VH and VL domains which enables the scFv to form the desired structure for target binding.

“Single domain antibodies” are composed of a single VH or VL domains which exhibit sufficient affinity to the antigen. In a specific aspect, the single domain antibody is a camelized antibody {See, e.g., Riechmann, 1999, Journal of Immunological Methods 231:25-38).

In terms of structure, an antibody may have heavy (H) chains and light (L) chains interconnected by disulfide bonds. There are two types of light chain, lambda (λ) and kappa (κ). Each heavy and light chain contains a constant region and a variable region (or “domain”). Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, and U.S. Department of Health and Human Services, 1991, which is hereby incorporated by reference). Preferably, the CDRs are defined according to Kabat method. The framework regions act to form a scaffold that provides, for positioning the CDRs in correct orientation by inter-chain, non-covalent interactions. The CDRs are primarily responsible for binding to an epitope of an antigen. The CDRs of each chain are typically referred to as “Complementarity Determining Region 1” or “CDR1”, “CDR2”, and “CDR3”, numbered sequentially starting from the N-terminus. The VL and VH domain of the antibody according to the invention may comprise four framework regions or “FR's”, which are referred to in the art and herein as “Framework region 1” or “FR1”, “FR2”, “FR3”, and “FR4”, respectively. These framework regions and complementary determining regions are preferably operably linked in the following order: FR1-CDR1-FR2-CDR2-FR3-CDR3-FR4 (from amino terminus to carboxy terminus).

An “antibody heavy chain” as used herein, refers to the larger of the two types of polypeptide chains present in antibody conformations. The CDRs of the antibody heavy chain are typically referred to as “HCDR1”, “HCDR2” and “HCDR3”. The framework regions of the antibody heavy chain are typically referred to as “HFR1”, “HFR2”, “HFR3” and “HFR4”.

An “antibody light chain,” as used herein, refers to the smaller of the two types of polypeptide chains present in antibody conformations, K and A light chains refer to the two major antibody light chain isotypes. The CDRs of the antibody light chain are typically referred to as “LCDR1”, “LCDR2” and “LCDR3”. The framework regions of the antibody light chain are typically referred to as “LFR1”, “LFR2”, “LFR3” and “LFR4”.

With regard to the binding of an antibody to a target molecule, the terms “bind” or “binding” refer to peptides, polypeptides, proteins, fusion proteins and antibodies (including antibody fragments) that recognize and contact an antigen. Preferably, it refers to an antigen-antibody type interaction. The terms “specific binding”, “specifically binds to,” “specific for,” “selectively binds” and “selective for” a particular antigen or an epitope on a particular antigen mean that the antibody recognizes and binds a specific antigen, but does not substantially recognize or bind other molecules in a sample. For example, an antibody that specifically (or preferentially) binds to STIM1_{Ins} or to a STIM1_{Ins} epitope is an antibody that binds STIM1_{Ins} for example with greater affinity, avidity, more readily, and/or with greater duration than it binds to other STIM1 splice variants or other proteins. Preferably, the term “specific binding” means the contact between an antibody and an antigen with a binding affinity equal or lower than 10⁻⁷ M. In certain aspects, antibodies bind with affinities equal or lower than 10⁻⁸ M, 10⁻⁹ M or 10⁻¹⁰ M.

When an antibody is said to “compete with” a particular monoclonal antibody, it means that the antibody competes with the monoclonal antibody in a binding assay using either recombinant STIM1_{Ins} molecules or surface expressed STIM1_{Ins} molecules. For example, if a test antibody reduces the binding of a reference antibody to a STIM1_{Ins} polypeptide or STIM1_{Ins}-expressing cell in a binding assay, the antibody is said to “compete” respectively with the reference antibody. The antibody competes with the reference antibody for binding to a polypeptide comprising the amino acid sequence of SEQ ID NO: 1 or a fragment thereof, said fragment comprising a sequence of SEQ ID NO: 3.

For the purposes herein, a “humanized” or “human” antibody refers to an antibody in which the constant and variable framework region of one or more human immunoglobulins is fused with the binding region, e.g. the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The terms “Chimeric antigen receptor” (CAR), “engineered cell receptor”, “chimeric cell receptor”, or “chimeric immune receptor” (ICR) as used herein refer to engineered receptors, which graft an antigen binding specificity onto immune cells (e.g. T cells or NK cells), thus combining the antigen binding properties of the antigen binding domain with the immunogenic activity of the immune cell, such as the lytic capacity and self-renewal of T cells. Particularly, a CAR refers to a fused protein comprising an extracellular domain able to bind an antigen, a transmembrane domain, optionally a hinge domain and at least one intracellular domain. The terms “extracellular domain able to bind an antigen”, “external domain”, “ectodomain” and “antigen binding domain” are used interchangeably herein and mean any oligopeptide or polypeptide that can bind to a targeted antigen (e.g. HLA-G isoform(s)). Particularly, the term “antigen binding domain” or “antigen-specific targeting domain” as used herein refers to the region of the CAR which targets and binds to specific antigens, for example HLA-G antigen. When a CAR is expressed in a host cell, this domain forms the extracellular domain (ectodomain) of the receptor. The antigen binding domain of a CAR typically derives from an antibody and may consist of an antigen-binding domain of a single-chain antibody (scFv) or antigen-binding fragments (Fab). The terms “intracellular domain”, “internal domain”, “cytoplasmic domain” and “intracellular signaling domain” are used interchangeably herein and mean any oligopeptide or polypeptide known to function as a domain that transmits a signal that causes activation or inhibition of a biological process in a cell. The intracellular signaling domain may generate a signal that promotes an immune effector function of the cell transduced with a nucleic acid sequence comprising a CAR, e.g. cytolytic activity and helper activity, including the secretion of cytokines. The term “transmembrane domain” means any oligopeptide or polypeptide known to span the cell membrane and that can function to link the extracellular and signaling domains. This may be a single alpha helix, a transmembrane beta barrel, a beta-helix of gramicidin A, or any other structure. Typically, the transmembrane domain denotes a single transmembrane alpha helix of a transmembrane protein, also known as an integral protein.

Within the context of this invention, “responder”, “responsive”, “positive response” or “have a therapeutic benefit” refers to a subject who responds to a treatment of cancer or myelodysplastic syndrome, for example such as the volume of the tumor is decreased, at least one of his symptoms is alleviated, or the development of the cancer or myelodysplastic syndrome is stopped, or slowed down. Typically, a subject who responds to a cancer or myelodysplastic syndrome treatment is a subject who will be completely treated (cured), i.e., a subject who will survive cancer or a patient that will survive longer. A subject who responds to a cancer or myelodysplastic syndrome treatment is also, in the sense of the present invention, a subject who has an overall survival higher than the mean overall survival known for the particular cancer, in particular in the absence of a treatment or in the presence of unsuitable treatment. It is also intended to refer to a patient who shows a good therapeutic benefit from a treatment, that is to say a longer disease-free survival, a longer overall survival, a decreased metastasis occurrence, a decreased tumor growth and/or a tumor regression in comparison to a population of patients suffering from the same cancer or myelodysplastic syndrome, in particular in the absence of a treatment.

Within the context of this invention, “non-responder”, “negative response” or “lack of response” refers to a subject who does not respond to a cancer or myelodysplastic syndrome treatment, for example such as the volume of the tumor does not substantially decrease, or the symptoms of the cancer in the subject are not alleviated, or the cancer or myelodysplastic syndrome progresses, for example the volume of the tumor increases and/or the tumor generates local or distant metastasis. The terms “non-responder” also refers to a subject who will die from cancer or myelodysplastic syndrome, or will have an overall survival lower than the mean overall survival known for the particular cancer. By “poor responder”, “negative response” or “non-responder” is intended a patient who shows a weak therapeutic benefit of the treatment, that is to say a shorter disease-free survival, a shorter overall survival, an increased metastasis occurrence and/or an increased tumor growth in comparison to a population of patients suffering from the same cancer or myelodysplastic syndrome and having the same treatment.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such a term refers to the improvement or eradication of a disease, a disorder, an infection or symptoms associated with it. In other embodiments, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect.

As used herein, the term “diagnosis” refers to the determination as to whether a subject is likely to be affected by a cancer or myelodysplastic syndrome or to the determination of whether a subject is susceptible to benefit from a treatment. The skilled artisan often makes a diagnosis on the basis of one or more diagnosis markers, the presence, absence, or amount of which is indicative of the presence or absence of the cancer. By “diagnosis”, it is also intended to refer to the provision of information useful for the diagnosis of cancer, for the prognosis of patient survival or for the determination of the response of a patient to a treatment of cancer or myelodysplastic syndrome.

As used herein, the term “marker” or “biomarker” refers to a measurable biological parameter that helps to predict the occurrence of a cancer or myelodysplastic syndrome or the efficiency of a treatment of a cancer or myelodysplastic syndrome or the prognosis of a subject having a cancer or myelodysplastic syndrome. It is in particular a measurable indicator for predicting the clinical outcome of a patient undergoing anticancer therapy or the response of a subject having cancer or myelodysplastic syndrome to a therapy.

As used herein, the terms “subject”, “individual” or “patient” are interchangeable and refer to an animal, preferably to a mammal, even more preferably to a human. However, the term “subject” can also refer to non-human animals, in particular mammals such as dogs, cats, horses, cows, pigs, sheep and non-human primates, among others.

As used herein, the term “sequence identity” or “identity” refers to an exact nucleotide to nucleotide correspondence of two polynucleotides. Percent of identity can be determined by a direct comparison of the sequence information between two molecules by aligning the sequences, counting the exact number of matches between the two aligned sequences, dividing by the length of the shorter sequence, and multiplying the result by 100.

As used herein, the term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

The term “hybridization”, as used herein, refers to “nucleic acid hybridization”. Nucleic acid hybridization depends on a principle that two single-stranded nucleic acid molecules that have complementary base sequences will form a thermodynamically favored double-stranded structure if they are mixed under the proper conditions. The double-stranded structure will be formed between two complementary single-stranded nucleic acids even if one is immobilized.

As used herein, the term “amplification” refers to the amplification of a sequence of a nucleic acid. It's a method for generating large amounts of a target sequence. In general, one or more amplification primers are annealed to a nucleic acid sequence. Using appropriate enzymes, sequences found adjacent to, or in between the primers are amplified.

The term “probe”, as used herein, means a strand of DNA or RNA of variable length (about 20-1000 bases long) which can be labelled. The probe is used in DNA or RNA samples to detect the presence of nucleotide sequences (the DNA or RNA target) that are complementary to the sequence in the probe. The term “probe”, in the context of the quantitative RT-PCR, refers to an oligonucleotide that hybridizes to a target sequence situated between the annealing sites of the two primers of the primer pair. The probe includes a detectable label, e.g., a fluorophore (Texas-Red*, Fluorescein isothiocyanate, etc.) that can be covalently attached directly to the probe oligonucleotide, e.g., located at the probe's 5′ end or at the probe's 3′ end. The probe may also include a quencher. A probe includes about 8 nucleotides, about 10 nucleotides, about 15 nucleotides, about 20 nucleotides, about 30 nucleotides, about 40 nucleotides, or about 50 nucleotides. In some embodiments, a probe includes from about 8 nucleotides to about 15 nucleotides.

The term “primer”, as used herein, means a strand of short DNA sequence that serves as a starting point for DNA synthesis. The polymerase starts polymerization at the 3′-end of the primer, creating a complementary sequence to the opposite strand. “PCR primers” are chemically synthesized oligonucleotides, with a length between 10 and 30 bases long, preferably about 20 bases long.

As used herein, the term “complementary DNA” (cDNA) refers to recombinant nucleic acid molecules synthetized by reverse transcription of a RNA molecule, for example an antisense lncRNA.

As used herein, the term “hybridizing conditions” is intended to mean those conditions of time, temperature, and pH, and the necessary amounts and concentrations of reactants and reagents, sufficient to allow at least a portion of complementary sequences to anneal with each other. As it is well known in the art, the time, temperature, and pH conditions required to accomplish hybridization depend on the size of the oligonucleotide probe or primer to be hybridized, the degree of complementarity between the oligonucleotide probe or primer and the target, the nucleotide type (e.g., RNA, or DNA) of the oligonucleotide probe or primer and the target, and the presence of other materials in the hybridization reaction mixture. The actual conditions necessary for each hybridization step are well known in the art or can be determined without undue experimentation. General parameters for specific (i.e., stringent) hybridization conditions for nucleic acids are described in Sambrook, et al., Molecular Cloning: A Laboratory Manual (3rd Edition, 2001). One of skills in the art will in particular appreciate that as the oligonucleotides become shorter, it may become necessary to adjust their length to achieve a relatively uniform melting temperature for satisfactory hybridization results.

The terms “quantity,” “amount,” and “level” are used interchangeably herein and may refer to an absolute quantification of a molecule in a sample, or to a relative quantification of a molecule in a sample, i.e., relative to another value such as relative to a reference value as taught herein, or to a range of values for the biomarker. These values or ranges can be obtained from a single patient or from a group of patients.

STIM1ᵢₙₛ as a Biomarker

STIMᵢₙₛ can be useful as biomarker. Indeed, STIM1ᵢₙₛ has been observed in the context of a splicing defect, for instance when a mutation or alteration is observed in a splice factor gene such as U2AF1, SRSF2, SUGP1 and SF3B1, preferably with a mutation or alteration in SF3B1 or SUGP1, more preferably a mutation in SF3B1. Then, the detection of the biomarker STIMᵢₙₛ can be an indirect mean to detect a splicing defect, for instance a mutation or alteration in a splice factor gene such as U2AF1, SRSF2, SUGP1 and SF3B1, preferably with a mutation or alteration in SF3B1 or SUGP1, more preferably a mutation in SF3B1. Optionally, STIMᵢₙₛ can be useful as a biomarker of constitutive calcium dysregulation.

Therefore, the present invention relates to the use of the isoform STIM1ᵢₙₛ as a biomarker. More specifically, the isoform STIM1ᵢₙₛ of SEQ ID NO: 1 or a fragment thereof comprising a sequence of SEQ ID NO: 3 can be used as biomarker. Indeed, the sequence of SEQ ID NO: 3 is specifically found in the isoform STIM1ᵢₙₛ and is not found in the other isoforms of STIM1 nor in any other protein. The fragment may comprise at least 10, 20, 30, 40, 50, 60, 70, 80, 90, or 100 amino acids of SEQ ID NO: 1 and/or less than 450, 400, 350, 300, 250, 200 or 150 amino acids of SEQ ID NO: 1.

The present invention further relates to an in vitro or ex vivo method for detecting the isoform STIM1ᵢₙₛ comprising contacting a sample with a detection mean specific to an isoform of STIM1 of SEQ ID NO:1 and detecting the presence of the isoform STIM1ᵢₙₛ.

The method may further comprise a previous step of providing a sample from a subject.

The sample can be a biological sample including fluids such as blood, plasma, serum, urine, seminal fluid samples or mixed urine and seminal fluid as well as biopsies, organs, tissues or cell samples. In one aspect, the sample comprises hematopoietic cells, especially a cell selected from the group consisting a lymphoid, myeloid, epithelial or muscular cell, for instance and non-exhaustively lymphocyte such a B lymphocyte or a T lymphocyte, a NK cell, a dendritic cell, a monocyte, a basophil, a neutrophil and an eosinophil. In another aspect, the sample comprises a tumor or cancer cell.

The isoform STIM1ᵢₙₛ can be detected at the protein level or at the nucleic acid level, especially by detecting an mRNA encoding STIM1ᵢₙₛ.

Any method known in the art can be used, for instance Northern analysis, mRNA or cDNA microarrays, polymerase chain reaction (PCR), quantitative or semi-quantitative RT-PCR, real time quantitative or semi-quantitative RT-PCR, enzyme-linked immunosorbent assay (ELISA), magnetic immunoassay (MIA), flow cytometry, microarrays, ligase chain reaction (LCR), transcription-mediated amplification (TMA), strand displacement amplification (SDA) and nucleic acid sequence based amplification (NASBA) or any such methods known in the art.

When the isoform STIM1ᵢₙₛ is detected at the protein level, the detection mean is an antibody specific to the STIM1ᵢₙₛ. Such an antibody is described herein below. Any method known in the art can be used, for instance semi-quantitative Western blots, enzyme-labeled and mediated immunoassays, such as ELISAs, biotin/avidin type assays, radioimmunoassay, immunohistochemistry, immunoelectrophoresis or immunoprecipitation, protein or antibody arrays, or flow cytometry, such as Fluorescence-activated cell sorting (FACS).

When the isoform STIM1ᵢₙₛ is detected at the nucleic acid level, especially by detecting an mRNA encoding STIM1ᵢₙₛ, the detection mean can be a probe or a primer specific to the nucleic sequence encoding the isoform STIM1ᵢₙₛ or a complementary sequence thereof. In a preferred aspect, the probe or primer comprises at least 10 consecutive nucleic acids of SEQ ID NO: 4, preferably at least 7 consecutive nucleic acids of the segments in positions 1-10 of SEQ ID NO: 4, or a complementary sequence thereof. The primer or probe can be linked to a detectable label. For instance, the primer or probe can comprise or consists of the sequence disclosed in SEQ ID NO: 12. Any method known in the art can be used, for instance hybridization (e. g., Northern blot analysis) in particular by the Nanostring method and/or by amplification (e.g., RT-PCR), in particular by quantitative or semi-quantitative RT-PCR. Other methods of Amplification include ligase chain reaction (LCR), transcription-mediated amplification (TMA), strand displacement amplification (SDA) and nucleic acid sequence-based amplification (NASBA).

In a particular aspect, the isoform STIMᵢₙₛ can be used as a biomarker of a splicing defect, for instance when a mutation or alteration is observed in a splice factor gene such as U2AF1, SRSF2, SUGP1 and SF3B1, preferably with a mutation or alteration in SF3B1 or SUGP1, more preferably a mutation in SF3B1. Accordingly, the present invention relates to a method for detecting a splicing defect, wherein the method comprises the detection of the isoform STIM1ᵢₙₛ detailed above, the detection of the isoform STIM1ᵢₙₛ being indicative of the presence of a splicing defect. On the opposite, the absence of the isoform STIM1ᵢₙₛ would be indicative of the absence of a splicing defect in the sample.

STIM1ᵢₙₛ as a Diagnostic Biomarker

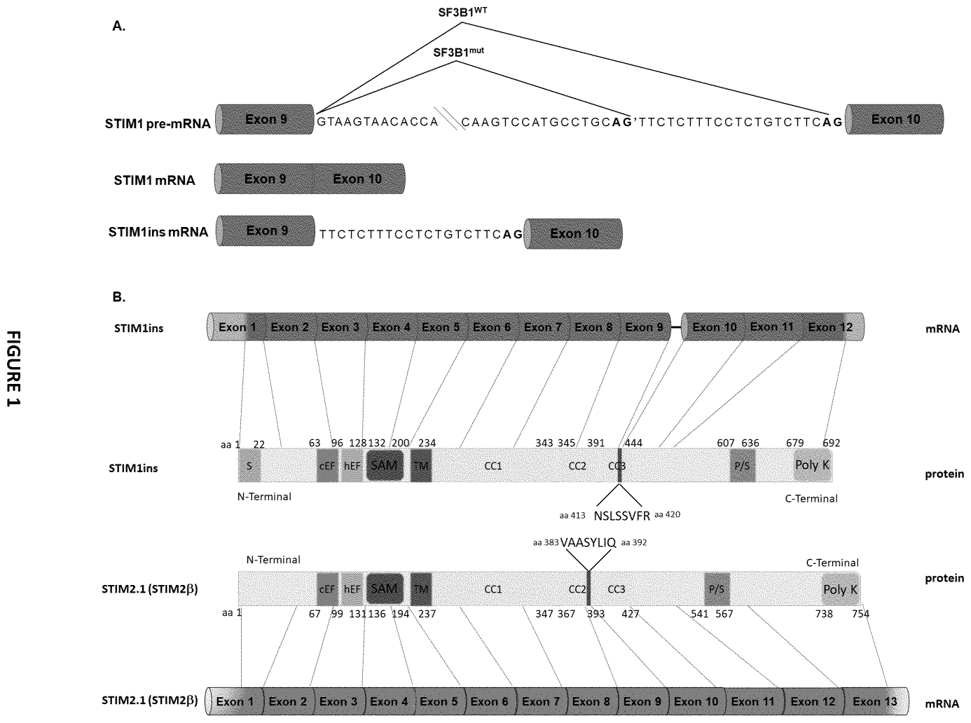

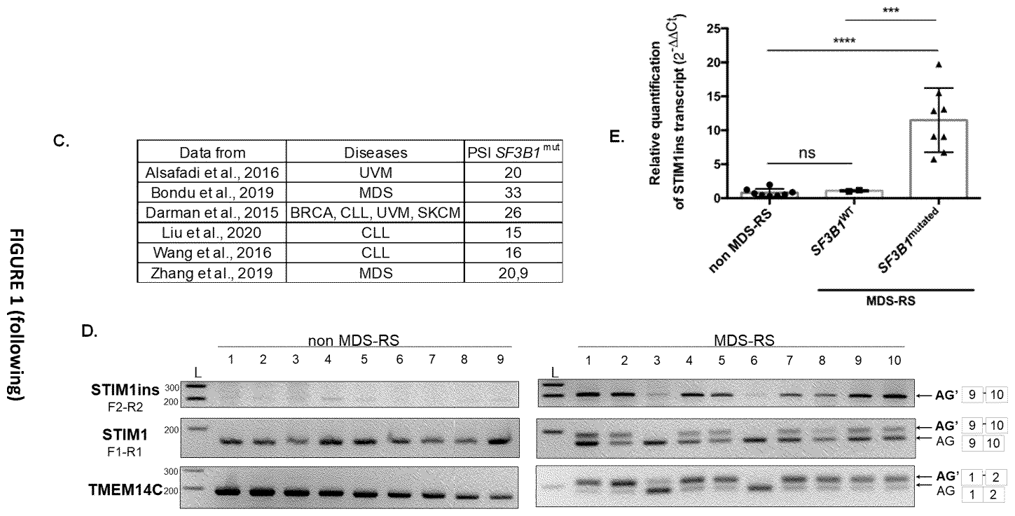

As a mutation of SF3B1 has been previously associated to be present in some diseases and disorders, then STIMᵢₙₛ can be a diagnostic biomarker indicative of a subject having or susceptible to develop these diseases and disorders. As mentioned in the Background section, half of MDS cases harbor a mutation in a splice factor gene, such as U2AF1, SRSF2 and SF3B1 and splicing defects are found quite frequently in several cancers (see FIG. 1C for illustration). The occurrence of splicing defect in these diseases has also been disclosed in the following documents
(https://www.mycancergenome.org/content/gene/sf3b1/; Obeng et al (2019, Cancer Discovery, 9, 1493-1510; Rahman et al. (2020, Cell, 180,208-208.e1;
https://cancer.sanger.ac.uk/cosmic/gene/analysis?In=SF3B1; Seiler et al. (2018, Cell Rep, 23, 282-296.e4)).

Accordingly, the isoform STIMᵢₙₛ can be used as diagnostic biomarker of a disease or disorder associated with a splicing defect, for instance a mutation or alteration in a splice factor gene such as U2AF1, SRSF2, SUGP1 and SF3B1, preferably with a mutation or alteration in SF3B1 or SUGP1, more preferably a mutation in SF3B1.

The isoform STIMᵢₙₛ can be used as diagnostic biomarker of a cancer or a myelodysplastic syndrome. In particular, isoform STIMᵢₙₛ can be used as diagnostic biomarker of a cancer or a myelodysplastic syndrome presenting a splicing defect, for instance when a mutation or alteration is observed in a splice factor gene such as U2AF1, SRSF2, SUGP1 and SF3B1, preferably with a mutation or alteration in SF3B1 or SUGP1, more preferably a mutation in SF3B1. A non-exhaustive list of cancers and myelodysplastic syndromes is provided below.

Accordingly, the present invention relates to an in vitro method for detecting a cancer or a myelodysplastic syndrome or a susceptibility to develop a cancer or a myelodysplastic syndrome in a subject, wherein the method comprises detecting the isoform STIM1ᵢₙₛ as detailed above, the detection of the isoform STIM1ᵢₙₛ being indicative of the presence of a cancer or a myelodysplastic syndrome or a susceptibility to develop a cancer or a myelodysplastic syndrome in said subject. On the opposite, the absence of the isoform STIM1ᵢₙₛ would be indicative of the absence of a cancer or a myelodysplastic syndrome or a susceptibility to develop a cancer or a myelodysplastic syndrome in said subject. The method may further comprise a previous step of providing a sample from the subject.

STIM1ᵢₙₛ as a Prognosis Biomarker

Splicing defects have been disclosed to be associated with prognosis when the disease or disorder is a myelodysplastic syndrome (Patnaik et al. (2012, Blood, 119, 5674-5677); Makishima et al. (2012, Blood, 120, 3173-3186); Mian et al (2013, haematologica, 98, 1058-1066); Papaemmanuil et al. (2011, N Engl J Med, 365, 1384-1395); Malcovati et al. (2015, Blood, 126, 233-241); Makishima et al. (2012, Blood, 119, 3203-3210)), an uveal melanoma (Martin et al. (2013), Nat Genet, 45, 933-936); Harbour et al. (2013, Nat Genet, 45, 133-135); Yavuzyigitoglu et al. (2016, Ophthalmology, 123, 1118-1128)), a chronic lymphocytic leukemia (CLL) (Baliakas et al. (2015, Leukemia, 29, 329-336); Quesada et al. (2011, Nat Genet, 44, 47-52); Fernández-Martinez et al. (2017, J Gene Med, 19(1-2)); Nadeu et al. (2016, Blood, 127, 2122-2130); Wang et al. (2016, Cancer Cell, 30, 750-763); Liu et al. (2020, Cancer Discov, 10, 806-821)), a mucosal melanoma (Queck et al. (2019, Oncotarget, 10, 930-941)) leptomeningeal melanocytic neoplasms (Küsters-Vandevelde et al. (2016, Acta Neuropathol Commun, 4, 5), a breast cancer (Liu et al. (2020, J Clin Invest, (10.1172/JCI138315); Fu et al. (2017, Oncotarget, 8, 115018-115027), a multiple myeloma (Baeur et al. (2020, (10.3324/haematol.2019.235424)), and a prolactinoma (Li et al. (2020, Nat Commun, 11, 2506)). This list is a non-exhaustive list of diseases or disorders.

Therefore, STIMᵢₙₛ can be used as a prognosis biomarker, optionally as a prognosis biomarker indicative of a positive or negative prognosis in these diseases and disorders.

Then, the present invention relates to STIMᵢₙₛ as a prognosis biomarker. It further relates to a method for assessing a prognosis in a subject or for providing information useful for assessing a prognosis in a subject comprising detecting the presence or absence of STIMᵢₙₛ in a sample from the subject, in particular as detailed above, or quantifying STIMᵢₙₛ in a sample from the subject, thereby assessing a prognosis in the subject or for providing information useful for assessing a prognosis in the subject. When the method comprises quantifying STIMᵢₙₛ in a sample from the subject, the method may further comprise the comparison of the level of expression of STIMᵢₙₛ with a level of expression of reference. Preferably, the level of expression of reference can be the level of expression of in subjects having a positive prognosis or/and the level of expression of in subjects having a negative prognosis. The level of expression can be measured by the mRNA expression or by the protein expression, in particular as detailed above for the method for detecting STIMᵢₙₛ. The method may further comprise a previous step of providing a sample from the subject.

The diseases or disorders are preferably a cancer or a myelodysplastic syndrome. Then, the subject has a cancer or a myelodysplastic syndrome. The cancer and myelodysplastic syndrome can be selected in the list provided in the present document. More specifically, the disease or disorder can be selected in the group consisting of a myelodysplastic syndrome, a melanoma, especially an uveal melanoma or a mucosal melanoma, a leukemia, especially a chronic lymphocytic leukemia (CLL), leptomeningeal melanocytic neoplasms, a solid cancer, especially a breast cancer, a multiple myeloma, and a prolactinoma.

In a particular aspect, the detection of STIMᵢₙₛ is associated with a positive prognosis when the disease or disorder is a myelodysplastic syndrome (Patnaik et al. (2012, Blood, 119, 5674-5677); Makishima et al. (2012, Blood, 120, 3173-3186); Mian et al (2013, haematologica, 98, 1058-1066); Papaemmanuil et al. (2011, N Engl J Med, 365, 1384-1395); Malcovati et al. (2015, Blood, 126, 233-241); Makishima et al. (2012, Blood, 119, 3203-3210)), an uveal melanoma (Martin et al. (2013), Nat Genet, 45, 933-936); Harbour et al. (2013, Nat Genet, 45, 133-135); Yavuzyigitoglu et al. (2016, Ophthalmology, 123, 1118-1128)).

In another particular aspect, the detection of STIMᵢₙₛ is associated with a negative prognosis when the disease or disorder is a chronic lymphocytic leukemia (CLL) (Baliakas et al. (2015, Leukemia, 29, 329-336); Quesada et al. (2011, Nat Genet, 44, 47-52); Fernández-Martinez et al. (2017, J Gene Med, 19(1-2)); Nadeu et al. (2016, Blood, 127, 2122-2130); Wang et al. (2016, Cancer Cell, 30, 750-763); Liu et al. (2020, Cancer Discov, 10, 806-821)), a mucosal melanoma (Queck et al. (2019, Oncotarget, 10, 930-941)) leptomeningeal melanocytic neoplasms (Küsters-Vandevelde et al. (2016, Acta Neuropathol Commun, 4, 5), a breast cancer (Liu et al. (2020, J Clin Invest, (10.1172/JCI138315); Fu et al. (2017, Oncotarget, 8, 115018-115027), a multiple myeloma (Baeur et al. (2020, (10.3324/haematol.2019.235424)), a prolactinoma (Li et al. (2020, Nat Commun, 11, 2506)).

STIM1ᵢₙₛ as a Biomarker of Response to a Treatment

As a mutation of SF3B1 has been previously associated to a response or an absence of response to a treatment in some diseases and disorders, then STIMᵢₙₛ can be a biomarker indicative of a response or an absence of response to a treatment in these diseases and disorders.

Therefore, the present invention relates to STIMᵢₙₛ as a biomarker of a response or lack of response to a treatment. It further relates to a method for assessing a response or lack of response to a treatment in a subject or for providing information useful for assessing a response or lack of response to a treatment in a subject comprising detecting the presence or absence of STIMᵢₙₛ in a sample from the subject, in particular as detailed above, or quantifying STIMᵢₙₛ in a sample from the subject, thereby assessing a response or lack of response to a treatment in the subject or for providing information useful for assessing a response or lack of response to a treatment in the subject. When the method comprises quantifying STIMᵢₙₛ in a sample from the subject, the method may further comprise the comparison of the level of expression of STIMᵢₙₛ with a level of expression of reference. Preferably, the level of expression of reference can be the level of expression of in subjects having a response to the treatment or/and the level of expression of in subjects having a lack of response to the treatment. The level of expression can be measured by the mRNA expression or by the protein expression, in particular as detailed above for the method for detecting STIMᵢₙₛ. The method may further comprise a previous step of providing a sample from the subject.

The diseases or disorders are preferably a cancer or a myelodysplastic syndrome. Then, the subject has a cancer or a myelodysplastic syndrome. The cancer and myelodysplastic syndrome can be selected in the list provided in the present document.

In a first aspect, the presence of STIMᵢₙₛ is indicative of a response or a likely therapeutic benefit to a treatment with luspatercept (Fenaux et al., 2020, N Engl J Med, 382, 140-151; Platzbecker et al., 2017, Lancet Oncol, 18, 1338-1347), in particular for the treatment of a myelodysplastic syndrome.

In another particular aspect, the presence of STIMᵢₙₛ is indicative of a decreased or lack of response or a likely absence of therapeutic benefit to a treatment with an immunosuppressive therapy (Zhang et al., 2020, Clin Lymphoma Myeloma Leuk, 20, 400-406.e2), in particular for the treatment of a myelodysplastic syndrome.

In another particular aspect, the presence of STIMᵢₙₛ is indicative of a decreased or lack of response or a likely absence of therapeutic benefit to a treatment with chemotherapy, especially nucleotide analogs such as fludarabine (Rossi et al. 2011, Blood, 118, 6904-6908; Wan et al., 2013, Blood, 121, 4627-4634) in the treatment of leukemia and lymphoma, in particular chronic lymphocytic leukemia, non-Hodgkin's lymphoma, acute myeloid leukemia, and acute lymphocytic leukemia, preferably chronic lymphocytic leukemia.

In another particular aspect, the presence of STIMᵢₙₛ is indicative of a decreased or lack of response or a likely absence of therapeutic benefit to a treatment with ibrutinib (Yin et al., 2019, Cancer Cell, 35, 283-296.e5) in the treatment of leukemia and lymphoma, in particular chronic lymphocytic leukemia (CLL), Waldenström's macroglobulinemia, mantle cell lymphoma, and marginal zone lymphoma, preferably chronic lymphocytic leukemia.

Antibody Specific to STIM1_{Ins}

The present invention relates to an antibody or an antigen binding fragment thereof which specifically binds to the splicing variant STIM1_{Ins} or a fragment thereof comprising the inserted sequence, preferably comprising the sequence of SEQ ID NO: 3.

By “specifically bind”, it means that the antibody is capable to differentially bind the isoform of STIM1 of SEQ ID NO: 1 in comparison to an isoform of STIM1 of SEQ ID NO: 5. For instance, the antibody has an affinity for the isoform of STIM1 of SEQ ID NO: 1 which is at least 2, 5, 10, 20, 30, 40, 50, 60, 70, 80, 90, 100, 200, 500 or 1000 fold better than its affinity for the isoform of STIM1 of SEQ ID NO: 5. The affinity can be measured by an method known in the art, and for instance by determining the KD by Surface Plasmon Resonance (SPR) technology. Optionally, the antibody binds the isoform of STIM1 of SEQ ID NO: 1 and does not bind an isoform of STIM1 of SEQ ID NO: 5. The binding can be detected by flow cytometry, in particular as detailed in example 2.

The epitope of the antibody which specifically binds to the splicing variant STIM1_{Ins} may comprise one or several of the amino acids added in the isoform STIM1ᵢₙₛ, namely amino acids from position 2 to position 9 of SEQ ID NO: 3. Optionally, the antibody may have a linear epitope comprising at least 5, 6, 7 or 8 consecutive amino acids of SEQ ID NO: 3. In a particular aspect, the linear epitope may comprise at least 5, 6, 7 or 8 consecutive amino acids of SEQ ID NO: 3 and includes amino acids from position 1 to position 5, from position 5 to position 10, from position 6 to position 10, from position 7 to position 11 of SEQ ID NO: 3.

Such an antibody can be produced by routine methods. For instance, the antibody can be produced by immunizing an animal with STIM1ᵢₙₛ or a fragment thereof, preferably a fragment comprising the sequence of SEQ ID NO: 3 and by selecting antibody that binds to STIM1ᵢₙₛ or the fragment thereof. The method may further comprise assaying the binding capacity of the selected antibody for STIM1 of SEQ ID NO: 5 and excluding the antibody capable of significantly binding STIM1 of SEQ ID NO: 5. Alternatively, the method may first discard the antibody STIM1 of SEQ ID NO: 5 before testing the capacity of the antibody to bind STIM1ᵢₙₛ or the fragment thereof.

For instance, eight antibodies which are specific STIM1ᵢₙₛ to have been prepared as specified in Example 2, namely 1E05, 1F02, 2F12, 4B05, 8E11, 15G09, 15H01 and 16A08, as defined herein.

1E05

The amino acid sequence of the heavy chain variable region of antibody 1E05 is listed as SEQ ID NO: 33 and the amino acid sequence of the light chain variable region is listed as SEQ ID NO: 34.

In a specific aspect, the invention provides an antibody that binds essentially the same epitope or determinant as monoclonal antibodies 1E05; optionally the antibody comprises the hypervariable region of antibody 1E05. Optionally, the antibody 1E05 can be characterized by the amino acid sequences and/or nucleic acid sequences encoding it. In one aspect, the monoclonal antibody comprises the Fab or F(ab′)2 portion of 1E05. Also provided is a monoclonal antibody that comprises the heavy chain variable region of 1E05. According to one aspect, the monoclonal antibody comprises the three CDRs of the heavy chain variable region of 1E05. Also provided is a monoclonal antibody that further comprises the variable light chain variable region of 1E05 or one, two or three of the CDRs of the light chain variable region of 1E05. Optionally, any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 1E05 are fused to an immunoglobulin constant region of the human IgG type, optionally a human constant region.

In another aspect, the invention provides an antibody, wherein the antibody comprises: a HCDR1 region of 1E05 comprising an amino acid sequence as set forth in Table A-1, or a sequence of at least 4, 5, 6, 7, 8, 9 or 10 contiguous amino acids thereof, optionally wherein one or more of these amino acids may be substituted by a different amino acid; a HCDR2 region of 1E05 comprising an amino acid sequence as set forth in Table A-1, or a sequence of at least 4, 5, 6, 7, 8, 9 or 10 contiguous amino acids thereof, optionally wherein one or more of these amino acids may be substituted by a different amino acid; a HCDR3 region of 1E05 comprising an amino acid sequence as set forth in Table A-1, or a sequence of at least 4, 5, 6, 7, 8, 9 or 10 contiguous amino acids thereof, optionally wherein one or more of these amino acids may be substituted by a different amino acid; a LCDR1 region of 1E05 comprising an amino acid sequence as set forth in Table A-1, or a sequence of at least 4, 5, 6, 7, 8, 9 or 10 contiguous amino acids thereof, optionally wherein one or more of these amino acids may be substituted by a different amino acid; a LCDR2 region of 1E05 comprising an amino acid sequence as set forth in Table A-1, or a sequence of at least 4, 5, 6, 7, 8, 9 or 10 contiguous amino acids thereof, optionally wherein one or more of these amino acids may be substituted by a different amino acid; a LCDR3 region of 1E05 comprising an amino acid sequence as set forth in Table A-1, or a sequence of at least 4, 5, 6, 7, 8, 9 or 10 contiguous amino acids thereof, optionally wherein one or more of these amino acids may be deleted or substituted by a different amino acid. The HCDR1, 2, 3 and LCDR1, 2, 3 sequences can optionally be specified as all (or each, independently) being those of the Kabat numbering system (as indicated in Table A-1 for each CDR), those of the Chotia numbering system as indicated in Table A-1 for each CDR), those of the IMGT numbering system as indicated in Table A-1 for each CDR), or any other suitable numbering system.

In another aspect, the invention provides an antibody that binds specifically to STIM1_{Ins}, comprising (a) the hypervariable regions of the heavy chain variable region of SEQ ID NO: 33, optionally wherein one, two, three or more amino acids are substituted by a different amino acid; and (b) the hypervariable regions of the light chain variable region of SEQ ID NO: 34, optionally wherein one, two, three or more amino acids are substituted by a different amino acid.

In another aspect, the invention provides an antibody that specifically binds STIM1_{Ins}, comprising:
(a) a heavy chain CDR 1 amino acid sequence as shown in SEQ ID NO: 49, 52 or 55, optionally wherein one, two, three or more amino acids in a CDR may be substituted by a different amino acid;
(b) a heavy chain CDR 2 amino acid sequence as shown in SEQ ID NO: 50, 53 or 56, optionally wherein one, two, three or more amino acids in a CDR may be substituted by a different amino acid;
(c) a heavy chain CDR 3 amino acid sequence as shown in SEQ ID NO: 51 or 57, optionally wherein one, two, three or more amino acids in a CDR may be substituted by a different amino acid;
(d) a light chain CDR 1 amino acid sequence as shown in SEQ ID NO: 121 or 127, optionally wherein one, two, three or more amino acids in a CDR may be substituted by a different amino acid;
(e) a light chain CDR 2 amino acid sequence as shown in SEQ ID NO: 122 or 128, optionally wherein one, two, three or more amino acids in a CDR may be substituted by a different amino acid; and/or
(f) a light chain CDR 3 amino acid sequence as shown in SEQ ID NO: 123, optionally wherein one, two, three or more amino acids in a CDR may be substituted by a different amino acid.

In a specific aspect, the antibody or the antigen binding domain comprises:
a heavy chain variable domain comprising CDR 1, 2 and 3 of the heavy chain variable region of SEQ ID NO: 33 and (ii) a light chain variable domain comprising CDR 1, 2 and 3 of the light chain variable region of SEQ ID NO: 34;

or
a light chain comprising a light chain CDR1 (LCDR1) comprising or consisting of an amino acid sequence of SEQ ID NO: 121; a light chain CDR2 (LCDR2) comprising or consisting of an amino acid sequence of SEQ ID NO: 122; and a light chain CDR3 (LCDR3) comprising or consisting of an amino acid sequence of SEQ ID NO: 123; and a heavy chain comprising or consisting of a heavy chain CDR1 (HCDR1) comprising or consisting of an amino acid sequence of SEQ ID NO: 49; a heavy chain CDR2 (HCDR2) comprising an amino acid sequence of SEQ ID NO: 50; and a heavy chain CDR3 (HCDR3) comprising or consisting of an amino acid sequence of SEQ ID NO: 51 (as defined according to Kabat).

1F02

The amino acid sequence of the heavy chain variable region of antibody 1F02 is listed as SEQ ID NO: 35 and the amino acid sequence of the light chain variable region is listed as SEQ ID NO: 36.

In a specific aspect, the invention provides an antibody that binds essentially the same epitope or determinant as monoclonal antibodies 1F02; optionally the antibody comprises the hypervariable region of antibody 1F02. Optionally, the antibody 1F02 can be characterized by the amino acid sequences and/or nucleic acid sequences encoding it. In one aspect, the monoclonal antibody comprises the Fab or F(ab′)2 portion of 1F02. Also provided is a monoclonal antibody that comprises the heavy chain variable region of 1F02. According to one aspect, the monoclonal antibody comprises the three CDRs of the heavy chain variable region of 1F02. Also provided is a monoclonal antibody that further comprises the variable light chain variable region of 1F02 or one, two or three of the CDRs of the light chain variable region of 1F02. Optionally, any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 1F02 are fused to an immunoglobulin constant region of the human IgG type, optionally a human constant region.

In another aspect, the invention provides an antibody, wherein the antibody comprises: a HCDR1 region of 1F02 comprising an amino acid sequence as set forth in Table A-2, or a sequence of at least 4, 5, 6, 7, 8, 9 or 10 contiguous amino acids thereof, optionally wherein one or more of these amino acids may be substituted by a different amino acid; a HCDR2 region of 1F02 comprising an amino acid sequence as set forth in Table A-2, or a sequence of at least 4, 5, 6, 7, 8, 9 or 10 contiguous amino acids thereof, optionally wherein one or more of these amino acids may be substituted by a different amino acid; a HCDR3 region of 1F02 comprising an amino acid sequence as set forth in Table A-2, or a sequence of at least 4, 5, 6, 7, 8, 9 or 10 contiguous amino acids thereof, optionally wherein one or more of these amino acids may be substituted by a different amino acid; a LCDR1 region of 1F02 comprising an amino acid sequence as set forth in Table A-2, or a sequence of at least 4, 5, 6, 7, 8, 9 or 10 contiguous amino acids thereof, optionally wherein one or more of these amino acids may be substituted by a different amino acid; a LCDR2 region of 1F02 comprising an amino acid sequence as set forth in Table A-2, or a sequence of at least 4, 5, 6, 7, 8, 9 or 10 contiguous amino acids thereof, optionally wherein one or more of these amino acids may be substituted by a different amino acid; a LCDR3 region of 1F02 comprising an amino acid sequence as set forth in Table A-2, or a sequence of at least 4, 5, 6, 7, 8, 9 or 10 contiguous amino acids thereof, optionally wherein one or more of these amino acids may be deleted or substituted by a different amino acid. The HCDR1, 2, 3 and LCDR1, 2, 3 sequences can optionally be specified as all (or each, independently) being those of the Kabat numbering system (as indicated in Table A-1F02 for each CDR), those of the Chotia numbering system as indicated in Table A-2 for each CDR), those of the IMGT numbering system as indicated in Table A-2 for each CDR), or any other suitable numbering system.

In another aspect, the invention provides an antibody that binds specifically to STIM1_{Ins}, comprising (a) the hypervariable regions of the heavy chain variable region of SEQ ID NO: 35, optionally wherein one, two, three or more amino acids are substituted by a different amino acid; and (b) the hypervariable regions of the light chain variable region of SEQ ID NO: 36, optionally wherein one, two, three or more amino acids are substituted by a different amino acid.

In another aspect, the invention provides an antibody that specifically binds STIM1_{Ins}, comprising:
(a) a heavy chain CDR 1 amino acid sequence as shown in SEQ ID NO: 58, 61 or 64, optionally wherein one, two, three or more amino acids in a CDR may be substituted by a different amino acid;
(b) a heavy chain CDR 2 amino acid sequence as shown in SEQ ID NO: 59, 62 or 65, optionally wherein one, two, three or more amino acids in a CDR may be substituted by a different amino acid;
(c) a heavy chain CDR 3 amino acid sequence as shown in SEQ ID NO: 60 or 66, optionally wherein one, two, three or more amino acids in a CDR may be substituted by a different amino acid;
(d) a light chain CDR 1 amino acid sequence as shown in SEQ ID NO: 130 or 136, optionally wherein one, two, three or more amino acids in a CDR may be substituted by a different amino acid;
(e) a light chain CDR 2 amino acid sequence as shown in SEQ ID NO: 131 or 137, optionally wherein one, two, three or more amino acids in a CDR may be substituted by a different amino acid; and/or
(f) a light chain CDR 3 amino acid sequence as shown in SEQ ID NO: 132, optionally wherein one, two, three or more amino acids in a CDR may be substituted by a different amino acid.

In a specific aspect, the antibody or the antigen binding domain comprises:
a heavy chain variable domain comprising CDR 1, 2 and 3 of the heavy chain variable region of SEQ ID NO: 35 and (ii) a light chain variable domain comprising CDR 1, 2 and 3 of the light chain variable region of SEQ ID NO: 36;

or
a light chain comprising a light chain CDR1 (LCDR1) comprising or consisting of an amino acid sequence of SEQ ID NO: 130; a light chain CDR2 (LCDR2) comprising or consisting of an amino acid sequence of SEQ ID NO: 131; and a light chain CDR3 (LCDR3) comprising or consisting of an amino acid sequence of SEQ ID NO: 132; and a heavy chain comprising or consisting of a heavy chain CDR1 (HCDR1) comprising or consisting of an amino acid sequence of SEQ ID NO: 58; a heavy chain CDR2 (HCDR2) comprising an amino acid sequence of SEQ ID NO: 59; and a heavy chain CDR3 (HCDR3) comprising or consisting of an amino acid sequence of SEQ ID NO: 60 (as defined according to Kabat).

2F12

The amino acid sequence of the heavy chain variable region of antibody 2F12 is listed as SEQ ID NO: 37 and the amino acid sequence of the light chain variable region is listed as SEQ ID NO: 38.

In a specific aspect, the invention provides an antibody that binds essentially the same epitope or determinant as monoclonal antibodies 2F12; optionally the antibody comprises the hypervariable region of antibody 2F12. Optionally, the antibody 2F12 can be characterized by the amino acid sequences and/or nucleic acid sequences encoding it. In one aspect, the monoclonal antibody comprises the Fab or F(ab′)2 portion of 2F12. Also provided is a monoclonal antibody that comprises the heavy chain variable region of 2F12. According to one aspect, the monoclonal antibody comprises the three CDRs of the heavy chain variable region of 2F12. Also provided is a monoclonal antibody that further comprises the variable light chain variable region of 2F12 or one, two or three of the CDRs of the light chain variable region of 2F12. Optionally, any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 2F12 are fused to an immunoglobulin constant region of the human IgG type, optionally a human constant region.

In another aspect, the invention provides an antibody, wherein the antibody comprises: a HCDR1 region of 2F12 comprising an amino acid sequence as set forth in Table A-3, or a sequence of at least 4, 5, 6, 7, 8, 9 or 10 contiguous amino acids thereof, optionally wherein one or more of these amino acids may be substituted by a different amino acid; a HCDR2 region of 2F12 comprising an amino acid sequence as set forth in Table A-3, or a sequence of at least 4, 5, 6, 7, 8, 9 or 10 contiguous amino acids thereof, optionally wherein one or more of these amino acids may be substituted by a different amino acid; a HCDR3 region of 2F12 comprising an amino acid sequence as set forth in Table A-3, or a sequence of at least 4, 5, 6, 7, 8, 9 or 10 contiguous amino acids thereof, optionally wherein one or more of these amino acids may be substituted by a different amino acid; a LCDR1 region of 2F12 comprising an amino acid sequence as set forth in Table A-3, or a sequence of at least 4, 5, 6, 7, 8, 9 or 10 contiguous amino acids thereof, optionally wherein one or more of these amino acids may be substituted by a different amino acid; a LCDR2 region of 2F12 comprising an amino acid sequence as set forth in Table A-3, or a sequence of at least 4, 5, 6, 7, 8, 9 or 10 contiguous amino acids thereof, optionally wherein one or more of these amino acids may be substituted by a different amino acid; a LCDR3 region of 2F12 comprising an amino acid sequence as set forth in Table A-3, or a sequence of at least 4, 5, 6, 7, 8, 9 or 10 contiguous amino acids thereof, optionally wherein one or more of these amino acids may be deleted or substituted by a different amino acid. The HCDR1, 2, 3 and LCDR1, 2, 3 sequences can optionally be specified as all (or each, independently) being those of the Kabat numbering system (as indicated in Table A-3 for each CDR), those of the Chotia numbering system as indicated in Table A-3 for each CDR), those of the IMGT numbering system as indicated in Table A-3 for each CDR), or any other suitable numbering system.

In another aspect, the invention provides an antibody that binds specifically to STIM1_{Ins}, comprising (a) the hypervariable regions of the heavy chain variable region of SEQ ID NO: 37, optionally wherein one, two, three or more amino acids are substituted by a different amino acid; and (b) the hypervariable regions of the light chain variable region of SEQ ID NO: 38, optionally wherein one, two, three or more amino acids are substituted by a different amino acid.

In another aspect, the invention provides an antibody that specifically binds STIM1_{Ins}, comprising:
(a) a heavy chain CDR 1 amino acid sequence as shown in SEQ ID NO: 67, 70 or 73, optionally wherein one, two, three or more amino acids in a CDR may be substituted by a different amino acid;
(b) a heavy chain CDR 2 amino acid sequence as shown in SEQ ID NO: 68, 71 or 74, optionally wherein one, two, three or more amino acids in a CDR may be substituted by a different amino acid;
(c) a heavy chain CDR 3 amino acid sequence as shown in SEQ ID NO: 69 or 75, optionally wherein one, two, three or more amino acids in a CDR may be substituted by a different amino acid;
(d) a light chain CDR 1 amino acid sequence as shown in SEQ ID NO: 139 or 145, optionally wherein one, two, three or more amino acids in a CDR may be substituted by a different amino acid;
(e) a light chain CDR 2 amino acid sequence as shown in SEQ ID NO: 140 or 146, optionally wherein one, two, three or more amino acids in a CDR may be substituted by a different amino acid; and/or
(f) a light chain CDR 3 amino acid sequence as shown in SEQ ID NO: 141, optionally wherein one, two, three or more amino acids in a CDR may be substituted by a different amino acid.

In a specific aspect, the antibody or the antigen binding domain comprises:
a heavy chain variable domain comprising CDR 1, 2 and 3 of the heavy chain variable region of SEQ ID NO: 37 and (ii) a light chain variable domain comprising CDR 1, 2 and 3 of the light chain variable region of SEQ ID NO: 38;

or
a light chain comprising a light chain CDR1 (LCDR1) comprising or consisting of an amino acid sequence of SEQ ID NO: 139; a light chain CDR2 (LCDR2) comprising or consisting of an amino acid sequence of SEQ ID NO: 140; and a light chain CDR3 (LCDR3) comprising or consisting of an amino acid sequence of SEQ ID NO: 141; and a heavy chain comprising or consisting of a heavy chain CDR1 (HCDR1) comprising or consisting of an amino acid sequence of SEQ ID NO: 67; a heavy chain CDR2 (HCDR2) comprising an amino acid sequence of SEQ ID NO: 68; and a heavy chain CDR3 (HCDR3) comprising or consisting of an amino acid sequence of SEQ ID NO: 69 (as defined according to Kabat).

4B05

The amino acid sequence of the heavy chain variable region of antibody 4B05 is listed as SEQ ID NO: 39 and the amino acid sequence of the light chain variable region is listed as SEQ ID NO: 40.

In a specific aspect, the invention provides an antibody that binds essentially the same epitope or determinant as monoclonal antibodies 4B05; optionally the antibody comprises the hypervariable region of antibody 4B05. Optionally, the antibody 4B05 can be characterized by the amino acid sequences and/or nucleic acid sequences encoding it. In one aspect, the monoclonal antibody comprises the Fab or F(ab′)2 portion of 4B05. Also provided is a monoclonal antibody that comprises the heavy chain variable region of 4B05. According to one aspect, the monoclonal antibody comprises the three CDRs of the heavy chain variable region of 4B05. Also provided is a monoclonal antibody that further comprises the variable light chain variable region of 4B05 or one, two or three of the CDRs of the light chain variable region of 4B05. Optionally, any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 4B05 are fused to an immunoglobulin constant region of the human IgG type, optionally a human constant region.

In another aspect, the invention provides an antibody, wherein the antibody comprises: a HCDR1 region of 4B05 comprising an amino acid sequence as set forth in Table A-4, or a sequence of at least 4, 5, 6, 7, 8, 9 or 10 contiguous amino acids thereof, optionally wherein one or more of these amino acids may be substituted by a different amino acid; a HCDR2 region of 4B05 comprising an amino acid sequence as set forth in Table A-4, or a sequence of at least 4, 5, 6, 7, 8, 9 or 10 contiguous amino acids thereof, optionally wherein one or more of these amino acids may be substituted by a different amino acid; a HCDR3 region of 4B05 comprising an amino acid sequence as set forth in Table A-4, or a sequence of at least 4, 5, 6, 7, 8, 9 or 10 contiguous amino acids thereof, optionally wherein one or more of these amino acids may be substituted by a different amino acid; a LCDR1 region of 4B05 comprising an amino acid sequence as set forth in Table A-4, or a sequence of at least 4, 5, 6, 7, 8, 9 or 10 contiguous amino acids thereof, optionally wherein one or more of these amino acids may be substituted by a different amino acid; a LCDR2 region of 4B05 comprising an amino acid sequence as set forth in Table A-4, or a sequence of at least 4, 5, 6, 7, 8, 9 or 10 contiguous amino acids thereof, optionally wherein one or more of these amino acids may be substituted by a different amino acid; a LCDR3 region of 4B05 comprising an amino acid sequence as set forth in Table A-4, or a sequence of at least 4, 5, 6, 7, 8, 9 or 10 contiguous amino acids thereof, optionally wherein one or more of these amino acids may be deleted or substituted by a different amino acid. The HCDR1, 2, 3 and LCDR1, 2, 3 sequences can optionally be specified as all (or each, independently) being those of the Kabat numbering system (as indicated in Table A-4 for each CDR), those of the Chotia numbering system as indicated in Table A-4 for each CDR), those of the IMGT numbering system as indicated in Table A-4 for each CDR), or any other suitable numbering system.

In another aspect, the invention provides an antibody that binds specifically to STIM1_{Ins}, comprising (a) the hypervariable regions of the heavy chain variable region of SEQ ID NO: 39, optionally wherein one, two, three or more amino acids are substituted by a different amino acid; and (b) the hypervariable regions of the light chain variable region of SEQ ID NO: 40, optionally wherein one, two, three or more amino acids are substituted by a different amino acid.

In another aspect, the invention provides an antibody that specifically binds STIM1_{Ins}, comprising:
(a) a heavy chain CDR 1 amino acid sequence as shown in SEQ ID NO: 76, 79 or 82, optionally wherein one, two, three or more amino acids in a CDR may be substituted by a different amino acid;
(b) a heavy chain CDR 2 amino acid sequence as shown in SEQ ID NO: 77, 80 or 83, optionally wherein one, two, three or more amino acids in a CDR may be substituted by a different amino acid;
(c) a heavy chain CDR 3 amino acid sequence as shown in SEQ ID NO: 78 or 84, optionally wherein one, two, three or more amino acids in a CDR may be substituted by a different amino acid;
(d) a light chain CDR 1 amino acid sequence as shown in SEQ ID NO: 148 or 154, optionally wherein one, two, three or more amino acids in a CDR may be substituted by a different amino acid;
(e) a light chain CDR 2 amino acid sequence as shown in SEQ ID NO: 149 or 155, optionally wherein one, two, three or more amino acids in a CDR may be substituted by a different amino acid; and/or
(f) a light chain CDR 3 amino acid sequence as shown in SEQ ID NO: 150, optionally wherein one, two, three or more amino acids in a CDR may be substituted by a different amino acid.

In a specific aspect, the antibody or the antigen binding domain comprises:
a heavy chain variable domain comprising CDR 1, 2 and 3 of the heavy chain variable region of SEQ ID NO: 39 and (ii) a light chain variable domain comprising CDR 1, 2 and 3 of the light chain variable region of SEQ ID NO: 40;

or
a light chain comprising a light chain CDR1 (LCDR1) comprising or consisting of an amino acid sequence of SEQ ID NO: 148; a light chain CDR2 (LCDR2) comprising or consisting of an amino acid sequence of SEQ ID NO: 149; and a light chain CDR3 (LCDR3) comprising or consisting of an amino acid sequence of SEQ ID NO: 150; and a heavy chain comprising or consisting of a heavy chain CDR1 (HCDR1) comprising or consisting of an amino acid sequence of SEQ ID NO: 76; a heavy chain CDR2 (HCDR2) comprising an amino acid sequence of SEQ ID NO: 77; and a heavy chain CDR3 (HCDR3) comprising or consisting of an amino acid sequence of SEQ ID NO: 78 (as defined according to Kabat).

8E11

The amino acid sequence of the heavy chain variable region of antibody 8E11 is listed as SEQ ID NO: 41 and the amino acid sequence of the light chain variable region is listed as SEQ ID NO: 42.

In a specific aspect, the invention provides an antibody that binds essentially the same epitope or determinant as monoclonal antibodies 8E11; optionally the antibody comprises the hypervariable region of antibody 8E11. Optionally, the antibody 8E11 can be characterized by the amino acid sequences and/or nucleic acid sequences encoding it. In one aspect, the monoclonal antibody comprises the Fab or F(ab′)2 portion of 8E11. Also provided is a monoclonal antibody that comprises the heavy chain variable region of 8E11. According to one aspect, the monoclonal antibody comprises the three CDRs of the heavy chain variable region of 8E11. Also provided is a monoclonal antibody that further comprises the variable light chain variable region of 8E11 or one, two or three of the CDRs of the light chain variable region of 8E11. Optionally, any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 8E11 are fused to an immunoglobulin constant region of the human IgG type, optionally a human constant region.

In another aspect, the invention provides an antibody, wherein the antibody comprises: a HCDR1 region of 8E11 comprising an amino acid sequence as set forth in Table A-5, or a sequence of at least 4, 5, 6, 7, 8, 9 or 10 contiguous amino acids thereof, optionally wherein one or more of these amino acids may be substituted by a different amino acid; a HCDR2 region of 8E11 comprising an amino acid sequence as set forth in Table A-5, or a sequence of at least 4, 5, 6, 7, 8, 9 or 10 contiguous amino acids thereof, optionally wherein one or more of these amino acids may be substituted by a different amino acid; a HCDR3 region of 8E11 comprising an amino acid sequence as set forth in Table A-5, or a sequence of at least 4, 5, 6, 7, 8, 9 or 10 contiguous amino acids thereof, optionally wherein one or more of these amino acids may be substituted by a different amino acid; a LCDR1 region of 8E11 comprising an amino acid sequence as set forth in Table A-5, or a sequence of at least 4, 5, 6, 7, 8, 9 or 10 contiguous amino acids thereof, optionally wherein one or more of these amino acids may be substituted by a different amino acid; a LCDR2 region of 8E11 comprising an amino acid sequence as set forth in Table A-5, or a sequence of at least 4, 5, 6, 7, 8, 9 or 10 contiguous amino acids thereof, optionally wherein one or more of these amino acids may be substituted by a different amino acid; a LCDR3 region of 8E11 comprising an amino acid sequence as set forth in Table A-5, or a sequence of at least 4, 5, 6, 7, 8, 9 or 10 contiguous amino acids thereof, optionally wherein one or more of these amino acids may be deleted or substituted by a different amino acid. The HCDR1, 2, 3 and LCDR1, 2, 3 sequences can optionally be specified as all (or each, independently) being those of the Kabat numbering system (as indicated in Table A-5 for each CDR), those of the Chotia numbering system as indicated in Table A-5 for each CDR), those of the IMGT numbering system as indicated in Table A-5 for each CDR), or any other suitable numbering system.

In another aspect, the invention provides an antibody that binds specifically to STIM1_{Ins}, comprising (a) the hypervariable regions of the heavy chain variable region of SEQ ID NO: 41, optionally wherein one, two, three or more amino acids are substituted by a different amino acid; and (b) the hypervariable regions of the light chain variable region of SEQ ID NO: 42, optionally wherein one, two, three or more amino acids are substituted by a different amino acid.

In another aspect, the invention provides an antibody that specifically binds STIM1_{Ins}, comprising:
(a) a heavy chain CDR 1 amino acid sequence as shown in SEQ ID NO: 85, 88 or 91, optionally wherein one, two, three or more amino acids in a CDR may be substituted by a different amino acid;
(b) a heavy chain CDR 2 amino acid sequence as shown in SEQ ID NO: 86, 89 or 92, optionally wherein one, two, three or more amino acids in a CDR may be substituted by a different amino acid;
(c) a heavy chain CDR 3 amino acid sequence as shown in SEQ ID NO: 87 or 93, optionally wherein one, two, three or more amino acids in a CDR may be substituted by a different amino acid;
(d) a light chain CDR 1 amino acid sequence as shown in SEQ ID NO: 157 or 163, optionally wherein one, two, three or more amino acids in a CDR may be substituted by a different amino acid;
(e) a light chain CDR 2 amino acid sequence as shown in SEQ ID NO: 158 or 164, optionally wherein one, two, three or more amino acids in a CDR may be substituted by a different amino acid; and/or
(f) a light chain CDR 3 amino acid sequence as shown in SEQ ID NO: 159, optionally wherein one, two, three or more amino acids in a CDR may be substituted by a different amino acid.

In a specific aspect, the antibody or the antigen binding domain comprises:
a heavy chain variable domain comprising CDR 1, 2 and 3 of the heavy chain variable region of SEQ ID NO: 41 and (ii) a light chain variable domain comprising CDR 1, 2 and 3 of the light chain variable region of SEQ ID NO: 42;

or
a light chain comprising a light chain CDR1 (LCDR1) comprising or consisting of an amino acid sequence of SEQ ID NO: 157; a light chain CDR2 (LCDR2) comprising or consisting of an amino acid sequence of SEQ ID NO: 158; and a light chain CDR3 (LCDR3) comprising or consisting of an amino acid sequence of SEQ ID NO: 159; and a heavy chain comprising or consisting of a heavy chain CDR1 (HCDR1) comprising or consisting of an amino acid sequence of SEQ ID NO: 85; a heavy chain CDR2 (HCDR2) comprising an amino acid sequence of SEQ ID NO: 86; and a heavy chain CDR3 (HCDR3) comprising or consisting of an amino acid sequence of SEQ ID NO: 87 (as defined according to Kabat).

15G09

The amino acid sequence of the heavy chain variable region of antibody 15G09 is listed as SEQ ID NO: 43 and the amino acid sequence of the light chain variable region is listed as SEQ ID NO: 44.

In a specific aspect, the invention provides an antibody that binds essentially the same epitope or determinant as monoclonal antibodies 15G09; optionally the antibody comprises the hypervariable region of antibody 15G09. Optionally, the antibody 15G09 can be characterized by the amino acid sequences and/or nucleic acid sequences encoding it. In one aspect, the monoclonal antibody comprises the Fab or F(ab′)2 portion of 15G09. Also provided is a monoclonal antibody that comprises the heavy chain variable region of 15G09. According to one aspect, the monoclonal antibody comprises the three CDRs of the heavy chain variable region of 15G09. Also provided is a monoclonal antibody that further comprises the variable light chain variable region of 15G09 or one, two or three of the CDRs of the light chain variable region of 15G09. Optionally, any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 15G09 are fused to an immunoglobulin constant region of the human IgG type, optionally a human constant region.

In another aspect, the invention provides an antibody, wherein the antibody comprises: a HCDR1 region of 15G09 comprising an amino acid sequence as set forth in Table A-6, or a sequence of at least 4, 5, 6, 7, 8, 9 or 10 contiguous amino acids thereof, optionally wherein one or more of these amino acids may be substituted by a different amino acid; a HCDR2 region of 15G09 comprising an amino acid sequence as set forth in Table A-6, or a sequence of at least 4, 5, 6, 7, 8, 9 or 10 contiguous amino acids thereof, optionally wherein one or more of these amino acids may be substituted by a different amino acid; a HCDR3 region of 15G09 comprising an amino acid sequence as set forth in Table A-6, or a sequence of at least 4, 5, 6, 7, 8, 9 or 10 contiguous amino acids thereof, optionally wherein one or more of these amino acids may be substituted by a different amino acid; a LCDR1 region of 15G09 comprising an amino acid sequence as set forth in Table A-6, or a sequence of at least 4, 5, 6, 7, 8, 9 or 10 contiguous amino acids thereof, optionally wherein one or more of these amino acids may be substituted by a different amino acid; a LCDR2 region of 15G09 comprising an amino acid sequence as set forth in Table A-6, or a sequence of at least 4, 5, 6, 7, 8, 9 or 10 contiguous amino acids thereof, optionally wherein one or more of these amino acids may be substituted by a different amino acid; a LCDR3 region of 15G09 comprising an amino acid sequence as set forth in Table A-6, or a sequence of at least 4, 5, 6, 7, 8, 9 or 10 contiguous amino acids thereof, optionally wherein one or more of these amino acids may be deleted or substituted by a different amino acid. The HCDR1, 2, 3 and LCDR1, 2, 3 sequences can optionally be specified as all (or each, independently) being those of the Kabat numbering system (as indicated in Table A-6 for each CDR), those of the Chotia numbering system as indicated in Table A-6 for each CDR), those of the IMGT numbering system as indicated in Table A-6 for each CDR), or any other suitable numbering system.

In another aspect, the invention provides an antibody that binds specifically to STIM1_{Ins}, comprising (a) the hypervariable regions of the heavy chain variable region of SEQ ID NO: 43, optionally wherein one, two, three or more amino acids are substituted by a different amino acid; and (b) the hypervariable regions of the light chain variable region of SEQ ID NO: 44, optionally wherein one, two, three or more amino acids are substituted by a different amino acid.

In another aspect, the invention provides an antibody that specifically binds STIM1_{Ins}, comprising:
(a) a heavy chain CDR 1 amino acid sequence as shown in SEQ ID NO: 94, 97 or 100, optionally wherein one, two, three or more amino acids in a CDR may be substituted by a different amino acid;
(b) a heavy chain CDR 2 amino acid sequence as shown in SEQ ID NO: 95, 98 or 101, optionally wherein one, two, three or more amino acids in a CDR may be substituted by a different amino acid;
(c) a heavy chain CDR 3 amino acid sequence as shown in SEQ ID NO: 96 or 102, optionally wherein one, two, three or more amino acids in a CDR may be substituted by a different amino acid;
(d) a light chain CDR 1 amino acid sequence as shown in SEQ ID NO: 166 or 172, optionally wherein one, two, three or more amino acids in a CDR may be substituted by a different amino acid;
(e) a light chain CDR 2 amino acid sequence as shown in SEQ ID NO: 167 or 173, optionally wherein one, two, three or more amino acids in a CDR may be substituted by a different amino acid; and/or
(f) a light chain CDR 3 amino acid sequence as shown in SEQ ID NO: 168, optionally wherein one, two, three or more amino acids in a CDR may be substituted by a different amino acid.

In a specific aspect, the antibody or the antigen binding domain comprises:
a heavy chain variable domain comprising CDR 1, 2 and 3 of the heavy chain variable region of SEQ ID NO: 43 and (ii) a light chain variable domain comprising CDR 1, 2 and 3 of the light chain variable region of SEQ ID NO: 44;

or
a light chain comprising a light chain CDR1 (LCDR1) comprising or consisting of an amino acid sequence of SEQ ID NO: 166; a light chain CDR2 (LCDR2) comprising or consisting of an amino acid sequence of SEQ ID NO: 167; and a light chain CDR3 (LCDR3) comprising or consisting of an amino acid sequence of SEQ ID NO: 168; and a heavy chain comprising or consisting of a heavy chain CDR1 (HCDR1) comprising or consisting of an amino acid sequence of SEQ ID NO: 94; a heavy chain CDR2 (HCDR2) comprising an amino acid sequence of SEQ ID NO: 95; and a heavy chain CDR3 (HCDR3) comprising or consisting of an amino acid sequence of SEQ ID NO: 96 (as defined according to Kabat).

15H01

The amino acid sequence of the heavy chain variable region of antibody 15H01 is listed as SEQ ID NO: 45 and the amino acid sequence of the light chain variable region is listed as SEQ ID NO: 46.

In a specific aspect, the invention provides an antibody that binds essentially the same epitope or determinant as monoclonal antibodies 15H01; optionally the antibody comprises the hypervariable region of antibody 15H01. Optionally, the antibody 15H01 can be characterized by the amino acid sequences and/or nucleic acid sequences encoding it. In one aspect, the monoclonal antibody comprises the Fab or F(ab′)2 portion of 15H01. Also provided is a monoclonal antibody that comprises the heavy chain variable region of 15H01. According to one aspect, the monoclonal antibody comprises the three CDRs of the heavy chain variable region of 15H01. Also provided is a monoclonal antibody that further comprises the variable light chain variable region of 15H01 or one, two or three of the CDRs of the light chain variable region of 15H01. Optionally, any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 15H01 are fused to an immunoglobulin constant region of the human IgG type, optionally a human constant region.

In another aspect, the invention provides an antibody, wherein the antibody comprises: a HCDR1 region of 15H01 comprising an amino acid sequence as set forth in Table A-7, or a sequence of at least 4, 5, 6, 7, 8, 9 or 10 contiguous amino acids thereof, optionally wherein one or more of these amino acids may be substituted by a different amino acid; a HCDR2 region of 15H01 comprising an amino acid sequence as set forth in Table A-7, or a sequence of at least 4, 5, 6, 7, 8, 9 or 10 contiguous amino acids thereof, optionally wherein one or more of these amino acids may be substituted by a different amino acid; a HCDR3 region of 15H01 comprising an amino acid sequence as set forth in Table A-7, or a sequence of at least 4, 5, 6, 7, 8, 9 or 10 contiguous amino acids thereof, optionally wherein one or more of these amino acids may be substituted by a different amino acid; a LCDR1 region of 15H01 comprising an amino acid sequence as set forth in Table A-7, or a sequence of at least 4, 5, 6, 7, 8, 9 or 10 contiguous amino acids thereof, optionally wherein one or more of these amino acids may be substituted by a different amino acid; a LCDR2 region of 15H01 comprising an amino acid sequence as set forth in Table A-7, or a sequence of at least 4, 5, 6, 7, 8, 9 or 10 contiguous amino acids thereof, optionally wherein one or more of these amino acids may be substituted by a different amino acid; a LCDR3 region of 15H01 comprising an amino acid sequence as set forth in Table A-7, or a sequence of at least 4, 5, 6, 7, 8, 9 or 10 contiguous amino acids thereof, optionally wherein one or more of these amino acids may be deleted or substituted by a different amino acid. The HCDR1, 2, 3 and LCDR1, 2, 3 sequences can optionally be specified as all (or each, independently) being those of the Kabat numbering system (as indicated in Table A-7 for each CDR), those of the Chotia numbering system as indicated in Table A-7 for each CDR), those of the IMGT numbering system as indicated in Table A-7 for each CDR), or any other suitable numbering system.

In another aspect, the invention provides an antibody that binds specifically to STIM1_{Ins}, comprising (a) the hypervariable regions of the heavy chain variable region of SEQ ID NO: 45, optionally wherein one, two, three or more amino acids are substituted by a different amino acid; and (b) the hypervariable regions of the light chain variable region of SEQ ID NO: 46, optionally wherein one, two, three or more amino acids are substituted by a different amino acid.

In another aspect, the invention provides an antibody that specifically binds STIM1_{Ins}, comprising:
(a) a heavy chain CDR 1 amino acid sequence as shown in SEQ ID NO: 103, 106 or 109, optionally wherein one, two, three or more amino acids in a CDR may be substituted by a different amino acid;
(b) a heavy chain CDR 2 amino acid sequence as shown in SEQ ID NO: 104, 107 or 110, optionally wherein one, two, three or more amino acids in a CDR may be substituted by a different amino acid;
(c) a heavy chain CDR 3 amino acid sequence as shown in SEQ ID NO: 105 or 111, optionally wherein one, two, three or more amino acids in a CDR may be substituted by a different amino acid;
(d) a light chain CDR 1 amino acid sequence as shown in SEQ ID NO: 175 or 181, optionally wherein one, two, three or more amino acids in a CDR may be substituted by a different amino acid;
(e) a light chain CDR 2 amino acid sequence as shown in SEQ ID NO: 176 or 182, optionally wherein one, two, three or more amino acids in a CDR may be substituted by a different amino acid; and/or
(f) a light chain CDR 3 amino acid sequence as shown in SEQ ID NO: 177, optionally wherein one, two, three or more amino acids in a CDR may be substituted by a different amino acid.

In a specific aspect, the antibody or the antigen binding domain comprises:
a heavy chain variable domain comprising CDR 1, 2 and 3 of the heavy chain variable region of SEQ ID NO: 45 and (ii) a light chain variable domain comprising CDR 1, 2 and 3 of the light chain variable region of SEQ ID NO: 46;

or
a light chain comprising a light chain CDR1 (LCDR1) comprising or consisting of an amino acid sequence of SEQ ID NO: 175; a light chain CDR2 (LCDR2) comprising or consisting of an amino acid sequence of SEQ ID NO: 176; and a light chain CDR3 (LCDR3) comprising or consisting of an amino acid sequence of SEQ ID NO: 177; and a heavy chain comprising or consisting of a heavy chain CDR1 (HCDR1) comprising or consisting of an amino acid sequence of SEQ ID NO: 103; a heavy chain CDR2 (HCDR2) comprising an amino acid sequence of SEQ ID NO: 104; and a heavy chain CDR3 (HCDR3) comprising or consisting of an amino acid sequence of SEQ ID NO: 105 (as defined according to Kabat).

16A08

The amino acid sequence of the heavy chain variable region of antibody 16A08 is listed as SEQ ID NO: 47 and the amino acid sequence of the light chain variable region is listed as SEQ ID NO: 48.

In a specific aspect, the invention provides an antibody that binds essentially the same epitope or determinant as monoclonal antibodies 16A08; optionally the antibody comprises the hypervariable region of antibody 16A08. Optionally, the antibody 16A08 can be characterized by the amino acid sequences and/or nucleic acid sequences encoding it. In one aspect, the monoclonal antibody comprises the Fab or F(ab′)2 portion of 16A08. Also provided is a monoclonal antibody that comprises the heavy chain variable region of 16A08. According to one aspect, the monoclonal antibody comprises the three CDRs of the heavy chain variable region of 16A08. Also provided is a monoclonal antibody that further comprises the variable light chain variable region of 16A08 or one, two or three of the CDRs of the light chain variable region of 16A08. Optionally, any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 16A08 are fused to an immunoglobulin constant region of the human IgG type, optionally a human constant region.

In another aspect, the invention provides an antibody, wherein the antibody comprises: a HCDR1 region of 16A08 comprising an amino acid sequence as set forth in Table A-8, or a sequence of at least 4, 5, 6, 7, 8, 9 or 10 contiguous amino acids thereof, optionally wherein one or more of these amino acids may be substituted by a different amino acid; a HCDR2 region of 16A08 comprising an amino acid sequence as set forth in Table A-8, or a sequence of at least 4, 5, 6, 7, 8, 9 or 10 contiguous amino acids thereof, optionally wherein one or more of these amino acids may be substituted by a different amino acid; a HCDR3 region of 16A08 comprising an amino acid sequence as set forth in Table A-8, or a sequence of at least 4, 5, 6, 7, 8, 9 or 10 contiguous amino acids thereof, optionally wherein one or more of these amino acids may be substituted by a different amino acid; a LCDR1 region of 16A08 comprising an amino acid sequence as set forth in Table A-8, or a sequence of at least 4, 5, 6, 7, 8, 9 or 10 contiguous amino acids thereof, optionally wherein one or more of these amino acids may be substituted by a different amino acid; a LCDR2 region of 16A08 comprising an amino acid sequence as set forth in Table A-8, or a sequence of at least 4, 5, 6, 7, 8, 9 or 10 contiguous amino acids thereof, optionally wherein one or more of these amino acids may be substituted by a different amino acid; a LCDR3 region of 16A08 comprising an amino acid sequence as set forth in Table A-8, or a sequence of at least 4, 5, 6, 7, 8, 9 or 10 contiguous amino acids thereof, optionally wherein one or more of these amino acids may be deleted or substituted by a different amino acid. The HCDR1, 2, 3 and LCDR1, 2, 3 sequences can optionally be specified as all (or each, independently) being those of the Kabat numbering system (as indicated in Table A-8 for each CDR), those of the Chotia numbering system as indicated in Table A-8 for each CDR), those of the IMGT numbering system as indicated in Table A-8 for each CDR), or any other suitable numbering system.

In another aspect, the invention provides an antibody that binds specifically to STIM1_{Ins}, comprising (a) the hypervariable regions of the heavy chain variable region of SEQ ID NO: 47, optionally wherein one, two, three or more amino acids are substituted by a different amino acid; and (b) the hypervariable regions of the light chain variable region of SEQ ID NO: 48, optionally wherein one, two, three or more amino acids are substituted by a different amino acid.

In another aspect, the invention provides an antibody that specifically binds STIM1_{Ins}, comprising:
(a) a heavy chain CDR 1 amino acid sequence as shown in SEQ ID NO: 112, 115 or 118, optionally wherein one, two, three or more amino acids in a CDR may be substituted by a different amino acid;
(b) a heavy chain CDR 2 amino acid sequence as shown in SEQ ID NO: 113, 116 or 119, optionally wherein one, two, three or more amino acids in a CDR may be substituted by a different amino acid;
(c) a heavy chain CDR 3 amino acid sequence as shown in SEQ ID NO: 114 or 120, optionally wherein one, two, three or more amino acids in a CDR may be substituted by a different amino acid;
(d) a light chain CDR 1 amino acid sequence as shown in SEQ ID NO: 184 or 190, optionally wherein one, two, three or more amino acids in a CDR may be substituted by a different amino acid;
(e) a light chain CDR 2 amino acid sequence as shown in SEQ ID NO: 185 or 191, optionally wherein one, two, three or more amino acids in a CDR may be substituted by a different amino acid; and/or
(f) a light chain CDR 3 amino acid sequence as shown in SEQ ID NO: 186, optionally wherein one, two, three or more amino acids in a CDR may be substituted by a different amino acid.

In a specific aspect, the antibody or the antigen binding domain comprises:
a heavy chain variable domain comprising CDR 1, 2 and 3 of the heavy chain variable region of SEQ ID NO: 47 and (ii) a light chain variable domain comprising CDR 1, 2 and 3 of the light chain variable region of SEQ ID NO: 48;

or
a light chain comprising a light chain CDR1 (LCDR1) comprising or consisting of an amino acid sequence of SEQ ID NO: 184; a light chain CDR2 (LCDR2) comprising or consisting of an amino acid sequence of SEQ ID NO: 185; and a light chain CDR3 (LCDR3) comprising or consisting of an amino acid sequence of SEQ ID NO: 186; and a heavy chain comprising or consisting of a heavy chain CDR1 (HCDR1) comprising or consisting of an amino acid sequence of SEQ ID NO: 112; a heavy chain CDR2 (HCDR2) comprising an amino acid sequence of SEQ ID NO: 113; and a heavy chain CDR3 (HCDR3) comprising or consisting of an amino acid sequence of SEQ ID NO: 114 (as defined according to Kabat).

In another aspect, the present invention relates to an antibody which competes with an antibody selected from the group of 1E05, 1F02, 2F12, 4B05, 8E11, 15G09, 15H01 and 16A08 for binding to a polypeptide comprising the amino acid sequence of SEQ ID NO: 1 or a fragment thereof, said fragment comprising a sequence of SEQ ID NO: 3.

In certain embodiments, one pre-mixes the control antibodies (1E05, 1F02, 2F12, 4B05, 8E11, 15G09, 15H01 or 16A08, for example) with varying amounts of the test antibodies (e.g., about 1:10 or about 1:100) for a period of time prior to applying to the STIM1_{Ins} antigen sample. In other embodiments, the control and varying amounts of test antibodies can simply be admixed during exposure to the STIM1_{Ins} antigen sample. As long as one can distinguish bound from free antibodies (e. g., by using separation or washing techniques to eliminate unbound antibodies) and 1E05, 1F02, 2F12, 4B05, 8E11, 15G09, 15H01 or 16A08) from the test antibodies (e. g., by using species-specific or isotype-specific secondary antibodies or by specifically labeling 1E05, 1F02, 2F12, 4B05, 8E11, 15G09, 15H01 or 16A08 with a detectable label) one can determine if the test antibodies reduce the binding of 1E05, 1F02, 2F12, 4B05, 8E11, 15G09, 15H01 or 16A08 to the antigens. The binding of the (labeled) control antibodies in the absence of a completely irrelevant antibody can serve as the control high value. The control low value can be obtained by incubating the labeled antibodies with unlabelled antibodies of exactly the same type, where competition would occur and reduce binding of the labeled antibodies. In a test assay, a significant reduction in labeled antibody reactivity in the presence of a test antibody is indicative of a test antibody that may recognize substantially the same epitope, i.e., one that “cross-reacts” or competes with the labeled antibody. Any test antibody that reduces the binding of reference antibody to STIM1_{Ins} antigens by at least about 50%, such as at least about 60%, or more preferably at least about 80% or 90% (e. g., about 65-100%), at any ratio of anti-STIM1_{Ins} antibody:test antibody between about 1:10 and about 1:100 is considered to be an antibody that competes with the reference antibody. Preferably, such test antibody will reduce the binding of the reference antibody (e.g., 1E05, 1F02, 2F12, 4B05, 8E11, 15G09, 15H01 or 16A08) to the STIM1ᵢₙₛ antigen by at least about 90% (e.g., about 95%). Competition can also be assessed by, for example, a flow cytometry test or BIACORE chip analysis.

In another aspect, the present invention relates to an antibody that binds substantially the same epitope as antibody 1E05, 1F02, 2F12, 4B05, 8E11, 15G09, 15H01 or 16A08. In one aspect, the antibody binds to an epitope of STIM1ᵢₙₛ that at least partially overlaps with, or includes at least one residue in, the epitope bound by antibody 1E05, 1F02, 2F12, 4B05, 8E11, 15G09, 15H01 or 16A08. The residues bound by the antibody can be specified as being present on the surface of the of the STIM1_{Ins} polypeptide, e.g. in a STIM1_{Ins} polypeptide expressed on the surface of a cell.

The sequences of the CDRs, according to IMGT, Kabat and Chothia definitions systems, have been summarized in Table A-1, A-2, A-3, A-4, A-5, A-6, A-7 and A-8 below. The sequences of the variable regions of the antibodies according to the invention are listed in Table B below.

TABLE A-1
Antibody 1E05

| | SEQ | | SEQ | | SEQ | |
|---|---|---|---|---|---|---|
| CDR | ID | HCDR1 | ID | HCDR2 | ID | HCDR3 |
| Kabat | 49 | DYYMH | 50 | WIDPENGN | 51 | DGFDY |
| | | | | TIYDPKFQ | | |
| | | | | G | | |
| Chotia | 52 | GFNIKDY | 53 | DPENGN | 54 | DGFDY |
| IMPGT | 55 | GFNIKDYY | 56 | IDPENGNT | 57 | AMDGFDY |
| | SEQ | | SEQ | | SEQ | |
| CDR | ID | LCDR1 | ID | LCDR2 | ID | LCDR3 |
| Kabat | 121 | KSSQSLLD | 122 | LVSKLDS | 123 | WQGTHFPLT |
| | | SDGKTYLN | | | | |
| Chotia | 124 | KSSQSLLD | 125 | LVSKLDS | 126 | WQGTHFPLT |
| | | SDGKTYLN | | | | |
| IMPGT | 127 | QSLLDSDG | 128 | LVS | 129 | WQGTHFPLT |
| | | KTY | | | | |

TABLE A-2
Antibody 1F02

| | SEQ | | SEQ | | SEQ | |
|---|---|---|---|---|---|---|
| CDR | ID | HCDR1 | ID | HCDR2 | ID | HCDR3 |
| Kabat | 58 | NYGMY | 59 | WINTKTGE | 60 | LGGGYFDV |
| | | | | PTYAEEFK | | |
| | | | | G | | |
| Chotia | 61 | GYTFTNY | 62 | NTKTGE | 63 | LGGGYFDV |
| IMPGT | 64 | GYTFTNYG | 65 | INTKTGEP | 66 | ANLGGGYFDV |
| | SEQ | | SEQ | | SEQ | |
| CDR | ID | LCDR1 | ID | LCDR2 | ID | LCDR3 |
| Kabat | 130 | KSSQSLLD | 131 | VVSKLDS | 132 | WQGTHFPLT |
| | | SDGKTYLN | | | | |
| Chotia | 133 | KSSQSLLD | 134 | VVSKLDS | 135 | WQGTHFPLT |
| | | SDGKTYLN | | | | |
| IMPGT | 136 | QSLLDSDG | 137 | WS | 138 | WQGTHFPLT |
| | | KTY | | | | |

TABLE A-3
Antibody 2F12

| | SEQ | | SEQ | | SEQ | |
|---|---|---|---|---|---|---|
| CDR | ID | HCDR1 | ID | HCDR2 | ID | HCDR3 |
| Kabat | 67 | DYAMH | 68 | WINTETGE | 69 | PTGTGDF |
| | | | | PTYADDFK | | |
| | | | | G | | |
| Chotia | 70 | VYTFTDY | 71 | NTETGE | 72 | PTGTGDF |
| IMPGT | 73 | VYTFTDYA | 74 | INTETGEP | 75 | VSPTGTGDF |
| | SEQ | | SEQ | | SEQ | |
| CDR | ID | LCDR1 | ID | LCDR2 | ID | LCDR3 |
| Kabat | 139 | KSSQSLLD | 140 | LVSKLDS | 141 | WQGTHFPLT |
| | | SDGKTYLN | | | | |
| Chotia | 142 | KSSQSLLD | 143 | LVSKLDS | 144 | WQGTHFPLT |
| | | SDGKTYLN | | | | |
| IMPGT | 145 | QSLLDSDG | 146 | LVS | 147 | WQGTHFPLT |
| | | KTY | | | | |

TABLE A-4
Antibody 4B05

| | SEQ | | SEQ | | SEQ | |
|---|---|---|---|---|---|---|
| CDR | ID | HCDR1 | ID | HCDR2 | ID | HCDR3 |
| Kabat | 76 | SYGMS | 77 | TITGGDGF | 78 | SDY |
| | | | | TYYPDSVK | | |
| | | | | G | | |
| Chotia | 79 | GFTFNSY | 80 | TGGDGF | 81 | SDY |
| IMPGT | 82 | GFTFNSYG | 83 | ITGGDGFT | 84 | TRSDY |
| | SEQ | | SEQ | | SEQ | |
| CDR | ID | LCDR1 | ID | LCDR2 | ID | LCDR3 |
| Kabat | 148 | KSSRSLLD | 149 | LVSKLDS | 150 | WQGKHSPWT |
| | | TDGETYLN | | | | |
| Chotia | 151 | KSSRSLLD | 152 | LVSKLDS | 153 | WQGKHSPWT |
| | | TDGETYLN | | | | |
| IMPGT | 154 | RSLLDTDG | 155 | LVS | 156 | WQGKHSPWT |
| | | ETY | | | | |

TABLE A-5
Antibody 8E11

| | SEQ | | SEQ | | SEQ | |
|---|---|---|---|---|---|---|
| CDR | ID | HCDR1 | ID | HCDR2 | ID | HCDR3 |
| Kabat | 85 | DSVIT | 86 | EIYPGSGS | 87 | NYNGMDY |
| | | | | IYYNEKFKG | | |
| Chotia | 88 | GYIFSDS | 89 | YPGSGS | 90 | NYNGMDY |
| IMPGT | 91 | GYIFSDSV | 92 | IYPGSGSI | 93 | ANNYNGMDY |
| | SEQ | | SEQ | | SEQ | |
| CDR | ID | LCDR1 | ID | LCDR2 | ID | LCDR3 |
| Kabat | 157 | KSSQSLLD | 158 | LVSKLDS | 159 | VQGTHFPFT |
| | | SSGKTYLN | | | | |
| Chotia | 160 | KSSQSLLD | 161 | LVSKLDS | 162 | VQGTHFPFT |
| | | SSGKTYLN | | | | |
| IMPGT | 163 | QSLLDSSG | 164 | LVS | 165 | VQGTHFPFT |
| | | KTY | | | | |

TABLE A-6
Antibody 15G09

| | SEQ | | SEQ | | SEQ | |
|---|---|---|---|---|---|---|
| CDR | ID | HCDR1 | ID | HCDR2 | ID | HCDR3 |
| Kabat | 94 | NFGMN | 95 | WINTNTG | 96 | PTGKGDY |
| | | | | EPTYADD | | |
| | | | | FKG | | |
| Chotia | 97 | GYIFSNF | 98 | NTNTGE | 99 | PTGKGDY |
| IMPGT | 100 | GYIFSNFG | 101 | INTNTGEP | 102 | VRPTGKGDY |
| | SEQ | | SEQ | | SEQ | |
| CDR | ID | LCDR1 | ID | LCDR2 | ID | LCDR3 |
| Kabat | 166 | KSSQSLLD | 167 | LVSKLDS | 168 | WQGTHFPQT |
| | | SDGKTYLN | | | | |
| Chotia | 169 | KSSQSLLD | 170 | LVSKLDS | 171 | WQGTHFPQT |
| | | SDGKTYLN | | | | |
| IMPGT | 172 | QSLLDSDG | 173 | LVS | 174 | WQGTHFPQT |
| | | KTY | | | | |

TABLE A-7
Antibody 15H01

| | SEQ | | SEQ | | SEQ | |
|---|---|---|---|---|---|---|
| CDR | ID | HCDR1 | ID | HCDR2 | ID | HCDR3 |
| Kabat | 103 | DYGMD | 104 | NINPNNGG | 105 | PYDGGDY |
| | | | | TIYNQKFKG | | |
| Chotia | 106 | GYTFTDY | 107 | NPNNGG | 108 | PYDGGDY |
| IMPGT | 109 | GYTFTDYG | 110 | INPNNGGT | 111 | ASPYDGGDY |
| | SEQ | | SEQ | | SEQ | |
| CDR | ID | LCDR1 | ID | LCDR2 | ID | LCDR3 |
| Kabat | 175 | KSSQSLLY | 176 | SVSKLDS | 177 | VQGTHFPWT |
| | | SNGKTYLN | | | | |
| Chotia | 178 | KSSQSLLY | 179 | SVSKLDS | 180 | VQGTHFPWT |
| | | SNGKTYLN | | | | |
| IMPGT | 181 | QSLLYSNG | 182 | SVS | 183 | VQGTHFPWT |
| | | KTY | | | | |

TABLE A-8
Antibody 16A08

| | SEQ | | SEQ | | SEQ | |
|---|---|---|---|---|---|---|
| CDR | ID | HCDR1 | ID | HCDR2 | ID | HCDR3 |
| Kabat | 112 | NYGMH | 113 | WINTKTG | 114 | AYGDYPY |
| | | | | EPRFADD | | |
| | | | | FKG | | |
| Chotia | 115 | GYTFTNY | 116 | NTKTGE | 117 | AYGDYPY |
| IMPGT | 118 | GYTFTNYG | 119 | INTKTGEP | 120 | SRAYGDYPY |
| | SEQ | | SEQ | | SEQ | |
| CDR | ID | LCDR1 | ID | LCDR2 | ID | LCDR3 |
| Kabat | 184 | KSSQSLLY | 185 | LVSKLDS | 186 | WQGTHFPQT |
| | | SDGKTYLN | | | | |
| Chotia | 187 | KSSQSLLY | 188 | LVSKLDS | 189 | WQGTHFPQT |
| | | SDGKTYLN | | | | |
| IMPGT | 190 | QSLLYSDG | 191 | LVS | 192 | WQGTHFPQT |
| | | KTY | | | | |

TABLE B

| | SEQ ID | |
|---|---|---|
| Ab | NO | Amino Acid Sequence |
| 1E05 VH | 33 | QVQLQQSGAELVRPGALVKLSCKASGFNIKDYYMHWVKQRPEQGLEWIGWIDPENG |
| | | NTIYDPKFQGKASITADTSSNTAYLQLSSLTSEDTAVYYCAMDGFDYWGQGTTLTVS |
| | | S |
| 1E05 VL | 34 | DVVMTQTPLTLSVTIGQPASISCKSSQSLLDSDGKTYLNWLLQRPGQSPKRLIYLVSK |
| | | LDSGVPGRFTASGSGTDFTLRISRVETEDLGVYYCWQGTHFPLTFGTGTKLELK |
| 1F02 VH | 35 | QIQLVQSGPELKKPGETVKISCKASGYTFTNYGMYWVKQAPGKGLKWMAWINTKTG |
| | | EPTYAEEFKGRFALSLETSANTAYLQINNLKNEDTASYFCANLGGGYFDVWGAGTTV |
| | | TVSS |
| 1F02 VL | 36 | DVVMTQTPLSLSVTIGQTASISCKSSQSLLDSDGKTYLNWLLQRPGQSPKRLIYVVSK |
| | | LDSGVPDRFTGSGSGTDFTLKISRVEAEDLGVYYCWQGTHFPLTFGAGTKLELK |
| 2F12 VH | 37 | QIQLVQSGPELKKPGETVKISCKSSVYTFTDYAMHWVKQAPGKGLKWMGWINTETG |
| | | EPTYADDFKGRFAFSLETSVSTAYLHINNLKNEDTATFFCVSPTGTGDFWGQGTTLT |
| | | VSS |
| 2F12 VL | 38 | DAVMTQSPLTLSVTIGQPASMSCKSSQSLLDSDGKTYLNWLLQRPGQSPKRLIYLVS |
| | | KLDSGVPDRFTGSGSGTDFTLKISRVEAEDLGVYYCWQGTHFPLTFGAGTKLEIK |
| 4B05 VH | 39 | EVQLVESGGDLVKPGGSLKLSCAASGFTFNSYGMSWVRQTPDKRLEWVATITGGDG |
| | | FTYYPDSVKGRFTISRDNAKNTLYLQMSSLKSEDTAIYYCTRSDYWGQGTTLTVSS |
| 4B05 VL | 40 | DVVMTQTPLTLSVTIGQPASISCKSSRSLLDTDGETYLNWLLQRPGQSPRRLIYLVSK |
| | | LDSGVPDRFTGSGSGTDFTLKISRVEAEDLGVYYCWQGKHSPWTFGGGTKLEIK |
| 8E11 VH | 41 | QVQLKQSGPELVKPGASVKMSCKASGYIFSDSVITWVKQRIGQGLEWIGEIYPGSGSI |
| | | YYNEKFKGKATLTADKSSNTVYIQLGSLTSEDSAVYFCANNYNGMDYWGQGTSVTV |
| | | SS |
| 8E11 VL | 42 | DVVMTQTPLTLSVAIGQPASISCKSSQSLLDSSGKTYLNWLLQRPGQSPKRLIYLVSK |
| | | LDSGVPDRFTGSGSGTDFTLKISRVEAEDLGIYYCVQGTHFPFTFGSGTKLEIK |
| 15G09 VH | 43 | QIQLVQSGPELKKPGETVRISCKASGYIFSNFGMNWVKQAPGKGLRWMGWINTNTG |
| | | EPTYADDFKGRFVFSLEASAHTAYLQINNVKNEDMATYFCVRPTGKGDYWGRGTTL |
| | | TVSS |
| 15G09 VL | 44 | DVVMTQTPLTLSVTIGQPASISCKSSQSLLDSDGKTYLNWLLQRPGQSPKRLIYLVSK |
| | | LDSGVPDRFTGSGSGTDFTLKISRVEAEDLGVYYCWQGTHFPQTFGGGTKLEIK |
| 15H01 VH | 45 | EVQLQQSGPELVKPGASVKISCKASGYTFTDYGMDWVKQSHGKSLEWIGNINPNNG |
| | | GTIYNQKFKGKATLTVDKSSSTAYMELRSLTSEDTAVYYCASPYDGGDYWGQGTSV |
| | | TVSS |
| 15H01 VL | 46 | DVVMTQTPLTLSVTIGQPASISCKSSQSLLYSNGKTYLNWLLQRPGQSPKRLMYSVS |
| | | KLDSGVPDRFTGSGSGTDFTLKISRVEAEDLGVYYCVQGTHFPWTFGGGTKLEIK |
| 16A08 VH | 47 | QIQLVQSGPELKKPGETVKISCKASGYTFTNYGMHWVKQAPGKALKWMGWINTKTG |
| | | EPRFADDFKGRFAFSLETSANTAYLQINNLKNEDTATYFCSRAYGDYPYWGQGTTLT |
| | | VSS |
| 16A08 VL | 48 | DVVMTQTPLTLSVTIGQPASISCKSSQSLLYSDGKTYLNWLLQRPGQSPKRLIYLVSK |
| | | LDSGVPDRFTGSGSGTDFTLIISRVEAEDLGVYYCWQGTHFPQTFGGGTKLEIK |

The antibody or the fragment thereof can be an antibody selected from a fully human antibody, a humanized antibody, and a chimeric antibody. The antibody or the fragment thereof may comprise a constant domain selected from IgG1, IgG2, IgG3 and IgG4.

The fragment of the antibody is preferably a Fab fragment, a Fab′ fragment, a Fab′-SH fragment, a F(ab)2 fragment, a F(ab′)2 fragment, an Fv fragment, a Heavy chain Ig (a llama or camel Ig), a V_{HH} fragment, a single domain FV, and a single-chain antibody fragment.

In addition, the present invention also relates to a multispecific antibody (e.g., bispecific) formed from antibody fragments, in particular a multispecific or bispecific antibody including an antigen binding domain comprising any combination of CDRs disclosed herein, in particular the 6 CDRs of an antibody selected from the group consisting of 1E05, 1F02, 2F12, 4B05, 8E11, 15G09, 15H01 and 16A08. In one aspect, the present invention relates to a multispecific or bispecific antibody comprising a heavy chain variable domain and a light chain variable domain as defined any one of items (i) to (viii) herein. In a second aspect, it relates to a multispecific or bispecific antibody comprising a heavy chain variable domain and a light chain variable domain of an antibody selected from the group consisting of 1E05, 1F02, 2F12, 4B05, 8E11, 15G09, 15H01 and 16A08.

Optionally, the antibody can be a synthetic or semisynthetic antibody-derived molecule selected from a scFV, a dsFV, a minibody, a diabody, a triabody, a kappa body, an IgNAR; and a multispecific antibody.

Optionally, the antibody is in at least partially purified form or in essentially isolated form.

The present invention further relates to a chimeric antigen receptor (CAR) comprising an antigen binding domain comprising any combination of CDRs disclosed herein, in particular the 6 CDRs of an antibody selected from the group consisting of 1E05, 1F02, 2F12, 4B05, 8E11, 15G09, 15H01 and 16A08. In one aspect, the present invention relates to a CAR comprising a heavy chain variable domain and a light chain variable domain as defined any one of items (i) to (viii) herein. In a second aspect, it relates to a CAR comprising a heavy chain variable domain and a light chain variable domain of an antibody selected from the group consisting of 1E05, 1F02, 2F12, 4B05, 8E11, 15G09, 15H01 and 16A08. Optionally, the antigen binding domain is a scFv.

Optionally, the CAR comprises an antigen binding domain as defined herein, a transmembrane domain and an intracellular domain comprising a cytoplasmic signaling domain and optionally at least one costimulatory domain. The CAR may further comprise a signal peptide and/or a hinge region.

The intracellular domain may comprise a CD3 zeta signaling domain and optionally at least one costimulatory domain(s) selected from CD28, 41BB, CD134, CD2, CD7, ICOS, OX40, CD149, DAP10, CD30, CD40, CD70, CD134, PD-1, DAP10, ICAM-1, LFA-1, NKG2D, GITR, TLR2, IL2-R, IL7r6, IL21-R, NKp30, NKp44, CD27, CD137 and DNAM-1 costimulatory domains, preferably the two costimulatory domains are 41BB and CD28 costimulatory domains.

The transmembrane domain can be selected from transmembrane domain of CD8, CD28, CD3E, CD45, CD4, CD5, CD9, CD16, CD22, CD33, CD37, CD64, CD80, CD86, CD134, CD137, or CD154, preferably CD28, CD3 and CD8 transmembrane domains, preferably the transmembrane domain is a CD28 transmembrane domain.

The hinge region can comprise a hinge region of Ig4, CD8, CD28, CD137, or combinations thereof, wildtype or mutants.

The present invention relates to a cell comprising a CAR as defined above. Preferably, the cell is selected from a group consisting of a T cell, CD4⁺ T cell, CD8⁺ T cell, B cell, NK cell, NKT cell, monocyte and dendritic cell, preferably the cell being a T cell, a B cell or a NK cell.

The present invention further relates to an antibody specific to the isoform STIM1ins which is covalently linked to a detectable label or a drug.

The antibody can be labeled with a label selected from the group consisting in a radiolabel, an enzyme label, a fluorescent label, a biotin-avidin label, a chemoluminescent label, and the like. The antibody a can be labeled by standard labeling techniques well known by the man skilled in the art and labeled antibodies can be visualized using known methods. In particular, labels generally provide signals detectable by fluorescence, chemoluminescence, radioactivity, colorimetry, mass spectrometry, X-ray diffraction or absorption, magnetism, enzymatic activity, or the like.

The drug can be a cytotoxic drug. As used herein, the term “cytotoxic drug” refers to a molecule that when entering in contact with a cell, eventually upon internalization into the cell, alters a cell function (e.g. cell growth and/or proliferation and/or differentiation and/or metabolism such as protein and/or DNA synthesis) in a detrimental way or leads to cell death. As used herein, the term “cytotoxic drug” encompasses toxins, in particular cytotoxins.

The cytotoxic drug according to the invention may be selected from the group consisting of dolastatins such as dolastin 10, dolastin 15, auristatin E, auristatin EB (AEB), auristatin EFP (AEFP), monomethyl auristatin F (MMAF), monomethylauristatin-D (MMAD), monomethyl auristatin E (MMAE), and 5-benzoylvaleric acid-AE ester (AEVB), maytansines such ansamitocin, mertansine (also called emtansine or DM1) and ravtansine (also called soravtansine or DM4), antracyclins such as daunorubicin, epirubicin, pirarubicin, idarubicin, zorubicin, cerubidin, aclarubicin, adriblastin, doxorubicin, mitoxantrone, daunoxome, nemorubicin and PNU-159682, calicheamicins such as calicheamicin beta 1Br, calicheamicin gamma 1Br, calicheamicin alpha 2I, calicheamicin alpha 3I, calicheamicin beta 1I, calicheamicin gamma 1L, calicheamicin delta 1I and ozogamicin, esperamicins such as esperamicin A1, neocarzinostatins, bleomycin, duocarymycins such as CC-1065 and duocarmycin A, pyrrolobenzodiazepines such as anthramycin, abbeymycin, chicamycin, DC-81, mazethramycin, neothramycins A and B, porothramycin prothracarcin, sibanomicin (DC-102), sibiromycin and tomamycin, pyrrolobenzodiazepine dimers (or PBD), indolino-benzodiazepines, indolino-benzodiazepine dimers, α-amanitins, abraxane, actinomycin, aldesleukin, altretamine, alitretinoin, amsacrine, anastrozole, arsenic, asparaginase, azacitidine, azathioprine, bexarotene, bendamustine, bicalutamide, bortezomib, busulfan, capecitabine, carboplatin, carmustine, chlorambucil, cisplatin, cladribine, clofarabine, cyclophosphamide, cytarabine, chloramphenicol, ciclosporin, cidofovir, coal tar containing products, colchicine, dacarbazine, dactinomycin, danazol, dasatinib, diethylstilbestrol, dinoprostone, dithranol, dutasteride, dexrazoxane, docetaxel, doxifluridine, erlotinib, estramustine, etoposide, exemestane, finasteride, flutamide, floxuridine, flucytosine, fludarabine, fluorouracil, ganciclovir, gefitinib, gemcitabine, goserelin, hydroxyurea, hydroxycarbamide, ifosfamide, irinotecan, imatinib, lenalidomide, leflunomide, letrozole, leuprorelin acetate, lomustine, mechlorethamine, melphalan, mercaptopurine, methotrexate, mitomycin, mitotane, menotropins, mifepristone, nafarelin, nelarabin, nitrogen mustard, nitrosoureas, oxaliplatin, ozogamicin, paclitaxel, podophyllyn, pegasparaginase, pemetrexed, pentamidine, pentostatin, procarbazin, raloxifene, ribavarin, raltitrexed, rituximab, romidepsin, sorafenib, streptozocin, sunitinib, sirolimus, streptozocin, temozolomide, temsirolimus, teniposide, thalidomide, thioguanine, thiotepa, topotecan, tacrolimus, taxotere, tafluposide, toremifene, tretinoin, trifluridine, triptorelin, valganciclovir, valrubicin, vinblastine, vidaradine, vincristine, vindesine, vinorelbine, vemurafenib, vismodegib, vorinostat, zidovudine, vedotine, derivatives and combinations thereof.

The antibody can be linked to the drug (antibody-drug conjugate) through a linker. The linker may be cleavable or non-cleavable, preferably, the linker is cleavable. Examples of cleavable linkers according to the invention include, without limitations, disulfides, hydrazones and peptides. Examples of non-cleavable linkers according to the invention include, without limitations, thioethers.

In a particular aspect, the antibody is capable of blocking or reducing the activity of STIM1ᵢₙₛ. Preferably, the antibody has no effect or almost no effect on the activity of STIM1 of SEQ ID NO: 5. In particular, the activity could be measured by the quantification of the CCE or cell migration due to the STIM1 isoform tested, in particular of the isoform STIM1ᵢₙₛ.

In an alternative aspect, the antibody is capable of increasing the activity of STIM1ᵢₙₛ. Preferably, the antibody has no effect or almost no effect on the activity of STIM1 of SEQ ID NO: 5.

The antibody specific to the isoform STIM1ᵢₙₛ can be used as a detecting mean, especially in any of the methods disclosed herein in the context of the use of the isoform STIM1ᵢₙₛ as a biomarker, or as a drug in the context of the new therapeutic strategy based on the specific targeting of the isoform STIM1ᵢₙₛ.

Peptides from STIM1ᵢₙₛ

The present invention also relates to an isolated peptide of less than 100, 75, 50, 40, 30 or 20 amino acids in length and comprising an amino acid sequence disclosed in SEQ ID NO: 3. Optionally, the peptide may comprise the amino acid sequence disclosed in SEQ ID NO: 3 and at least 5, 10, 15 or 20 consecutive amino acids of SEQ ID NO: 1

This peptide can be bound to a detectable label or to a drug. The drug and label are as defined above.

In a particular embodiment, the peptide is not found in nature. The peptide is a non-natural peptide.

The N- and C-termini of the peptides described herein may be optionally protected against proteolysis. In a preferred embodiment, the N-terminus may be in the form of an acetyl group, and/or the C-terminus may be in the form of an amide group. In a preferred embodiment, the peptide has a free C-terminal end.

Alternatively or in addition, internal modifications of the peptides to be resistant to proteolysis are also envisioned, e.g. wherein at least a —CONH-peptide bond is modified and replaced by a (CH2NH) reduced bond, a (NHCO) retro-inverso bond, a (CH2-O) methylene-oxy bond, a (CH2-S) thiomethylene bond, a (CH2CH2) carba bond, a (CO—CH2) cetomethylene bond, a (CHOH—CH2) hydroxyethylene bond), a (N—N) bound, a E-alcene bond or also a CH═CH-bond.

For instance, the peptide may be modified by acetylation, acylation, amidation, cross-linking, cyclization, disulfide bond formation, formation of covalent cross-links, formation of cysteine, formation of pyroglutamate, formylation, gamma-carboxylation, glycosylation, GPI anchor formation, hydroxylation, iodination, methylation, myristylation, oxidation, phosphorylation, and the like.

The peptide according to the invention may comprise one or more amino acids which are rare amino acids in particular hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid; or synthetic amino acids in particular ornithine, norleucine, norvaline and cyclohexyl-alanine.

In a particular aspect, the peptide is capable of blocking or reducing the activity of STIM1ᵢₙₛ. Preferably, the peptide has no effect or almost no effect on the activity of STIM1 of SEQ ID NO: 5. In particular, the activity could be measured by the quantification of the CCE or cell migration due to the STIM1 isoform tested, in particular of the isoform STIM1ᵢₙₛ.

In an alternative aspect, the peptide is capable of increasing the activity of STIM1ᵢₙₛ. Preferably, the peptide has no effect or almost no effect on the activity of STIM1 of SEQ ID NO: 5.

The peptide can be used as a drug in the context of the new therapeutic strategy based on the specific targeting of the isoform STIM1ᵢₙₛ.

STIM1ᵢₙₛ as a Target for the Treatment of a Disease or Disorder

The inventors identified STIM1ᵢₙₛ as a therapeutic target. Therefore, the present invention relates to a pharmaceutical composition comprising a modulator specific to an isoform STIM1ᵢₙₛ or a molecule targeting specifically an isoform STIM1ᵢₙₛ, to a modulator specific to an isoform STIM1ᵢₙₛ or a molecule targeting specifically an isoform STIM1ᵢₙₛ for its use as a drug, to the use of a modulator specific to an isoform STIM1ᵢₙₛ or a molecule targeting specifically an isoform STIM1ᵢₙₛ for the manufacture of a medicine, or to a method for treating a disease in a subject comprising administering a therapeutic amount of a modulator specific to an isoform STIM1ᵢₙₛ or a molecule targeting specifically an isoform STIM1ᵢₙₛ to the subject. Optionally, the modulator can be an activator or an inhibitor. Preferably, the modulator is an inhibitor.

In particular, the disease or disorder to be treated is a cancer or a myelodysplastic syndrome. The cancer and myelodysplastic syndrome can be selected in the list provided in the present document. The disease or disorder to be treated can be any disease associated with a splicing defect, in particular with a mutation or alteration in a splice factor gene such as U2AF1, SRSF2, SUGP1 and SF3B1, preferably with a mutation or alteration in SF3B1 or SUGP1, more preferably a mutation in SF3B1.

In a first aspect, the modulator or molecule targeting specifically an isoform STIM1ᵢₙₛ is an antibody specific to the isoform STIM1ᵢₙₛ as defined above. Optionally, the antibody can be any antibody as disclosed above, including the antibody linked to a drug or a cytotoxic molecule. Optionally, the antibody can be a multispecific or bispecific antibody as disclosed above, including the multispecific or bispecific antibody linked to a drug or a cytotoxic molecule. Optionally, modulator or molecule targeting specifically an isoform STIM1ᵢₙₛ is a CAR as defined above or a cell comprising such a CAR. Optionally, the modulator can be an activator or an inhibitor. Preferably, the modulator is an inhibitor.

In a second aspect, the modulator or molecule targeting specifically an isoform STIM1ᵢₙₛ is a peptide comprising a fragment of the isoform STIM1ᵢₙₛ as defined above. Optionally, the peptide can be any peptide as disclosed above, including the antibody linked to a drug or a cytotoxic molecule. Optionally, the modulator can be an activator or an inhibitor. Preferably, the modulator is an inhibitor.

In another aspect, the modulator or molecule targeting specifically an isoform STIM1ᵢₙₛ is a molecule or an inhibitor reducing or blocking specifically the expression of an isoform of STIM1 of SEQ ID NO: 1. More particularly, the molecule or inhibitor has no effect or almost no effect on the expression of the isoform of STIM1 of SEQ ID NO: 5. The molecule or inhibitor can be a siRNA, shRNA, antisense such as gapmer, and aptamer specific to the mRNA encoding the isoform of STIM1 of SEQ ID NO: 1. The molecule or inhibitor will target the sequence corresponding to the introduced sequence, optionally with an overlap to the flanking sequence, on one side or on both side of the introduced sequence. In a particular aspect, the molecule or inhibitor targets a sequence comprising at least 10 consecutive nucleic acids of SEQ ID NO: 4, preferably at least 7 consecutive nucleic acids of the segments in positions 1-10 of SEQ ID NO: 4.

The person skilled in the art knows how to prepare such molecule or modulator.

Down-modulating or inhibitory nucleic acids include, without limitation, antisense molecules, aptamers, ribozymes, triplex forming molecules, and RNA interference (RNAi).

As used herein, an “ASO” or “antisense” refers to a modified single-stranded oligonucleotide comprising at least one region which is complementary to a target nucleic acid. ASOs are designed and commonly used to modulate the expression of their target nucleic acid, notably to knock down their target. The precise targeting of a specific nucleic acid, on which the selectivity of a knockdown strategy depends, relates to a balance between oligonucleotide length and complementarity rate toward the defined target. In addition, chemical modifications are generally required to confer an improvement in single-stranded oligonucleotide stability within cells, especially towards digestion by nucleases. Indeed, unmodified single-stranded oligonucleotides are too instable to use in cells. Notably, it is well known by the one skilled in the art that the nuclease resistance can be dramatically improved by modifying internucleotide linkage, e.g., by substituting phosphodiester bonds by phosphorothioate (PS) linkage. Furthermore, other chemical modifications can improve potency and selectivity of ASO by increasing binding affinity of ASOs for their target.

The ASO according to the invention is a single-stranded oligonucleotide comprising deoxyribonucleotides and/or ribonucleotides. In a first embodiment, the ASO according to the invention comprises ribonucleotides and deoxyribonucleotides, i. e. DNA or DNA-like nucleotides. In a second embodiment, the ASO according to the invention comprises only deoxyribonucleotides, i. e. DNA or DNA-like nucleotides. In a third embodiment, the ASO according to the invention comprises only ribonucleotides, i.e. RNA or RNA-like nucleotides.

According to their composition in nucleotides, i. e. ribonucleotides and deoxyribonucleotides or ribonucleotides exclusively, the ASO according to the invention can inhibit the expression of its target nucleic acid via different ways.

In a preferred embodiment, the ASO acts via RNase H mediated degradation. RNase H is a cellular enzyme which recognizes the duplex between DNA and RNA, and enzymatically cleaves the RNA molecule. Thus, ASO comprises a region that comprises DNA or DNA-like nucleotides complementary to the targeted STIM1ᵢₙₛ isoform which is responsible for RNAse H recruitment that leads to subsequent target nucleic acid cleavage.

Alternatively, the ASO according to the invention can inhibit the expression of STIM1ᵢₙₛ isoform by inhibiting the translation of STIM1ᵢₙₛ isoform. In another particular embodiment, the ASO according to the invention can inhibit the formation of mature RNAs of STIM1_{Ins} isoform by modulating the splicing of the pre RNAs of STIM1_{Ins} isoform.

The ASO according to the invention has an overall sequence length of at least 10 nucleotides, preferably at least 12 nucleotides, more preferably at least 16 nucleotides. In a preferred aspect, the ASO according to the invention has an overall sequence length of 10 to 30 nucleotides, more preferably of 12 to 30, 12 to 26, 13 to 24, 14 to 22, 14 to 17 or 16 to 18 nucleotides.

As the target of the ASO of the invention is STIM1_{Ins} isoform, the ASO comprises a nucleotide sequence which is complementary to STIM1_{Ins} isoform, in particular to a specific region of the STIM1_{Ins} isoform sequence, in particular of a sequence comprising at least 10 consecutive nucleic acids of SEQ ID NO: 4, preferably at least 7 consecutive nucleic acids of the segments in positions 1-10 of SEQ ID NO: 4.

The ASO according to the invention comprises a contiguous nucleotide sequence of 10 to 30 nucleotides in length that is complementary to a specific portion of the STIM1_{Ins} isoform, in particular of a sequence comprising at least 10 consecutive nucleic acids of SEQ ID NO: 4, preferably at least 7 consecutive nucleic acids of the segments in positions 1-10 of SEQ ID NO: 4. Preferably, the length of the ASO contiguous sequence is 12 to 30 contiguous nucleotides, alternatively 12 to 26, 13 to 24, 14 to 22, 14 to 17 or 16 contiguous nucleotides in length that is complementary to a specific portion of the STIM1_{Ins} isoform, in particular of a sequence comprising at least 10 consecutive nucleic acids of SEQ ID NO: 4, preferably at least 7 consecutive nucleic acids of the segments in positions 1-10 of SEQ ID NO: 4.

According to the invention, the ASO contiguous nucleotide sequence is at least 90, 95, 96, 97, 98, 99 or 100 percent complementary to a specific region of the STIM1_{Ins} isoform.

Optionally, the ASO contiguous nucleotide sequence may comprise 0, 1, 2 or 3 mismatches in complementarity toward the targeted specific region of the STIM1_{Ins} isoform. Preferably, the complementarity between the ASO contiguous nucleotide sequence and the STIM1_{Ins} isoform sequence comprises 0 to 2, 0 or 1, or more preferably 0 mismatch.

In particular embodiments, the ASO according to the invention comprises a contiguous nucleotide sequence of 10 to 30 nucleotides in length that is at least 90, 95, 96, 97, 98, 99 or 100 percent complementary to a specific region of the STIM1_{Ins} isoform.

The ASO according to the invention comprises chemical modifications that confer an improved stability of single-stranded oligonucleotides within cells, in particular modifications relative to internucleotide linkages.

In a preferred aspect, the ASO according to the invention comprises phosphorothioate linkages in place of the phosphodiester bonds. The phosphorothioate linkages are preferably localized at the ends of the ASO. More preferably, the ASO according to the invention comprises at least 10, 11, 12, 13, 14 or 15 phosphorothioate linkages in place of the phosphodiester bonds. In a particular aspect, all the internucleotide linkage of ASO are phosphorothioate linkages.

In a preferred aspect, the ASO according to the invention comprises chemical modifications allowing an increase in binding affinity of oligonucleotides for their target nucleic acid. Such nucleotide modifications can be, but are not limited to, the addition on ribose of group such as 2′-O-methyl (2′-O-Me), 2′-fluoro (2′-F), 2′-O-methoxyethyl (MOE), the introduction of methylene bridge between the 2′ and 4′ position of the ribose which define the “locked” nucleic acids (LNAs), or the introduction of constrained ethyl (cEt) bridged nucleic acid (BNA). Besides, phosphorodiamidate morpholinos (PMOs) represent another modification that enhances metabolic stability and affinity for ribonucleotides by replacing the sugar and backbone with a morpholino ring system. All these modifications are well known to the one skilled in the art and also contribute to enhance the oligonucleotide stability (see Watts et al. J Pathol. 2012 January; 226(2): 365-379; Seth et al. J Clin Invest. 2019; 129(3):915-925).

In particular embodiments, the ASO according to the invention comprises 2′-O-Me, 2′-F, MOE, LNA, cET or PMO modified nucleotides in the contiguous nucleotide sequence, preferably 2′-O-Me, 2′-F, MOE, or LNA modified nucleotides, more preferably MOE and/or LNA modified nucleotides. In a preferred embodiment, the ASO according to the invention comprises at least 1, 2, 3, 4, 5, 6, 7, 8, 9, 10, 11, or 12 LNA modified nucleotides. In another preferred embodiment, the ASO according to the invention comprises at least 1, 2, 3, 4, 5, 6, 7, 8, 9, 10, 11, or 12 MOE modified nucleotides. In a preferred aspect, the ASO according to the invention is capable of inhibiting the expression of the isoform of STIM1 of SEQ ID NO: 5 in the nucleus and in the cytoplasm. More particularly, the ASO is capable of decreasing the expression of the isoform of STIM1 of SEQ ID NO: 5 by 10, 20, 30, 40, 50, 60, 70, 80, 90, 95 or 99% in comparison of its expression in absence of the ASO.

In a preferred aspect, the ASO of the invention may be a gapmer, also termed gapmer oligonucleotide, antisense gapmer or gapmer designs. Classically, a gapmer comprises at least three distinct structural regions, namely a 5′ flank region (F), a gap region (G) and a 3′ flank region (F′). Besides, the F and F′ regions are composed of modified ribonucleotides (RNA*) whereas the G region is composed of deoxyribonucleotides, i. e. DNA or DNA-like nucleotides.

The antisense gapmers are commonly used to inhibit a target nucleic acid via RNase H mediated degradation. Thus, the G region that comprises DNA or DNA-like nucleotides is responsible for RNAse H recruitment that leads to subsequent target nucleic acid cleavage. In contrast, the F and F′ regions comprise contiguous ribonucleotide sequence that are complementary to a target nucleic acid, i. e. two distinct regions of their target, and are thus responsible for the binding specificity to this target.

The antisense gapmer according to the invention has an overall sequence length of at least 10 contiguous nucleotides, preferably at least 12 contiguous nucleotides, more preferably at least 16 contiguous nucleotides, that is complementary to a specific portion of the STIM1_{Ins} isoform, in particular of a sequence comprising at least 10 consecutive nucleic acids of SEQ ID NO: 4, preferably at least 7 consecutive nucleic acids of the segments in positions 1-10 of SEQ ID NO: 4. In a preferred aspect, the antisense gapmer according to the invention has an overall sequence length of 10 to 30 contiguous nucleotides, more preferably of 12 to 30, 12 to 26, 13 to 24, 14 to 22, 14 to 17 or 16 to 18 contiguous nucleotides, that is complementary to a specific portion of the STIM1_{Ins} isoform, in particular of a sequence comprising at least 10 consecutive nucleic acids of SEQ ID NO: 4, preferably at least 7 consecutive nucleic acids of the segments in positions 1-10 of SEQ ID NO: 4.

In particular embodiments, the gapmer according to the invention consists or comprises a contiguous nucleotide sequence that corresponds to the following classical gapmer formula:

5′-F(RNA*)-G(DNA or DNA-like)-F′(RNA*)-3′

According to these embodiments of the invention, the F-G-F′ contiguous nucleotide sequence of the gapmer has an overall sequence length of at least 10 contiguous nucleotides, preferably at least 12 contiguous nucleotides, more preferably of 10 to 30 contiguous nucleotides, 12 to 30, 12 to 26, 13 to 24, 14 to 22, 14 to 17 or 16 to 18 contiguous nucleotides.

In a preferred embodiment, the ASO of the invention consists of or comprises a gapmer of formula 5′-F-G-F-3′, where region F and F′ region independently comprise or consist of 1 to 8 contiguous ribonucleotides, preferably 2 to 6 or 3 to 4 contiguous ribonucleotides, and G region comprises or consists of 6 to 16 deoxyribonucleotides, preferably 6 to 14, 6 to 12, 6 to 10, 6 to 8, 10 to 15, 10 to 14, or 11 to 15 contiguous deoxyribonucleotides.

As for the other ASOs, gapmer nucleotides are necessarily modified to confer an improved stability to the oligonucleotide within cells. Such modifications are notably relative to the introduction of internucleotides linkages. For instance, it is notably known to the one skilled in the art that the substitution of phosphodiester bonds by phosphorothioate PS linkages.

In a preferred aspect, the antisense gapmer according to the invention comprises phosphorothioate linkages in place of the phosphodiester bonds. The phosphorothioate linkages are preferably localized at the ends of the gapmer. More preferably, the antisense gapmer according to the invention comprises at least 10, 11, 12, 13, 14 or 15 phosphorothioate linkages in place of the phosphodiester bonds. In a particular aspect, all the internucleotide linkage of the gapmer are phosphorothioate linkages.

The F and F′ regions usually comprise modified ribonucleotides that enhance the gapmer binding affinity to its target nucleic acid and thus ensure a better selectivity for the knockdown strategy. In particular embodiments, the antisense gapmer according to the invention comprises 2′-O-Me, 2′-F, MOE, LNA, cET or PMO modified ribonucleotides in the F and F′ regions, preferably 2′-O-Me, 2′-F, MOE, or LNA modified nucleotides, more preferably MOE and/or LNA modified ribonucleotides.

In an aspect of the invention the antisense gapmer consists of or comprises a gapmer of formula 5′-F-G-F′-3′, where region F and F′ independently comprise or consist of 1 to 8, preferably 2 to 6 or 3 to 4 2′ sugar modified nucleotides, wherein there is at least one 2′ sugar modified nucleotide positioned at the 3′ end of region F (adjacent to a deoxynucleotide of region G), and at least one 2′ sugar modified nucleoside positioned at the 5′ end of region F′ (positioned adjacent to a deoxynucleotide of region G), and G is a region between 6 and 16 nucleosides which are capable of recruiting RNaseH, preferably a region of 6 to 16, 10 to 15, 10 to 14, such as 11 to 15, or 13 to 15 contiguous deoxynucleotides.

In a preferred embodiment, the antisense gapmer according to the invention comprises at least 1, 2, 3, 4, 5, 6, 7, 8, 9, 10, 11, or 12 LNA modified nucleotides. In another preferred embodiment, the antisense gapmer according to the invention comprises at least 1, 2, 3, 4, 5, 6, 7, 8, 9, 10, 11, or 12 MOE modified nucleotides.

In a preferred aspect, the antisense gapmer according to the invention is capable of inhibiting the expression of the isoform of STIM1 of SEQ ID NO: 5 in the nucleus, optionally in the nucleus and in the cytoplasm. More particularly, the antisense gapmer is capable of decreasing the expression of the STIM1_{In} isoform by at least 10, 20, 30, 40, 50, 60, 70, 80, 90 or 100% in comparison of its expression in absence of the gapmer.

In certain embodiments, the antisense gapmer according to the invention can be an LNA gapmer, a MOE gapmer, a mixed wing gapmer or an alternating flank gapmer.

In a particular aspect, the gapmer is a LNA gapmer. The term “LNA gapmer” refers to a gapmer oligonucleotide wherein at least one of the affinities enhancing modified nucleotides is an LNA nucleotide, i. e. a nucleotide comprising a methylene bridge between the 2′ and 4′ position of the ribose.

In a particular aspect, the gapmer is a MOE gapmer. The term “MOE gapmer” refers to a gapmer wherein at least one of the affinity enhancing modified nucleotides is an MOE nucleotide, i. e. a nucleotide comprising the addition of a group methoxyethyl (MOE) group at the 2′-O position.

In particular embodiments, the gapmer according to the invention consists or comprises a contiguous nucleotide sequence that corresponds to variant of the classical gapmer formula. Indeed, gapmer of the invention can be a headmer, a tailmer, a mixed wing gapmer, an alternative flank gapmer, a gap-breaker gapmer (also called gap-dispupted gapmer) or comprise additional (D and D′) regions.

The terms “headmers” and “tailmers” refers to gapmer oligonucleotides capable of recruiting RNase H where one of the flank regions is missing, i.e., where only one of the ends of the oligonucleotide comprises affinity enhancing modified ribonucleotides. For headmers, the 3′ flank is missing {i.e., the 5′ flank comprises affinity enhancing modified nucleosides) and for tailmers, the 5′ flank is missing {i.e., the 3′ flank comprises affinity enhancing modified nucleosides). In particular embodiments, the gapmer according to the invention consists or comprises a contiguous nucleotide sequence that corresponds to the following headmer (i) or (ii) tailmer formulas:

5′-F(RNA*)-G(DNA or DNA-like)-3′ (i)

5′-G(DNA or DNA-like)-F′(RNA*)-3′ (ii)

The terms “mixed wing gapmer” refers to a LNA gapmer wherein one or both of region F and F′ comprise a 2′ substituted nucleotide, such as a 2′ substituted nucleotide independently selected from the group consisting of 2′-0-alkyl-RNA units, 2′-0-methyl-RNA units, 2′-amino-DNA units, 2′-fluoro-DNA units, 2′-alkoxy-RNA, MOE units, arabino nucleic acid (ANA) units and 2′-fluoro-ANA units, such as MOE nucleotides. In some embodiments, wherein at least one of region F and F′, or both region F and F′ comprise at least one LNA nucleotide, the remaining nucleotides of region F and F′ are independently selected from the group consisting of MOE and LNA. In some embodiments, wherein at least one of region F and F′, or both region F and F′ comprise at least two LNA nucleotides, the remaining nucleotides of region F and F′ are independently selected from the group consisting of MOE and LNA. In some mixed wing embodiments, one or both of region F and F′ may further comprise one or more deoxynucleotides. Some mixed wing gapmer designs are disclosed in WO2008/049085 and WO2012/109395.

In some gapmers, flank regions F and F′ may comprise both LNA and deoxynucleotides.

The terms “alternative flank gapmer” refers to a gapmer that comprise an alternating motif of LNA-DNA-LNA nucleotides. Alternative flank gapmers are thus LNA gapmer oligonucleotides where at least one of the flanks (F or F′) comprises deoxynucleotides in addition to the LNA nucleotide(s). In some embodiments, at least one of region F or F′, or both region F and F′, comprise both LNA nucleotides and deoxynucleotides. In such embodiments, the flanking region F or F′, or both F and F′ comprise at least three nucleotides, wherein the 5′ and 3′ most nucleotides of the F and/or F′ region are LNA nucleotides. Besides, an alternating flank region may comprise up to 3 contiguous deoxynucleotides, such as 1 to 2 or 1 or 2 or 3 contiguous deoxynucleotides.

The terms “gap breaker gapmer” or “gap-disrupted gapmer” refers to a gapmer wherein the G region comprise at least one 3′ endo modified nucleotides. There are numerous reports of the insertion of a modified nucleoside which confers a 3′ endo conformation into the gap region of gapmers, whilst retaining some RNase H recruitment capacity, see for example WO2013/022984.

Importantly, gap-breaker gapmer retain sufficient region of deoxynucleotides within the gap region to allow for RNase H recruitment. The ability of gap-breaker gapmers to recruit RNase H is typically sequence or even compound specific: see Rukov et al. 2015 Nucl. Acids Res. Vol. 43 pp. 8476-8487, which discloses gap-breaker gapmers recruiting RNase H, which in some instances provide a more specific cleavage of the target RNA.

In addition, modified nucleotides used within the gap region of gap-breaker oligonucleotides may for example be modified nucleosides which confer a 3′endo conformation, such as 2′-O-methyl (OMe) or MOE nucleotides, or even beta-D LNA nucleotides (the bridge between 2′ and 4′ of the ribose sugar ring of a nucleotide is in beta conformation), such as beta-D-oxy LNA or ScET nucleosides.

Some gap region of gap-breaker or gap-disrupted gapmers have a deoxynucleotide at the 5′ end of the gap (adjacent to the 3′ ribonucleotide of region F), and a deoxynucleotide at the 3′ end of the gap (adjacent to the 5′ ribonucleotide of region F′). Gapmers which comprise a disrupted gap typically retain a region of at least 3 or 4 contiguous deoxynucleotides at either the 5′ end or 3′ end of the gap region.

In some embodiments, region G of a gap disrupted gapmer comprises at least 6 deoxynucleotides, such as 6, 7, 8, 9, 10, 11, 12, 13, 14, 15 or 16 deoxynucleotides. Also, the deoxynucleotides may be contiguous or may optionally be interspersed with one or more modified nucleotides, with the proviso that the gap region G is capable of mediating an effective RNase H recruitment.

The gapmer according to the invention may in some embodiments comprise or consist of the contiguous nucleotide sequence of the classical gapmer formula, i.e. F-G-F′, and further comprising 5′ and/or 3′ nucleotides. The further 5′ and/or 3′ nucleotides may or may not be fully complementary to the target nucleic acid. Such further 5′ and/or 3′ nucleotides may be referred to as region D′ and D″ herein.

The addition of region D′ or D″ may be used for the purpose of joining the contiguous nucleotide sequence of the gapmer to a conjugate moiety or another functional group. When used for joining the gapmer sequence with a conjugate moiety, one peripheral region, i. e. D′ and/or D″, can serve as a biocleavable linker (described below). Alternatively, it may be used to provide exonuclease protection or for ease of synthesis or manufacture.

Region D′ and D″ can be attached to the 5′ end of region F (i), the 3′ end of region F′ (ii) or both (iii), respectively to generate designs of the following formulas:

D′-F-G-F″ (i)

F-G-F′-D″ (ii)

D′-F-G-F′-D″ (iii)

In this instance, the F-G-F′ is the gapmer portion of the oligonucleotide and region D′ or D″ constitute a separate part of the oligonucleotide. Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these.

As described above, the D′ or D′ region may serve as a nuclease susceptible biocleavable linker.

In some embodiments, the additional 5′ and/or 3′ end nucleotides are linked with phosphodiester linkages. Nucleotide based biocleavable linkers suitable for use as region D′ or D″ are notably disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide. Optionally, this gapmer is a LNA gapmer.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. Representative examples of how to make and use triplex forming molecules to bind a variety of different target molecules can be found in W099/32619, W099/53050, WO99/49029, WO01/34815 and U.S. Pat. Nos. 5,176,996, 5,645,985, 5,650,316, 5,683,874, 5,693,773, 5,834,185, 5,869,246, 5,874,566, and 5,962,426.

Gene expression can also be effectively silenced in a highly specific manner through RNA interference (RNAi). This silencing was originally observed with the addition of double stranded RNA (dsRNA) (Fire, A., et al., Nature, 391:806-11 (1998); Napoli, C., et al., Plant Cell, 2:279-89 (1990); Hannon, G. J., Nature, 418:244-51 (2002)). Once dsRNA enters a cell, it is cleaved by an RNase III-like enzyme, Dicer, into double stranded small interfering RNAs (siRNA) 21-23 nucleotides in length that contain 2 nucleotide overhangs on the 3′ ends (Elbashir, S. M., et al., Genes Dev., 15:188-200 (2001); Bernstein, E., et al., Nature, 409:363-6 (2001); Hammond, S. M., et al., Nature, 404:293-6 (2000)). In an ATP-dependent step, the siRNAs become integrated into a multi-subunit protein complex, commonly known as the RNAi induced silencing complex (RISC), which guides the siRNAs to the target RNA sequence (Nykanen, A., et al., Cell, 107:309-21 (2001)). At some point the siRNA duplex unwinds, and it appears that the antisense strand remains bound to RISC and directs degradation of the complementary mRNA sequence by a combination of endo and exonucleases (Martinez, J., et al., Cell, 110:563-74 (2002)).

Short RNAs can be introduced into the cell as either short hairpin RNAs (shRNAs) or small interfering RNA (siRNAs). In mammalian cells, both shRNAs and siRNAs are at least 10, 15 or 20 base pair (bp) long, typically 19, 20, 21, 22, 23, 24 or 25 bp long, and are designed to have complementarity to the target sequence. In the context of the present invention, they are designed to have complementarity to a specific portion of the STIM1_{Ins} isoform, in particular of a sequence comprising at least 10 consecutive nucleic acids of SEQ ID NO: 4, preferably at least 7 consecutive nucleic acids of the segments in positions 1-10 of SEQ ID NO: 4.

shRNAs are double stranded RNAs (dsRNAs) that contain a loop structure, and are processed into siRNA by the host enzyme DICER, an endo-RNase that contains RNase III domains (Bernstein, Caudy et al. 2001). siRNA are dsRNA containing two-nucleotide 3′ end overhangs and 5′-triphosphate extremities (Zamore, Tuschl et al. 2000; Elbashir, Lendeckel et al. 2001; Elbashir, Martinez et al. 2001). After processing, one strand of the siRNA will be loaded into the RISC (RNA-induced silencing complex). The siRNA will bind to its target based on complementarity.

As used herein, the term “siRNA” refers to a nucleic acid molecule capable of mediating RNA interference or gene silencing. Since siRNAs can inhibit the expression of target genes, they are provided for efficient gene knockdown method or gene therapy methods.

As used therein, the term “shRNA” refers to small hairpin RNA or short hairpin RNA and is used to silence genes via RNA interference. Usually, the shRNA may be introduced into the target cell using a vector. The shRNA hairpin structure may be cleaved by other substances in the cell to become siRNA.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to MR1) is selected. An aptamer with increased activity is selected, for example, using SELEX (Systematic Evolution of Ligands by Exponential enrichment). Suitable methods for producing and/or screening an aptamer library are described, for example, in Elloington and Szostak, Nature 346:818-22, 1990; U.S. Pat. Nos. 5,270,163; and/or 5,475,096.

The molecule or inhibitor according to the invention or the pharmaceutical composition according to the invention may be administered by any conventional route of administration. In particular, the molecule or inhibitor or the pharmaceutical composition of the invention can be administered by a topical, enteral, oral, parenteral, intranasal, intravenous, intra-arterial, intramuscular, intratumoral, subcutaneous or intraocular administration and the like. In particular, the molecule or inhibitor according to the invention or the pharmaceutical composition according to the invention can be formulated for a topical, enteral, oral, parenteral, intranasal, intravenous, intra-arterial, intramuscular, intratumoral, subcutaneous or intraocular administration and the like.

The pharmaceutical composition comprising the molecule or inhibitor is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

List of Cancers and Myelodysplastic Syndromes

In a particular aspect, the cancer or myelodysplastic syndrome is selected in the group consisting of a hematopoietic cancer or a solid tumor, more specifically selected from the group consisting of breast cancer, lung cancer, colon cancer, bladder cancer, leukemia such as chronic lymphocytic leukemia (CLL), acute myeloid leukemia (AML), and chronic myelomonocytic leukemia (CMML), endometrium cancer, melanoma (mucosal, uveal or cutaneous), prostate cancer, pancreas cancer, glioblastoma, rectal cancer, colorectal cancer, ovary cancer, liver cancer, lung cancer, thyroid cancer, testicular cancer, myeloma (multiple myeloma), cholangiocarcinoma, nervous system cancer, uterus cancer, peritoneum cancer, digestive cancer, lymphoma and kidney cancer and the myelodysplastic syndrome is selected from the group consisting of refractory anemia, refractory anemia with ring sideroblast (RARS), refractory cytopenia with multilineage dysplasia (RCMD), refractory anemia with excess blasts, refractory anemia with excess blasts in transformation, and chronic myelomonocytic leukemia, preferably the cancer or myelodysplastic syndrome being selected from myelodysplastic syndromes, acute myeloid leukemia (AML), chronic myelomonocytic leukemia (CMML), chronic lymphocytic leukemia (CLL), refractory anemia with ring sideroblast (RARS), refractory cytopenia with multilineage dysplasia (RCMD), uveal melanoma, malignant melanoma, breast cancer, prostate cancer and bladder cancer.

Indeed, several cancers or myelodysplastic syndromes have been characterized as having a splicing defect, in particular with a mutation or alteration in a splice factor gene such as U2AF1, SRSF2, SUGP1 and SF3B1, preferably with a mutation or alteration in SF3B1 or SUGP1, more preferably a mutation in SF3B1. This is clearly known in the art as illustrated in
https://www.mycancergenome.org/content/gene/sf3b1/; Obeng et al (2019, Cancer Discovery, 9, 1493-1510; Rahman et al. (2020, Cell, 180,208-208.e1;
https://cancer.sanger.ac.uk/cosmic/gene/analysis?In=SF3B1; Seiler et al. (2018, Cell Rep, 23, 282-296.e4)

Kit and Uses Thereof

The present invention relates to a kit comprising a detection mean specific to the STIM1_{Ins} isoform, in particular any particular mean as disclosed above. Optionally, the kit may further comprise a leaflet.

It further relates to the use of such a kit for detecting the STIM1_{Ins} isoform, for detecting a disease as disclosed above or a susceptibility to develop the disease in a subject, especially a cancer or a myelodysplastic syndrome, for providing information on a prognosis of a subject having a disease as disclosed above, especially a cancer or a myelodysplastic syndrome, and for assessing a response or lack of response to a treatment in a subject or for providing information useful for assessing a response or lack of response to a treatment in a subject.

Further aspects and advantages of the present invention will be described in the following examples, which should be regarded as illustrative and not limiting.

### EXAMPLES

### Example 1

Results

Cancer-Associated Mutations of SF3B1 Lead to the Production of a Novel Splice Variant of STIM1, Named STIM1ᵢₙₛ.

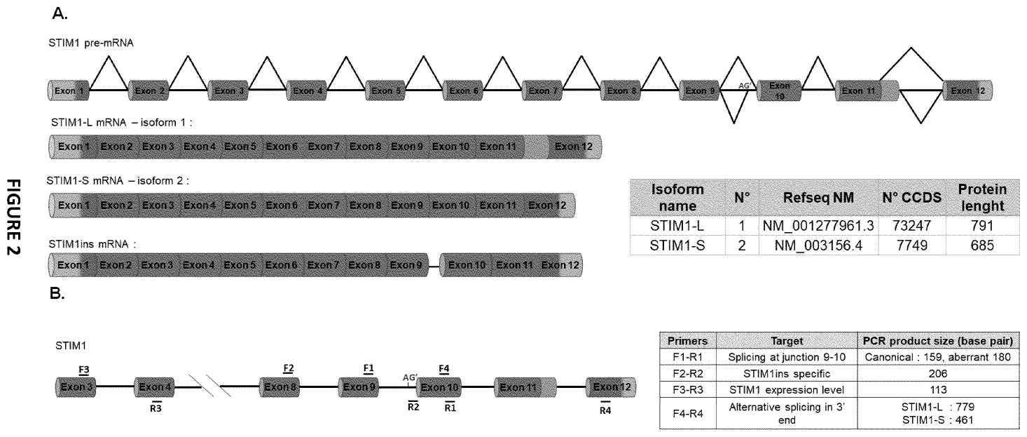

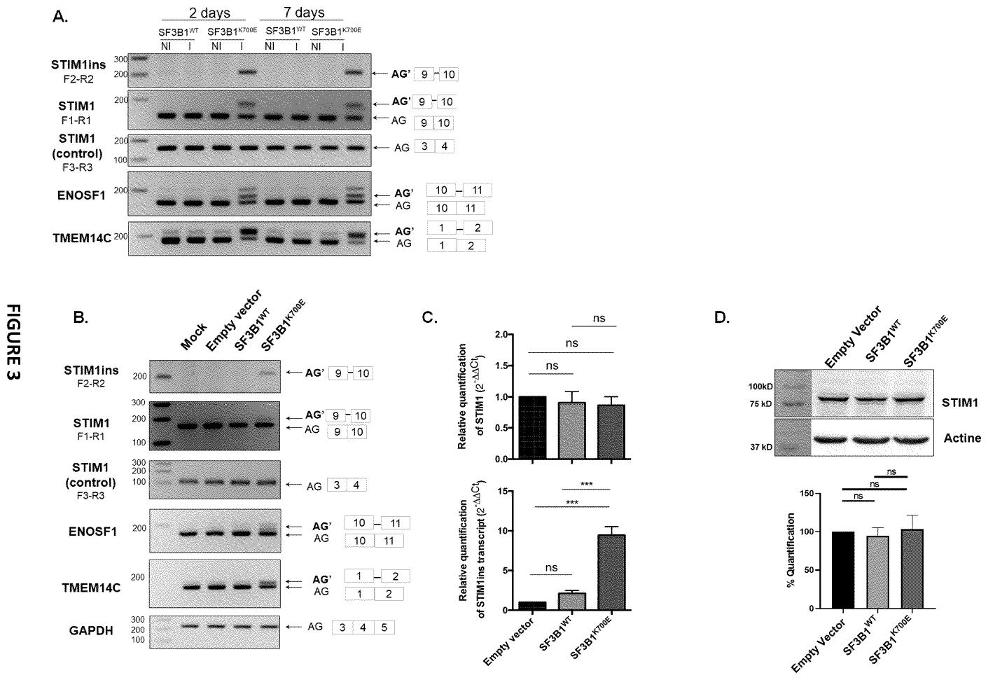

Among the multiple genes known to be differentially spliced in cells expressing SF3B1-mutated versus SF3B1^{WT} the inventors decided to focus on those involved in [Ca²⁺]i influx, more precisely on members of STIM1, ORAI and TRPC families. By crossing published RNAseq data generated in SF3B1-mutated malignancies, they found a novel STIM1 splice variant that was detected in all studies, to which the inventors gave the name of STIM1ᵢₙₛ. STIM1, as well as its paralogue STIM2, contains multiple exons, with a highly conserved exon structure, the main splice variant being STIM1S (short), which encodes a 685 amino acids long isoform. STIM1S is commonly named STIM1. A longer isoform, STIM1L, which is essentially expressed in muscles, results from an alternative splicing in which exon 11 is extended (FIG. 2), leading to the addition of 106 residues (aa 515-620) into the cytosolic actin-binding domain of STIM1. STIM1ᵢₙₛ splice variant retains 21 nucleotides from intron 9 into exon 10 (FIG. 1A), leading to the insertion of 7 amino acid residues into the coiled-coiled (CC) 3 segment of STIM-Orai-activating region (SOAR) domain of STIM1, in which lysine in position 413 was replaced by the NSLSSVFR sequence (FIG. 1B). Remarkably, this finding was very similar to what has been previously described for the STIM2.1 splice variant (STIM20). Indeed, STIM2.1 differs from the main STIM2.2 by the insertion of a cassette exon between exon 8 and 9 of STIM2.2, resulting in the insertion of eight highly conserved amino acid residues (VAASYLIQ) into the CC2 segment of SOAR domain of STIM2 (FIG. 1B). SOAR domain is a SOCE activation domain involved in the activation of ORAI1 and to a lesser extent to TRPC1. Thereby, both STIM1ᵢₙₛ and STIM2.2 isoforms contain a short amino acid sequence in the middle of SOAR domain, yet in a distinct CC segment.

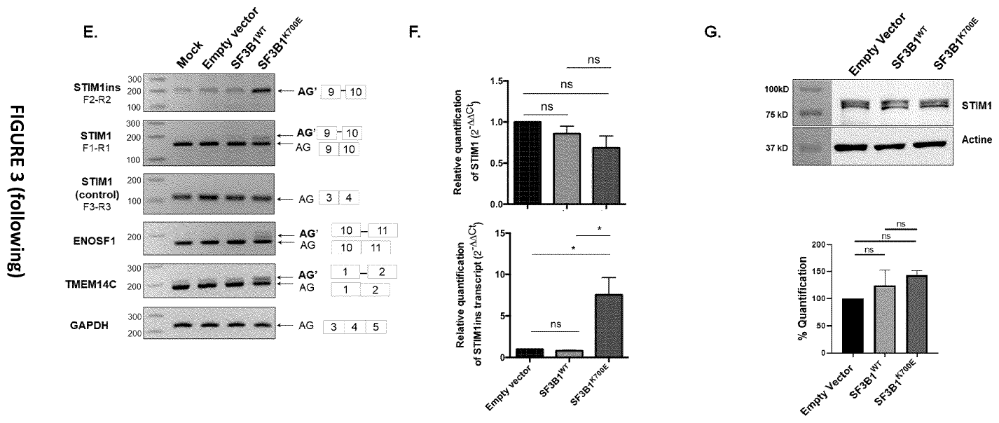

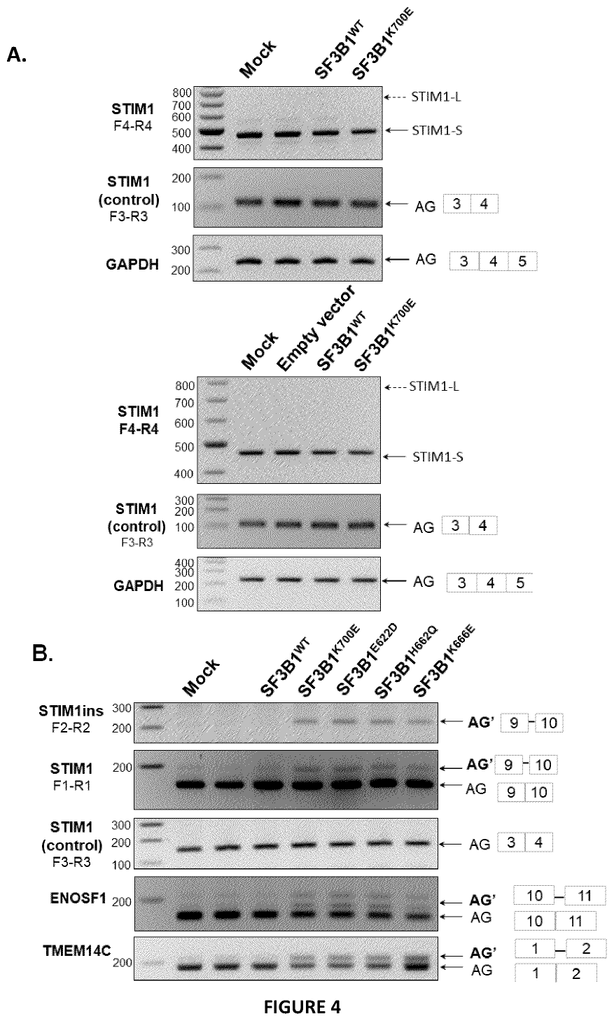

The inventors analysed all publicly available RNAseq data and confirmed that the STIM1ᵢₙₛ splice variant was significantly detected in all SF3B1-mutated pathologies, including breast cancer, uveal melanoma, CLL and MDS, with a Percent Splice-In (PSI) varying from 16 to 33% (FIG. 1C). To confirm this finding, the inventors quantified the level of STIM1ᵢₙₛ splice variant in mononuclear cells from MDS-RS patients harbouring SF3B1 mutations, in comparison to cells expressing wildtype SF3B1 (FIGS. 1D-1E). As expected, STIM1ᵢₙₛ was systematically and exclusively detected in SF3B1mut MDS-RS patients, with a very limited background signal in SF3B1^{WT} samples. To confirm that production of STIM1ᵢₙₛ transcripts was driven by SF3B1 hot spot mutations, the inventors generated human myeloid leukemic cell lines (K562) expressing wild type SF3B1 or the most frequent variant SF3B1K700E in MDS, under the control of a doxycycline inducible promoter. STIMᵢₙₛ transcript was exclusively produced upon expression of mutant SF3B1 (FIG. 3A). Moreover, transient expression of SF3B1^{K700E} in two independent cell lines, K562 and HEK293T, drove the formation of STIM1ᵢₙₛ transcript, as shown by classical and quantitative RT-PCR (FIG. 3B-C-E-F), without impacting total Stim1 transcript and protein levels (FIG. 3C-D-F-G). STIM1ᵢₙₛ transcript is most likely generated from STIM1S, and not STIM1L, as the latter could not be detected using primers allowing amplification of both alternate transcripts (FIG. 4A), which is expected considering that STIM1L is mostly expressed in muscle fibres. STIM1ᵢₙₛ was also produced upon expression of other frequent cancer-related SF3B1 variants, including K666E, E622D and H662Q (FIG. 4B).

Expression of SF3B1^{K700E} or of STIMᵢₙₛ does not Impact Store-Operated Ca²⁺ Entry

Eukaryotic cells have a number of mechanisms to induce Ca²⁺ entry but store-operated Ca²⁺ entry (SOCE) is an ubiquitous mechanism considered as the main Ca²⁺ entry pathway in non-excitable cells. SOCE is activated by a reduction in intracellular Ca²⁺ stores, mainly the ER ([Ca²⁺]ER), that evokes the opening of plasma membrane (PM) calcium channels leading to a Ca²⁺ influx. The two main families of proteins supporting SOCE are STIM (Stromal Interaction Molecule), comprising STIM1 and STIM2 serving as ER Ca²⁺ sensors and plasma membrane ORAI and TRPC calcium channels allowing Ca²⁺ entry from the extracellular medium. Store-dependent, STIM1-modulated, channels include the archetypal Ca²⁺ release-activated Ca2+ channels, comprised of ORAI1 subunits, as well as store-operated Ca²⁺ (SOC) channels involving ORAI1 but also and members of members of the TRPC proteins such as TRPC1.

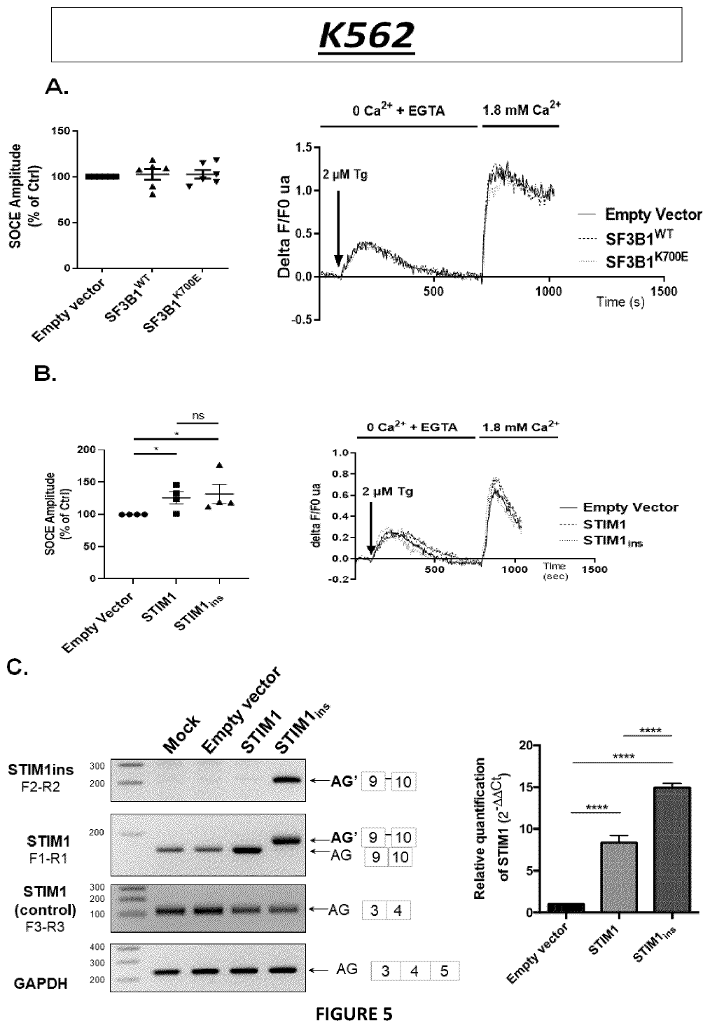

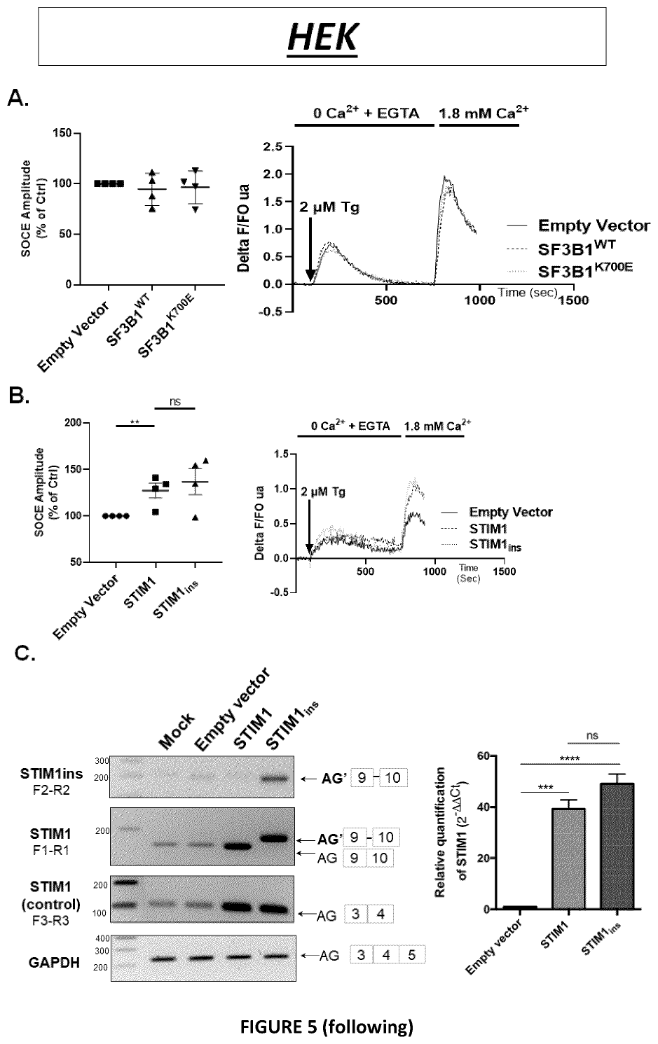

Alternative splicing of STIM1 and STIM2 genes produce distinct protein isoforms that differently regulate Ca²⁺ entry. For instance, STIM1L is able to activate SOCE more quickly than STIM1, a propriety compatible with a contractile activity found in skeletal muscle fibre cells, and is able to bind Orai1 more strongly than STIM1. As mentioned above, two splice variants of STIM2, STIM2.2 and STIM2.1, have been described with opposite function regarding SOCE activation. Remarkably, STIM1ᵢₙₛ isoform share structural similarities with STIM2.1 (STIM2β) in the sense that both contain an additional short amino acid sequence in the SOAR domain, yet in a distinct CC segment, in comparison to STIM1 and STIM2.1 respectively. Thereby, the inventors decided to investigate how STIM1, could regulate SOCE. Because STIM1ᵢₙₛ is specifically produced when SF3B1 is mutated, the inventors also evaluated the impact of SF3B1 mutations on SOCE. Transient expression of wild type SF3B1 or mutated SF3B1^{K700E} in K562 and HEK293T cells did not modify the release of Ca²⁺ stores, nor the amplitude of SOCE (FIG. 5A). The inventors next investigated the direct effect of STIM1ᵢₙₛ expression on SOCE. They inserted the sequence encoding NSLSSVFR at the right position (K413) into the cDNA of STIM1 to obtain a STIM1ᵢₙₛ construct, which also had V5 and FLAG tags in N-terminus and C-terminus, respectively (like STIM1 construct). Both STIM1 and STIM1ᵢₙₛ expressing plasmids were transiently introduced in K562 and HEK293T cells. By using quantitative RT-PCR, the inventors found that STIM1 and STIM1m were overexpressed compared to the empty vector condition, but with a slightly but non-significant higher expression level when STIM1ᵢₙₛ was expressed in comparison to STIM1, in both K562 (left) and HEK293 cells (right) (FIG. 5B). As expected, overexpression of STIM1 led to an increased amplitude of SOCE, but no significant modulatory effect was observed in cells expressing STIM1ᵢₙₛ compared to STIM1 (FIG. 5C). To ensure that partners of STIM1 were not limiting in the experimental conditions, the inventors co-transfected STIM1 or STIM1ᵢₙₛ together with Orai1 or TRPC1 in HEK293T cells. Here again, the expression of STIM1, and STIM1 led to the same results, confirming that STIM1l, does not activate SOCE stronger or lesser than STIM1. Other transfections using a different vector (STIM1ᵢₙₛ BTX) showed the same results.

Moreover, Western-blot analysis with antibodies raised again Nter STIM1, V5 tag or FLAG tag revealed a change of STIM1 glycosylation pattern when STIM1, was expressed in K562 or HEK293T cells, with the detection of two bands. STIM1 is known to be glycosylated at residues N131 and N171, which are located on either side of SAM domain, and substituting these two asparagines into glutamines leads to a conformational change of STIM1 and to its reduced presence at the plasma membrane. In the present study, although the glycosylation pattern of STIM1ᵢₙₛ was modified, no change in SOCE was observed.

In conclusion, despite structural similarities between STIM1ᵢₙₛ and STIM2.1, which negatively regulate SOCE, STIM1ᵢₙₛ appears to behave as the canonical STIM1 isoform regarding SOCE activation.

Expression of SF3B1^{K700E} Leads to an Increase of Plasma Membrane STIM1 Pool (STIM1_{PM}) and to an Enhancement of Constitutive Calcium Entry

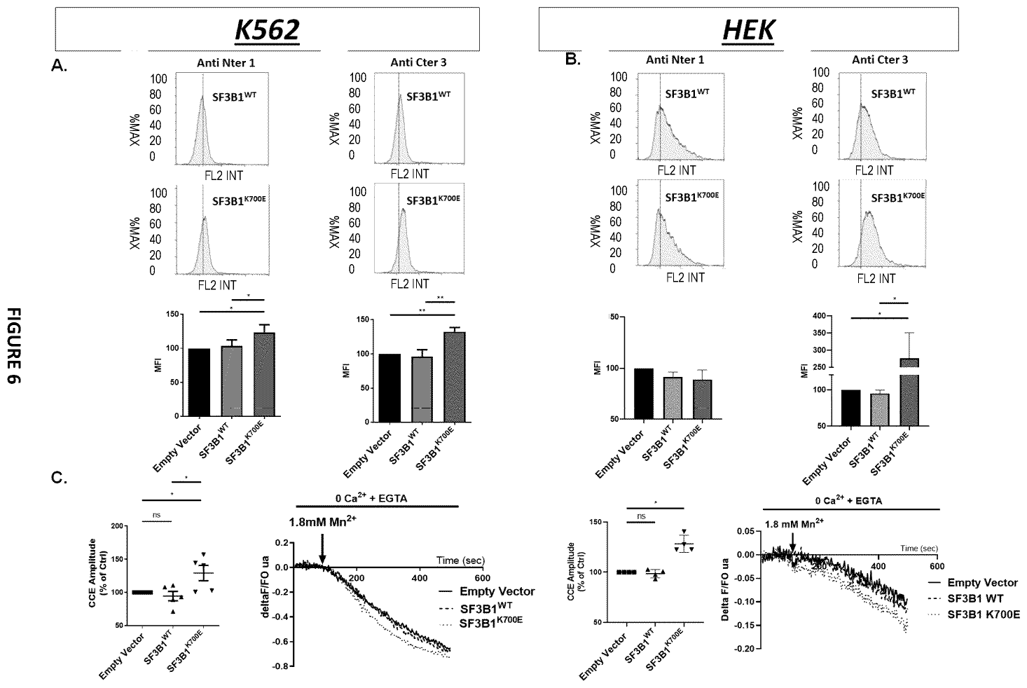

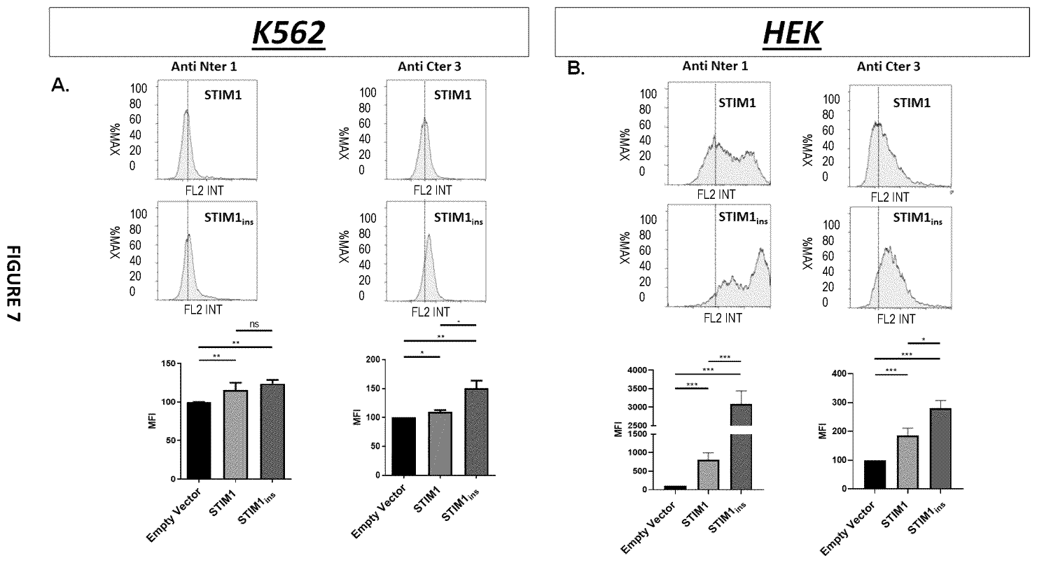

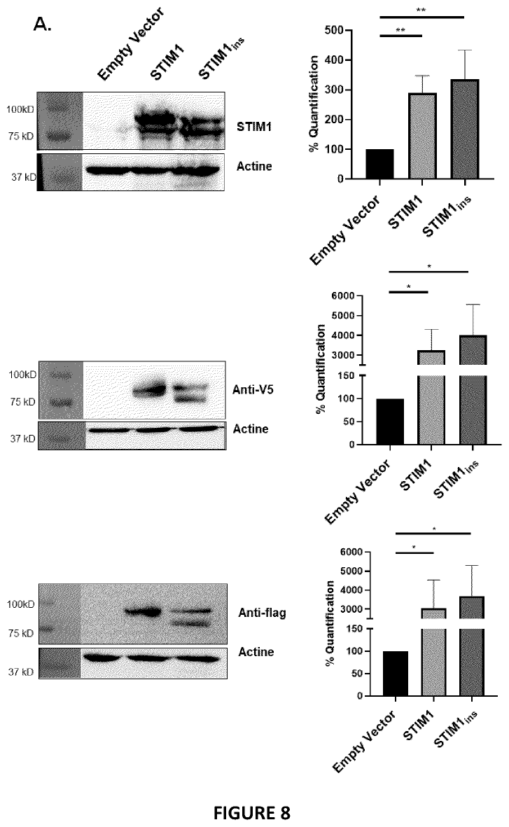

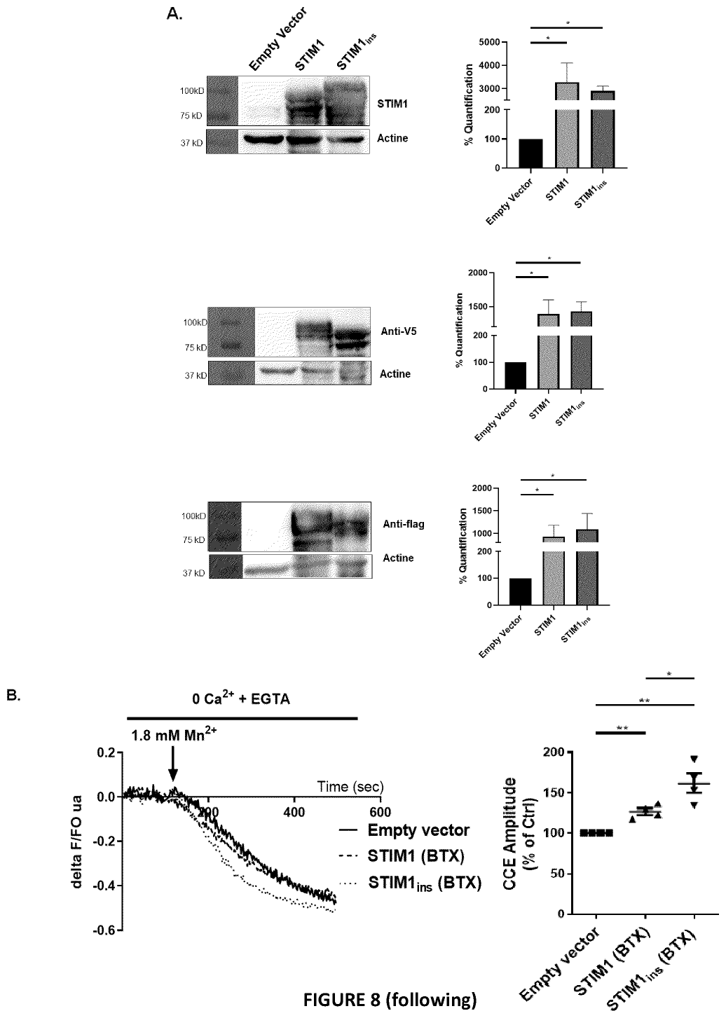

Changes in glycosylation patterns have been reported to cause localisation failures, including a decreased rate of the STIM1 pool localized at the plasma membrane (STIM1_{PM}). STIM1_{PM} has been described for twenty years with a single topology, consisting of an extra-cellular N-terminal domain and a cytosolic C-terminal domain. Recently, the inventors reported a dual topology of STIM1, with the N-terminal domain (N-terₒᵤₜ) or the C-terminal domain (C-terₒᵤₜ) being able to face the extracellular medium. Notably, STIM1 glycosylation reduction leads to a decrease of STIM1_{PM} N-terₒᵤₜ and to an increase of STIM1_{PM} C-terₒᵤₜ, as the inventors showed in Panc1_{WT} cell line transfected with STIM1 glycosylation mutants or in cells treated with tunicamycin. Using the same strategy, the inventors investigated the level and the orientation of STIM1_{PM} in cells transiently expressing SF3B1^{WT} or SF3B1^{K700E}. The level of STIM1 N-Terₒᵤₜ was slightly increased in K562 cells expressing SF3B1^{K700E} compared to SF3B1^{WT} condition (FIG. 6A), but was unmodified in HEK293T cells (FIG. 6B). Remarkably, in both cell lines expressing SF3B1^{K700E}, the level of STIM1 C-terₒᵤₜ was increased (FIGS. 6A and 6B), with a 3-fold change in HEK293T cell line. Therefore, expression of SF3B1^{K700E} in both cell lines leads to an increased level of plasma membrane STIM1 pool, mostly in C-terₒᵤₜ topology consistent with the reduction of STIM1 glycosylation observed in cells expressing STIM1ᵢₙₛ. (FIG. 8A).

It has been reported that STIM1_{PM} does not regulate SOCE, but has been implicated in Ca²⁺ arachidonate-regulated Ca²⁺-selective channels, as well as in another calcium influx, named calcium constitutive entry (CCE), which has been recently described in B lymphocyte cells. CCE is observed in absence of cell stimulation and is supported by Orai1 and TRPC1, and is regulated by STIM1_{PM} oriented Nter out or Cter out. This entry is enhanced in cancer cells such as CLL cells along with an increased in STIM1_{PM}. So, the inventors next measured CCE in cells expressing SF3B1^{WT} or SF3B1^{K700E} in K562 and HEK293T cell lines. CCE was increased upon expression of SF3B1^{K700E} in both cell lines (FIG. 6C), probably due to increased STIM1 C-terot level.

Expression of STIM1ᵢₙₛ Leads to an Enhancement of Constitutive Calcium Entry

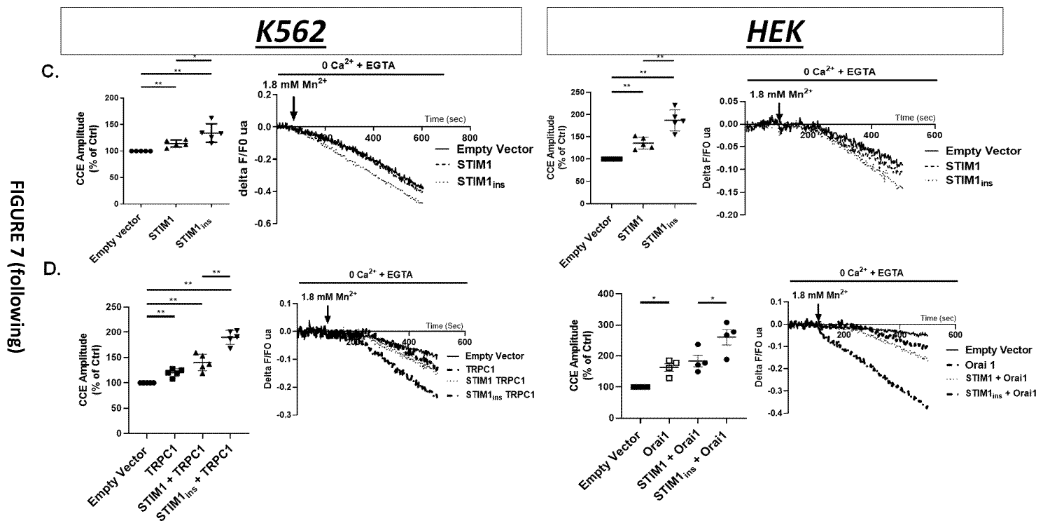

Considering the effect of SF3B1^{K700E} expression on the topology of STIM1_{PM} and on CCE, the inventors next decided to measure STIM1_{PM} levels and CCE in cells transiently expressing STIM1ᵢₙₛ. As mentioned, the level of STIM1_{PM}, especially STIM1 C-terₒᵤₜ, was increased in both K562 and HEK293T cells expressing STIM1ᵢₙₛ compared to STIM1 (FIGS. 7A and B). An increase of STIM1 N-terₒᵤₜ was observed only in HEK293T cells (FIG. 7B), with a 4-fold change. Moreover, the inventors found that CCE was increased in cells overexpressing STIM1ᵢₙₛ, compared to cells overexpressing STIM1, in both cell types (FIG. 7C). Similar results were obtained when we used another STIM1ᵢₙₛ vector (STIM1ᵢₙₛ BTX) in K562 cells (FIG. 8B). To confirm the effect of STIM1ᵢₙₛ on CCE, the inventors expressed STIM1 or STIM1ᵢₙₛ in HEK293T cells, together with TRPC1 or Orai1, which are calcium channels supporting ECC in B lymphocyte cells and other cell types. When TRPC1 or Orai1 was over-expressed, CCE was increased, confirming the implication of these channels in CCE. Co-expression of STIM1ᵢₙₛ with TRPC1 or Orai1 led a higher increase of CCE compared to co-expression conditions with canonical STIM1 (FIG. 7D). Taken together, these data confirm the impact of STIM1ᵢₙₛ on the topology of STIM1_{PM} and on CCE, suggesting an over-activation effect of Orai1 and TRPC1 channels by the pool of STIM1ᵢₙₛ located at the plasma membrane. Since the production of STIM1ᵢₙₛ mimics the effect of SF3B1^{K700E} expression on CCE, it is likely that the increased CCE observed in SF3B1^{K700E} cells is mediated by STIM1ᵢₙₛ expression.

Increase of CCE is Due to an Enhancement in STIM1ᵢₙₛ Localized at the Plasma Membrane, as Shown by the Block of Increase of CCE by Anti-STIM1 Antibodies.

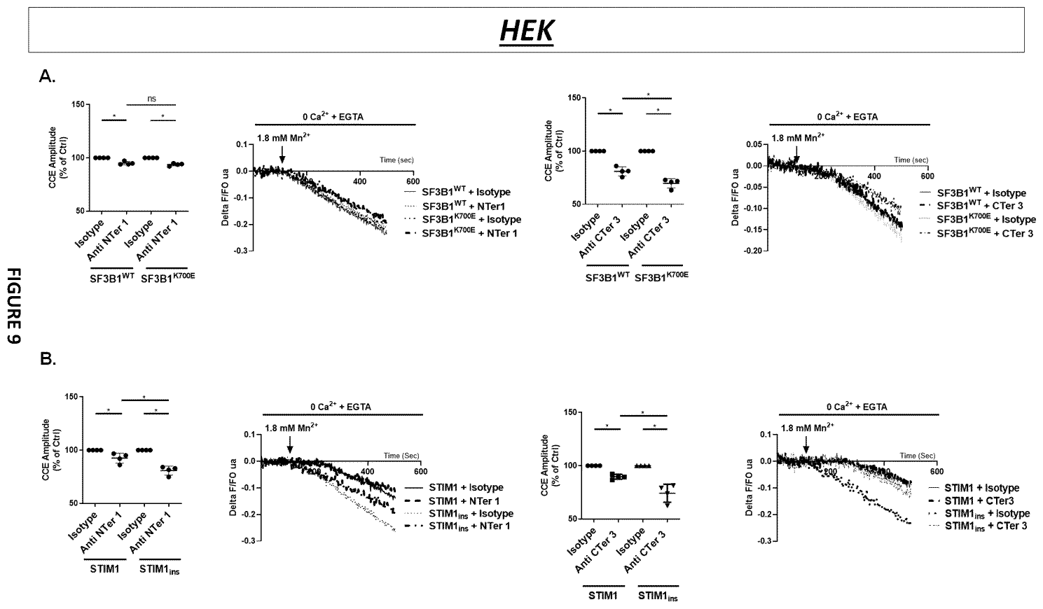

To find out whether the increase of CCE observed in cells expressing SF3B1^{K700E} or STIM1ᵢₙₛ was mediated by the pool of STIM1ᵢₙₛ at the plasma membrane, the inventors decided to block STIM1_{PM} with monoclonal anti-STIM1 antibodies in HEK293T cells expressing SF3B1^{K700E} or STIM1ᵢₙₛ, and to measure CCE. Firstly, no effect of anti-STIM1 antibody treatment was observed on SOCE confirming that these antibodies targeted only STIM1_{PM} and not STIM1_{RE} (FIGS. 8A and B) and that SOCE activation is independent of STIM1_{PM}. Secondly, the inventors show an increased inhibition of CCE when using antibodies targeting the C-terminal domain of STIM1 in cells expressing SF3B1^{K700E} compared to SF3B1^{WT}, and no enhancement of inhibition when using a N-terminal antibody (FIG. 9A). This observation is consistent with the fact that an increase of STIM1 C-terₒᵤₜ, and not N-terₒᵤₜ, was observed in cells expressing SF3B1^{K700E} (FIG. 6A) and confirm the regulation of CCE by STIM1_{PM} C-terₒᵤₜ. Finally, blocking STIM1_{PM} by antibodies targeting either the N or the C-terminal domain of STIM1 in cells overexpressing STIM1 or STIM1ᵢₙₛ, led to an inhibition of CCE, which was greater in STIM1ᵢₙₛ conditions (FIG. 9B). Taking together these data confirm that the increase of CCE observed in expressing SF3B1^{K700E} or STIM1ᵢₙₛ is due to the pool of STIM1ᵢₙₛ located at the plasma membrane.

Discussion

The inventors identified a novel STIM1 transcript specifically produced in SF3B1 mutated cancers, which encodes a novel STIM1 isoform with an insertion of eight amino acids in the SOAR domain of STIM1. They showed that STIM1ᵢₙₛ is involved in the regulation of constitutively active Ca²⁺ entry, probably due to an increased fraction of STIM1.localized at the plasma membrane, which is mainly inserted with its C-terminus end facing the extracellular medium. Surprisingly, the insertion of eight amino acids in the SOAR domain of STIM1, which is involved in STIM1 dimerization and activation, does not induce any significant SOCE perturbations, in contrast to what has been observed for the other STIM variants described to date.

STIM1ᵢₙₛ does not modulate SOCE. Given the importance of the domain modified in STIM1ᵢₙₛ, the inventors were expecting profound changes of the conformational structure of STIM1 CC3 domain and in STIM1 homodimerization. Very surprisingly, the present results clearly demonstrate that STIM1 and STIM1ᵢₙₛ regulate SOCE in a similar way. This, in contrast to what has been described for STIM1L or STIM2.1, the newly discovered STIM1 isoform, STIM1ᵢₙₛ, does not modulate differently the activation of SOCE, compared to STIM1, despite a higher expression of STIM1ᵢₙₛ.

STIM1ᵢₙₛ at the plasma membrane regulates constitutive Ca²⁺ influx. Here the inventors show that expression of STIM1ᵢₙₛ induces a significant increase in CCE that could be reversed by a cell pre-treatment with antibodies targeting the C-terminus end of STIM1. Data from the present study suggest that STIM1ᵢₙₛ controls CCE and that an increase in STIM1ᵢₙₛ at the plasma membrane would enhance this Ca²⁺ entry in the context of mutated SF3B1 expression. Increase in CCE observed in cells over-expressing STIM1m reinforce the fact that the inventors identified a functional role for this new STIM1 isoform as a regulator of a calcium entry.

STIM1ᵢₙₛ as a new disease associated antigen. Based on the present observations, STIM1ᵢₙₛ is only observed in cells expressing mutated SF3B1 and its expression is therefore not associated to a specific cell type but rather to any cell type with somatic SF3B1 mutations. Primary tumors with SF3B1 mutations should display STIM1ᵢₙₛ expression, including among others CLL, uveal melanoma, breast and pancreatic cancer independently of the mutated amino acid. Therefore, STIM1ᵢₙₛ may represent an important tumor marker but also a specific tumor antigen.

Materials and Methods

Patients:

Bone marrow samples were obtained from patients followed up in Brest University Hospital and in St Brieuc Hospital, and collected in the Centre de Ressources Biologiques (CRB) of Brest Hospital, which is referenced by the BioBank structure under a unique identifier number BB-0033-00037. The CRB is certified according to the French norm NF S 96-900 “CRB management system and quality of biological resources”. Written informed consent was obtained from all subjects, and the experiments conformed to the principles set out in the Declaration of Helsinki. Samples from MDS patients were the same used in a previous study (Bergot et al., 2020).

Cell Culture:

Human Embryo Kidney 293T (HEK 293T) and K562 cell line were obtained from American Type Culture Collection (ATCC). HEK 293T cells were cultured in Dubelcco's Minimal Essential Medium (DMEM 1X, Glutamax; Gibco) supplemented with 10% of Fetal Bovin Serum (FBS, Gibco) and 1% of Pennicilin/Streptomycin at 37° C. and 5% CO₂. K562 and K562-derived cell lines were cultured in RPMI 1640 medium (Gibco), supplemented with 10% FBS (Gibco) and 4 mM of L-glutamine (Gibco), at 37° C. and 5% of CO₂. Inducible stables cells lines were obtained as described in a previous study (Bergot et al., 2020) and SF3B1 expression was induced by treating cells with 2 μg/mL of doxycycline (Sigma).

Transient Transfection:

HEK 293T cells were transfected with 5 μg of an expression vector containing the STIM1 gene (pSTIM1 vector: V5-STIM1-Flag or V5-STIM1ᵢₙₛ-Flag), the Orai1 gene (Orai1 vector: Orai1-Flag), the TRPC1 gene (TRPC1 vector: TRPC1-Flag) or on optimized version of SF3B1ORF (SF3B1 vector: pCMV-SF3B1^{WT} or pCMV-SF3B1^{K700E}) or a corresponding empty vector, using the transfecting agent lipo293 (TebuBio) following the commercial recommendations in OptiMEM medium (Gibco). For co-transfections, cells were transfected with 2.5 μg of corresponding vector. K562 cells were transfected by electroporation using Cell Line Nucleofector™ Kit V (Amaxa, Lonza) according to the manufacturer's instructions. Transfections were done with 2×10⁶ cells and 4 μg of plasmid. All the analysis were realized 48 hours after transfection.

RNA Analysis:

RNA was extracted from bone marrow mononuclear cells of MDS patients using the Nucleospin RNA kit (Macherey-Nagel) or TRIzol (Invitrogen)-phenol/chloroform extraction. RNA was extracted from K562 and HEK293T cells using nucleospin RNA kit (Macherey-Nagel). RNA integrity was checked by agarose gel electrophoresis. Reverse transcription was performed with High Capacity cDNA reverse transcription kit (Applied Biosystems). PCR was performed with the GoTaq® G2 DNA Polymerase kit (Promega). Primers used are listed in Table 1. PCR products were analyzed on 2 or 3% agarose gels (Molecular Biology Grade Agarose, Eurobio). Quantitative real-time PCR was performed with Power SYBR™ Green PCR Master Mix (Applied Biosystems) with a StepOnePlus Real-Time PCR System. The 2^{nd} method was used to analyze the results using GAPDH as a reference gene.

TABLE 1

| | | | | |
|---|---|---|---|---|
| inverse | HD cloning | STIM1ins- | STIMins- | ttctctttcctctgtcttcagGCAAGCACTGAGCGA |
| | from | pCl- | F1 | GGTGAC (SEQ ID NO: 7) |
| | pCI-neo- | neo | | |
| | STIM1 | | | |
| PCR | | pCl-neo | reverse-R1 | TTAGCTGTTAGAATTTTATGATCTACATCATCC |
| | | | | (SEQ ID NO: 8) |
| PCR | STIM1 | splicing | forward-F1 | CACTCTTTGGCACCTTCCAC (SEQ ID NO: 9) |
| | | | reverse-R1 | GACAATCTGGAAGCCACAGAG (SEQ ID NO: 10) |
| | STIM1 ins | | forward-F2 | CTGACACATGAGGTGGAGGTG (SEQ ID NO: 11) |
| | | | reverse-R2b | GCCTGAAGACAGAGGAAAGAGAAC (SEQ ID NO: 12} |
| | STIM1 | control of | forward-F5 | GTGAAACACAGCACCTTCCA (SEQ ID NO: 13) |
| | | expression | | |
| | | | reverse-R5 | CACTGTACCACCTCATCCACG (SEQ ID NO: 14) |
| | | splicing | forward-F4 | GACGACGTGGATGACATGGA (SEQ ID NO: 15) |
| | | | reverse-R4 | GATGGTGTGTCTGGGTCTGG (SEQ ID NO: 16) |
| | ORAI2 | splicing | forward-F1 | GGGCGAGGGCAGCTC (SEQ ID NO: 17) |
| | | | reverse-R1 | AGGAGCAGAGGGGTCGATAG (SEQ ID NO: 18) |
| | | control of | forward-F2 | CACCTCTAACCACCACTCGG (SEQ ID NO: 19) |
| | | expression | | |
| | | | reverse-R2 | CGGGTACTGGTACTGCGTC (SEQ ID NO: 20) |
| | ORAI1 | control of | forward-F1 | CGCCAAGCTTAAAGCCTCCA (SEQ ID NO: 21) |
| | | expression | | |
| | | | reverse-R1 | CTGATCATGAGCGCAAACAGG (SEQ ID NO: 22) |
| | TMEM14C* | splicing | forward | GACACCTCGCAGTCATTCCT (SEQ ID NO: 23) |
| | | | reverse | TGATCCCACCAGAAGCAACC (SEQ ID NO: 24) |
| | ENOSF1* | | forward | GGGTGCTGATCTCCAGGATG (SEQ ID NO: 25) |
| | | | reverse | GGAGGTTGGCTCCTCAATCC (SEQ ID NO: 26) |
| | GAPDH | control of | forward | GAGTCAACGGATTTGGTCGT (SEQ ID NO: 27) |
| | | expression | | |
| | | | reverse | TTGATTTTGGAGGGATCTCG (SEQ ID NO: 28) |

Western Blot:

Protein extraction was performed on K562 cells on ice with a lysis buffer containing: 20 mM Tris HCl pH 7.5, 150 mM NaCl, 1 mM EDTA, 1 mM EGTA, 1% Triton X100, 2.5 mM Na+ pyrosodium tetraphosphate, 1 mM glycerophosphate, 1 mM Na+ orthovanadate, 1 μg/ml leupeptin and a protease inhibitor cocktail (Sigma-Aldrich, P8340). Protein extracts were sonicated and centrifuged for 20 min at 15,000 g. Protein concentration of cell lysates were determined using the Folin method. 75 μg of proteins were run on SDS-PAGE 10% polyacrylamide gels in denaturing conditions, and then transferred into PVDF (PolyVinyliDene Fluoride) membrane sheets. Unspecific blocking was done by incubation with 5% fat milk in PBS, 0.1% tween 20 for 1 hour at room temperature. Blots were incubated overnight at 4° C. with 5% fat milk in PBS, 0.1% tween 20, containing rabbit monoclonal anti-STIM1 (Anti STIM1 antibody, HPA012123 Sigma; 1:1,000 dilution) or mouse monoclonal anti-STIM1 (GOK BD; 1:1,000 dilution) or mouse monoclonal anti-V5 (V5 Tag Monoclonal antibody R96025 Invitrgen; 1:1,000 dilution) or mouse monoclonal anti-Flag (Monoclonal AntiFlag M2 antibody F3165 Sigma; 1:1,000 dilution) or mouse monoclonal anti-GAPDH antibody (6C5 clone Abcam; 1:10,000 dilution). Blots were incubated with Horseradish Peroxydase (HRP)-conjugated goat anti-mouse or anti-rabbit after washing with PBS, 0.1% tween 20 and revealed with the Luminata Forte reagent. All results were normalized upon GAPDH quantification.

For Quenching Measurement:

96 wells plate were pre-coated overnight at 37° C. with 30 μl of cell Tak solution (Corning 10317081). After one wash with distilled water, 5.10⁴ K562 cells were loaded for 1 hour at 37° C. with Fura-2 according to the manufacturer's protocol.

5.10⁴ HEK 293T cells were seeded overnight in 96 wells plate. Cells were loaded for 1 hour at 37° C. in with Fura-2 according to the manufacturer's protocol.

The Fura-2 QBT™ was aspirated and replaced by an equal volume of free Ca²⁺ Hepes-buffered solution containing (in mM): 135 NaCl, 5 KCl, 1 MgCl₂, 1 EGTA, 10 Hepes, 10 glucose, pH adjusted at 7.45 with NaOH. Intracellular calcium level variations were monitored by using the FlexStation 3™ (Molecular Devices, Berkshire, UK). Mn²⁺ quenching influx was measured as Fura-2 fluorescence quenching at excitation wavelength of 360 nm (isobestic wavelength) and at the emission wavelength of 510 nm.

For SOCE Measurement:

96 wells plate were pre-coated overnight at 37° C. with 30 μl of cell Tak solution (Corning 10317081). After one wash with distilled water, K562 cells were loaded for 1 hour at 37° C. in with Fura-2 according to the manufacturer's protocol.

5.10⁴ HEK 293T cells were seeded overnight in 96 wells plate. Cells were loaded 1 hour at 37° C. in with Fura—according to the manufacturer's protocol.

The Fura-2 QBT™ was aspirated and replaced by an equal volume of free Ca²⁺ Hepes-buffered solution containing (in mM): 135 NaCl, 5 KCl, 1 MgCl₂, 1 EGTA, 10 Hepes, 10 glucose, pH adjusted at 7.45 with NaOH. Intracellular calcium level variations were monitored by using the FlexStation 3™ (Molecular Devices, Berkshire, UK), Dual excitation wavelength capability permits ratiometric measurements of Fura-2AM peak emissions (510 nm) after excitations at 340 nm and 380 nm. Modifications in the 340/380 ratio reflect changes in intracellular-free Ca²⁺ concentrations. The SOCE was elicited by releasing the Ca²⁺ stores from the endoplasmic reticulum with thapsigargin (2 μM) solution under Ca²⁺-free conditions to determine the magnitude of intracellular Ca²⁺ release (Hepes-buffered solution). Next, cells were returned to a Ca²⁺-containing Hepes-buffered solution to measure SOCE. The magnitude and speed of SOCE were estimated.

Flow Cytometry Detection of STIM1_{PM}.

For determination of the amount of STIM1 using flow cytometry, 5×10⁶ cells (HEK or K562) were used per condition. Cells were centrifuged for 5 min at 300 g and incubated with 50 μL of PBS containing anti-STIM1 antibody directed against the N terminus (1 μl GOK-PE, BD Biosciences; 20 μg/ml) or the C terminus (5 μl CDN3H4 sc-66173, Santacruz; 10 μg/ml) or 0.5 μL of an isotype control (PE Mouse IgG2a, k isotype, Biolegend 20 μg/ml) for 20 min on ice. After 3 washes in PBS, the determination of the mean fluorescence intensity (MFI) of STIM1_{PM} required a minimum of 5000 events. The results were standardized to those obtained with isotype controls. Data were analysed using Kaluza 2.1 software (Beckman-Coulter).

### Example 2

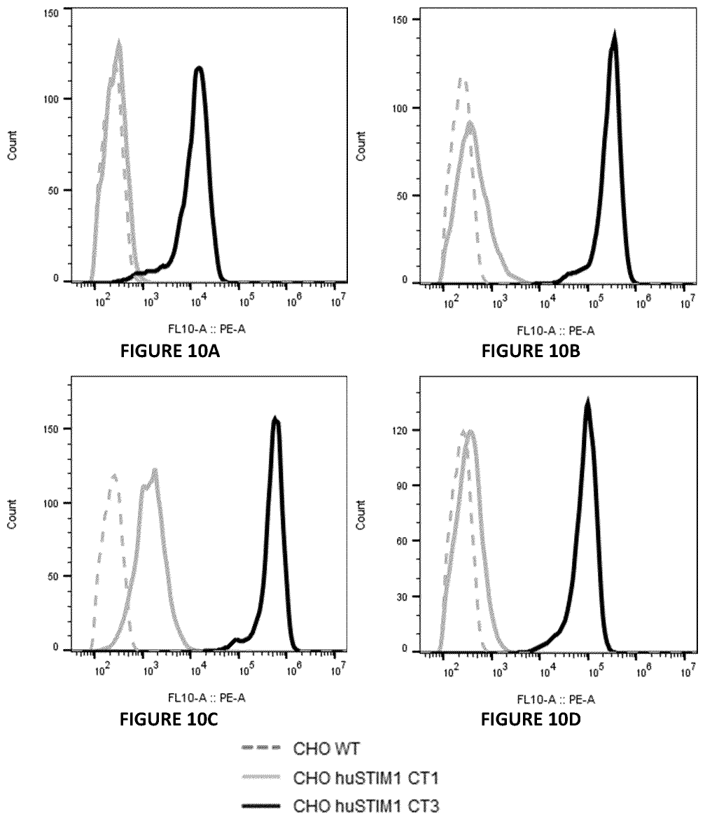

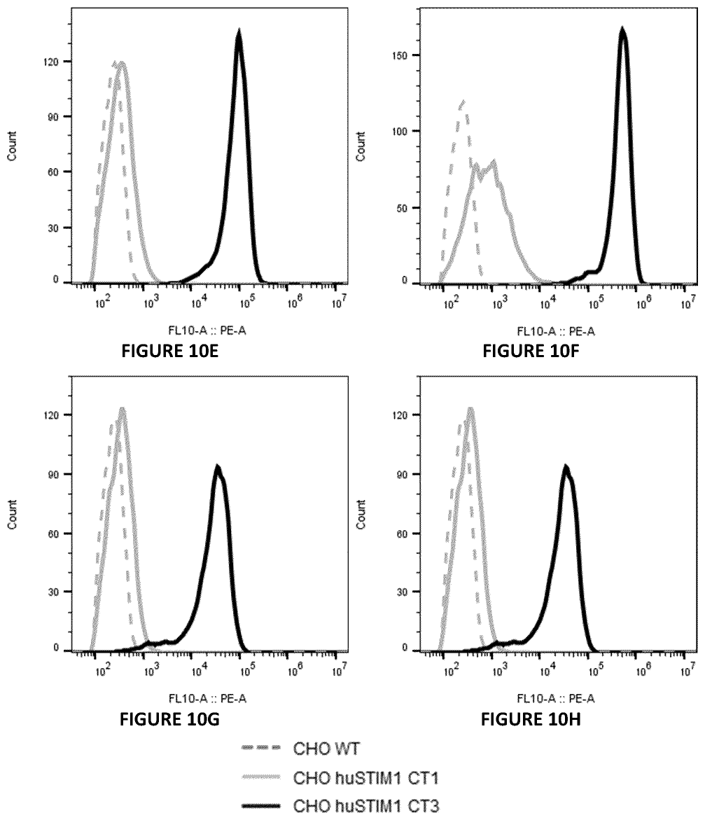

Results Antibodies were generated by mice immunization with a 12 amino acids-long peptide centered on the STIM1ins sequence (SEQ ID NO: 32: CTANSLSSFRQA) coupled to BSA as a carrier. 37 antibodies have been tested in the secondary screening of hybridomas supernatants. 8 antibodies have been identified as strong and selective binders of the STIM_{Ins} isoform as shown in FIG. 10, namely 1E05, 1F02, 2F12, 4B05, 8E11, 15G09, 15H01 and 16A08.

Materials and Methods

1_Immunization protocol: Five 8 weeks-old BALB/c female mice were immunized using a 12 amino acids-long peptide centered on the STIM1Ins sequence (in bold) (SEQ ID NO: 32: CTANSLSSFRQA), coupled to BSA as a carrier. Immunization was performed by 3 IP injections of 50 μg of peptide in emulsion with complete (1^{st} IP) or incomplete (2^{nd} and 3^{rd} IPs) Freund adjuvant, followed by 1 last intravenous boost with 15 μg of peptide in PBS.

2_Serum titration by flow cytometry: Human STIM Cter transfectants were generated in CHO cells to express C-ter domain at the cell surface. Constructs consists of the intracellular and transmembrane domains of CD72 type 2 protein fused to C-term domain of STIM-1 (huSTIM1 CT1 construct) or to C-term domain of STIM-1 “Ins” variant (huSTIM1 CT3 construct). CHO wild type (WT), CHO huSTIM1 CT1 or CHO huSTIM1 CT3 cells were stained with CellTrace Violet (CTV, Invitrogen, #C34557) according to manufacturer's instructions.
Post-IP2 titration was performed using a mix of 100,000 CTV-stained CHO huSTIM1 CT3 cells and 100,000 unstained CHO WT into each well.
Post-IP3 titration was performed using 60,000 CHO huSTIM1 CT3 cells (CTV high), 60,000 CHO huSTIM1 CT1 cells (CTV low) and 60,000 unstained CHO WT cells mixed into each well.

Cells were washed 3 times in FACS buffer (PBS 1×, BSA 0.5%, EDTA 2 mM) before incubation with sera from Non-Immunized mice (NI) or sera sampled after IP injection, for 1h at +4° C., using anti-STIM Cter CDN3H4 clone diluted at 15 μg/mL in FACS buffer as a positive control. Cells were then washed 3 times prior to mouse antibodies revelation by incubation with Goat anti-Mouse IgG Fc specific-AlexaFluor 647 (Jackson ImmunoResearch, #115-606-071) for 45 min at +4° C.

Cell events were acquired on Attune NXT (Thermofisher) and data analysed using the FlowJo software (v10.8.0; Becton Dickinson).

Mice #11 and #13 showed best specific titers for CHO huSTIM1 CT3 cells. Splenocytes from these two mice were isolated and used for a standard fusion and hybridoma generation.

3_Primary and secondary screening of hybridoma supernatants by flow cytometry: Supernatants were harvested and incubated with 150 000 CHO huSTIM1 CT1 or CHO huSTIM1 CT3 transfectant cells per combination for 1 hour at 4° C. Cells were washed twice using FACS buffer before incubation with Goat anti human IgG-Fc-PE (1/200, Jackson ImmunoResearch #109-116-170) secondary antibody for 30 min at 4° C. After 2 additional washes, cells were resuspended in FACS buffer containing SYTOX® Blue Dead Cell Stain. Plates were analysed on a CytoFLEX cytometer (Beckman Coulter) and data processed using FlowJo software (BD Biosciences).

5_Hybridoma sequencing & combination testing: After RNA extraction, cDNAs were generated by reverse transcription and used as templates for PCR amplification of VH and VL domains with CloneAmp HiFi DNA Polymerase (Takara) using a designed pool of forward and reverse primers representative of all known Mus musculus immunoglobulin variable germline genes/alleles. PCR products were then directly cloned into heavy and light chains expression vectors before sequencing. For each hybridoma, a minimum of 8 heavy chain and 8 light chain clones were sequenced. Sequences were then analysed using IMGT web resources (httt://www.imgt.org).

6_Combination testing: If multiple heavy and/or light chains sequences were retrieved from a single hybridoma, additional PCR clones were sequenced to evaluate the frequency of each individual sequence from this polyclonal hybridoma and a “combination test” was performed to identify functional heavy/light chains combinations as follows. Unique VH/VL combinations were transiently expressed in HEK293 cells by lipofectamine transfection of the corresponding vectors. Supernatants were harvested and incubated with 150 000 CHO huSTIM1 CT1 or CHO huSTIM1 CT3 CHO transfectant cells per combination for 1 hour at 4° C. Cells were washed twice using FACS buffer before incubation with Goat anti human IgG-Fc-PE (1/200, Jackson ImmunoResearch #109-116-170) secondary antibody for 30 min at 4° C. After 2 additional washes, cells were resuspended in FACS buffer containing SYTOX® Blue Dead Cell Stain. Plates were analysed on a CytoFLEX cytometer (Beckman Coulter) and data processed using FlowJo software (BD Biosciences).

SEQUENCE LISTING

| SEQ ID NO | Description |
|---|---|
| 1 | Isoform STIM1_{Ins} |
| 2 | Coding sequence of isoform STIM1_{Ins} |
| 3 | Amino acid sequence of isoform STIM1_{Ins} from position |
| | 411 to 420 |
| 4 | Nucleic acid sequence encoding the amino acid sequence |
| | of isoform STIM1_{Ins} from position 411 to 420 |
| 5 | Isoform STIM1S |
| 6 | Coding sequence of isoform STIM1S |

1-26. (canceled)

27. An antibody specific to an isoform of STIM1 comprising SEQ ID NO: 1, which differentially binds to said isoform in comparison to an isoform of STIM1 comprising SEQ ID NO: 5, or an antigen-binding fragment thereof.

28. The antibody or antigen-binding fragment thereof of claim 27, wherein the antibody comprises:
(i) a heavy chain variable domain comprising CDR 1, 2 and 3 of the heavy chain variable region of SEQ ID NO: 33 and (ii) a light chain variable domain comprising CDR 1, 2 and 3 of the light chain variable region of SEQ ID NO: 34;
(ii) a heavy chain variable domain comprising CDR 1, 2 and 3 of the heavy chain variable region of SEQ ID NO: 35 and (ii) a light chain variable domain comprising CDR 1, 2 and 3 of the light chain variable region of SEQ ID NO: 36;
(iii) a heavy chain variable domain comprising CDR 1, 2 and 3 of the heavy chain variable region of SEQ ID NO: 37 and (ii) a light chain variable domain comprising CDR 1, 2 and 3 of the light chain variable region of SEQ ID NO: 38;
(iv) a heavy chain variable domain comprising CDR 1, 2 and 3 of the heavy chain variable region of SEQ ID NO: 39 and (ii) a light chain variable domain comprising CDR 1, 2 and 3 of the light chain variable region of SEQ ID NO: 40;
(v) a heavy chain variable domain comprising CDR 1, 2 and 3 of the heavy chain variable region of SEQ ID NO: 41 and (ii) a light chain variable domain comprising CDR 1, 2 and 3 of the light chain variable region of SEQ ID NO: 42;
(vi) a heavy chain variable domain comprising CDR 1, 2 and 3 of the heavy chain variable region of SEQ ID NO: 43 and (ii) a light chain variable domain comprising CDR 1, 2 and 3 of the light chain variable region of SEQ ID NO: 44; or
(vii) a heavy chain variable domain comprising CDR 1, 2 and 3 of the heavy chain variable region of SEQ ID NO: 45 and (ii) a light chain variable domain comprising CDR 1, 2 and 3 of the light chain variable region of SEQ ID NO: 46; or
(viii) a heavy chain variable domain comprising CDR 1, 2 and 3 of the heavy chain variable region of SEQ ID NO: 47 and (ii) a light chain variable domain comprising CDR 1, 2 and 3 of the light chain variable region of SEQ ID NO: 48.

29. The antibody or antigen-binding fragment thereof of claim 27, wherein the antibody comprises:
(i) a light chain variable domain comprising a light chain CDR1 (LCDR1) comprising SEQ ID NO: 121; a light chain CDR2 (LCDR2) comprising SEQ ID NO: 122; and a light chain CDR3 (LCDR3) comprising SEQ ID NO: 123; and a heavy chain variable domain comprising a heavy chain CDR1 (HCDR1) comprising SEQ ID NO: 49; a heavy chain CDR2 (HCDR2) comprising SEQ ID NO: 50; and a heavy chain CDR3 (HCDR3) comprising SEQ ID NO: 51;
(ii) a light chain variable domain comprising a LCDR1 comprising SEQ ID NO: 130; a LCDR2 comprising SEQ ID NO: 131; and a LCDR3 comprising SEQ ID NO: 132; and a heavy chain variable domain comprising a HCDR1 comprising SEQ ID NO: 58; a HCDR2 comprising SEQ ID NO: 59; and a HCDR3 comprising SEQ ID NO: 60;
(iii) a light chain variable domain comprising a LCDR1 comprising SEQ ID NO: 139; a LCDR2 comprising SEQ ID NO: 140; and a LCDR3 comprising SEQ ID NO: 141; and a heavy chain variable domain comprising a HCDR1 comprising SEQ ID NO: 67; a HCDR2 comprising SEQ ID NO: 68; and a HCDR3 comprising SEQ ID NO: 69;
(iv) a light chain variable domain comprising a LCDR1 comprising SEQ ID NO: 148; a LCDR2 comprising SEQ ID NO: 149; and a LCDR3 comprising SEQ ID NO: 150; and a heavy chain variable domain comprising a HCDR1 comprising SEQ ID NO: 76; a HCDR2 comprising SEQ ID NO: 77; and a HCDR3 comprising SEQ ID NO: 78;
(v) a light chain variable domain comprising a LCDR1 comprising SEQ ID NO: 157; a LCDR2 comprising SEQ ID NO: 158; and a LCDR3 comprising SEQ ID NO: 159; and a heavy chain variable domain comprising a HCDR1 comprising SEQ ID NO: 85; a HCDR2 comprising SEQ ID NO: 86; and a HCDR3 comprising SEQ ID NO: 87;
(vi) a light chain variable domain comprising a LCDR1 comprising SEQ ID NO: 166; a LCDR2 comprising SEQ ID NO: 167; and a LCDR3 comprising SEQ ID NO: 168; and a heavy chain variable domain comprising a HCDR1 comprising SEQ ID NO: 94; a HCDR2 comprising SEQ ID NO: 95; and a HCDR3 comprising SEQ ID NO: 96;
(vii) a light chain variable domain comprising a LCDR1 comprising SEQ ID NO: 175; a LCDR2 comprising SEQ ID NO: 176; and a LCDR3 comprising SEQ ID NO: 177; and a heavy chain variable domain comprising a HCDR1 comprising SEQ ID NO: 103; a HCDR2 comprising SEQ ID NO: 104; and a HCDR3 comprising SEQ ID NO: 105; or
(viii) a light chain variable domain comprising a LCDR1 comprising SEQ ID NO: 184; a LCDR2 comprising SEQ ID NO: 185; and a LCDR3 comprising SEQ ID NO: 186; and a heavy chain variable domain comprising a HCDR1 comprising SEQ ID NO: 112; a HCDR2 comprising SEQ ID NO: 113; and a HCDR3 comprising SEQ ID NO: 114.

30. The antibody or antigen-binding fragment thereof of claim 27, wherein the antibody is a chimeric, humanized or human antibody.

31. The antibody or antigen-binding fragment thereof of claim 27, wherein the antibody competes with an antibody selected from the group of 1E05, 1F02, 2F12, 4B05, 8E11, 15G09, 15H01 and 16A08 for binding to a polypeptide comprising SEQ ID NO: 1 or a fragment thereof, said fragment comprising SEQ ID NO: 3.

32. An isolated peptide of less than 50 amino acids in length and comprising SEQ ID NO: 3.

33. An in vitro method for detecting an isoform of STIM1 of SEQ ID NO: 1 comprising contacting a sample with a detection means specific to the isoform of STIM1 of SEQ ID NO: 1 and detecting the presence of the isoform of STIM1 of SEQ ID NO: 1.

34. The method of claim 33, wherein the detection means is an antibody specific to an isoform of STIM1 comprising SEQ ID NO: 1, which differentially binds to said isoform in comparison to an isoform of STIM1 comprising SEQ ID NO: 5, or an antigen-binding fragment thereof or a probe or primer specific to a coding sequence for the isoform of STIM1 of SEQ ID NO: 1, said probe or primer comprising at least 10 consecutive nucleic acids of SEQ ID NO: 4.

35. An in vitro method for detecting a cancer or a myelodysplastic syndrome or a susceptibility to develop a cancer or a myelodysplastic syndrome in a subject, wherein the method comprises detecting an isoform of STIM1 of SEQ ID NO: 1 in a sample from said subject by a method according to claim 33, the presence of the isoform of STIM1 of SEQ ID NO: 1 being indicative of a cancer or a myelodysplastic syndrome or a susceptibility to develop a cancer or a myelodysplastic syndrome in said subject.

36. An in vitro method for providing information on a prognosis of a subject having a cancer or a myelodysplastic syndrome, wherein the method comprises detecting an isoform of STIM1 of SEQ ID NO: 1 in a sample from said subject by a method according to claim 33, the presence of the isoform of STIM1 of SEQ ID NO: 1 being indicative of the prognosis in said subject.

37. A multispecific or bispecific antibody comprising a heavy chain variable domain and a light chain variable domain as defined any one of items (i) to (viii) of claim 28.

38. A chimeric antigen receptor (CAR) comprising a heavy chain variable domain and a light chain variable domain as defined any one of items (i) to (viii) of claim 28.

39. A cell comprising a CAR of claim 38.

40. A pharmaceutical composition comprising a modulator specific to an isoform of STIM1 of SEQ ID NO: 1 or a molecule targeting specifically an isoform of STIM1 of SEQ ID NO: 1 optionally linked to a detectable label or a drug.

41. A method of treating a disease comprising administering a modulator specific to an isoform of STIM1 of SEQ ID NO: 1 or a molecule targeting specifically an isoform of STIM1 of SEQ ID NO: 1 to a subject in need of treatment.

42. The method of claim 41, wherein the disease is associated with a splicing defect or a mutation or alteration in a splice factor gene.

43. The method of claim 41, wherein the disease is a cancer or myelodysplastic syndrome.

44. The method of claim 41, wherein:
a) the modulator or molecule comprises an antibody specific to an isoform of STIM1 comprising SEQ ID NO: 1, which differentially binds to said isoform in comparison to an isoform of STIM1 comprising SEQ ID NO: 5, or an antigen-binding fragment thereof;
b) a peptide of less than 50 amino acids in length and comprising SEQ ID NO: 3;
c) an inhibitor reducing or blocking specifically the expression of the isoform of STIM1 of SEQ ID NO: 1;
d) a multispecific or bispecific antibody comprising:
(i) a heavy chain variable domain comprising CDR 1, 2 and 3 of the heavy chain variable region of SEQ ID NO: 33 and (ii) a light chain variable domain comprising CDR 1, 2 and 3 of the light chain variable region of SEQ ID NO: 34;
(ii) a heavy chain variable domain comprising CDR 1, 2 and 3 of the heavy chain variable region of SEQ ID NO: 35 and (ii) a light chain variable domain comprising CDR 1, 2 and 3 of the light chain variable region of SEQ ID NO: 36;
(iii) a heavy chain variable domain comprising CDR 1, 2 and 3 of the heavy chain variable region of SEQ ID NO: 37 and (ii) a light chain variable domain comprising CDR 1, 2 and 3 of the light chain variable region of SEQ ID NO: 38;
(iv) a heavy chain variable domain comprising CDR 1, 2 and 3 of the heavy chain variable region of SEQ ID NO: 39 and (ii) a light chain variable domain comprising CDR 1, 2 and 3 of the light chain variable region of SEQ ID NO: 40;
(v) a heavy chain variable domain comprising CDR 1, 2 and 3 of the heavy chain variable region of SEQ ID NO: 41 and (ii) a light chain variable domain comprising CDR 1, 2 and 3 of the light chain variable region of SEQ ID NO: 42;
(vi) a heavy chain variable domain comprising CDR 1, 2 and 3 of the heavy chain variable region of SEQ ID NO: 43 and (ii) a light chain variable domain comprising CDR 1, 2 and 3 of the light chain variable region of SEQ ID NO: 44; or
(vii) a heavy chain variable domain comprising CDR 1, 2 and 3 of the heavy chain variable region of SEQ ID NO: 45 and (ii) a light chain variable domain comprising CDR 1, 2 and 3 of the light chain variable region of SEQ ID NO: 46; or
(viii) a heavy chain variable domain comprising CDR 1, 2 and 3 of the heavy chain variable region of SEQ ID NO: 47 and (ii) a light chain variable domain comprising CDR 1, 2 and 3 of the light chain variable region of SEQ ID NO: 48;
e) a CAR comprising:
(i) a heavy chain variable domain comprising CDR 1, 2 and 3 of the heavy chain variable region of SEQ ID NO: 33 and (ii) a light chain variable domain comprising CDR 1, 2 and 3 of the light chain variable region of SEQ ID NO: 34;
(ii) a heavy chain variable domain comprising CDR 1, 2 and 3 of the heavy chain variable region of SEQ ID NO: 35 and (ii) a light chain variable domain comprising CDR 1, 2 and 3 of the light chain variable region of SEQ ID NO: 36;
(iii) a heavy chain variable domain comprising CDR 1, 2 and 3 of the heavy chain variable region of SEQ ID NO: 37 and (ii) a light chain variable domain comprising CDR 1, 2 and 3 of the light chain variable region of SEQ ID NO: 38;
(iv) a heavy chain variable domain comprising CDR 1, 2 and 3 of the heavy chain variable region of SEQ ID NO: 39 and (ii) a light chain variable domain comprising CDR 1, 2 and 3 of the light chain variable region of SEQ ID NO: 40;
(v) a heavy chain variable domain comprising CDR 1, 2 and 3 of the heavy chain variable region of SEQ ID NO: 41 and (ii) a light chain variable domain comprising CDR 1, 2 and 3 of the light chain variable region of SEQ ID NO: 42;
(vi) a heavy chain variable domain comprising CDR 1, 2 and 3 of the heavy chain variable region of SEQ ID NO: 43 and (ii) a light chain variable domain comprising CDR 1, 2 and 3 of the light chain variable region of SEQ ID NO: 44; or
(vii) a heavy chain variable domain comprising CDR 1, 2 and 3 of the heavy chain variable region of SEQ ID NO: 45 and (ii) a light chain variable domain comprising CDR 1, 2 and 3 of the light chain variable region of SEQ ID NO: 46; or
(viii) a heavy chain variable domain comprising CDR 1, 2 and 3 of the heavy chain variable region of SEQ ID NO: 47 and (ii) a light chain variable domain comprising CDR 1, 2 and 3 of the light chain variable region of SEQ ID NO: 48;
or
f) a cell comprising a CAR, said CAR comprising:
(i) a heavy chain variable domain comprising CDR 1, 2 and 3 of the heavy chain variable region of SEQ ID NO: 33 and (ii) a light chain variable domain comprising CDR 1, 2 and 3 of the light chain variable region of SEQ ID NO: 34;
(ii) a heavy chain variable domain comprising CDR 1, 2 and 3 of the heavy chain variable region of SEQ ID NO: 35 and (ii) a light chain variable domain comprising CDR 1, 2 and 3 of the light chain variable region of SEQ ID NO: 36;
(iii) a heavy chain variable domain comprising CDR 1, 2 and 3 of the heavy chain variable region of SEQ ID NO: 37 and (ii) a light chain variable domain comprising CDR 1, 2 and 3 of the light chain variable region of SEQ ID NO: 38;
(iv) a heavy chain variable domain comprising CDR 1, 2 and 3 of the heavy chain variable region of SEQ ID NO: 39 and (ii) a light chain variable domain comprising CDR 1, 2 and 3 of the light chain variable region of SEQ ID NO: 40;
(v) a heavy chain variable domain comprising CDR 1, 2 and 3 of the heavy chain variable region of SEQ ID NO: 41 and (ii) a light chain variable domain comprising CDR 1, 2 and 3 of the light chain variable region of SEQ ID NO: 42;
(vi) a heavy chain variable domain comprising CDR 1, 2 and 3 of the heavy chain variable region of SEQ ID NO: 43 and (ii) a light chain variable domain comprising CDR 1, 2 and 3 of the light chain variable region of SEQ ID NO: 44; or
(vii) a heavy chain variable domain comprising CDR 1, 2 and 3 of the heavy chain variable region of SEQ ID NO: 45 and (ii) a light chain variable domain comprising CDR 1, 2 and 3 of the light chain variable region of SEQ ID NO: 46; or
(viii) a heavy chain variable domain comprising CDR 1, 2 and 3 of the heavy chain variable region of SEQ ID NO: 47 and (ii) a light chain variable domain comprising CDR 1, 2 and 3 of the light chain variable region of SEQ ID NO: 48.

45. The method of claim 44, wherein the antibody or an antigen-binding fragment thereof is covalently linked to a detectable label, cytotoxic drug, or a drug.

46. The method of claim 44, wherein the inhibitor reducing or blocking specifically the expression of an isoform of STIM1 of SEQ ID NO: 1 is selected from the group consisting of a siRNA, shRNA, antisense, ribozyme, triplex forming molecules, and aptamer specific to the mRNA encoding the isoform of STIM1 of SEQ ID NO: 1.

47. The method of claim 44, wherein the cancer is a hematopoietic cancer or a solid tumor selected from the group consisting of breast cancer, lung cancer, colon cancer, bladder cancer, leukemia, chronic lymphocytic leukemia (CLL), acute myeloid leukemia (AML), chronic myelomonocytic leukemia (CMML), endometrium cancer, melanoma, prostate cancer, pancreas cancer, glioblastoma, rectal cancer, colorectal cancer, ovary cancer, liver cancer, lung cancer, thyroid cancer, testicular cancer, myeloma (multiple myeloma), cholangiocarcinoma, nervous system cancer, uterus cancer, peritoneum cancer, digestive cancer, lymphoma and kidney cancer and the myelodysplastic syndrome is selected from the group consisting of refractory anemia, refractory anemia with ring sideroblast (RARS), refractory cytopenia with multilineage dysplasia (RCMD), refractory anemia with excess blasts, refractory anemia with excess blasts in transformation, chronic myelomonocytic leukemia, refractory cytopenia with multilineage dysplasia (RCMD), and uveal melanoma.

48. A vaccine composition comprising a peptide according to claim 32.

49. A nucleic acid encoding a heavy and/or light chain of an antibody of claim 27.
